(19) United States
(12) Patent Application Publication
(10) Pub. No.: US 2026/0347778 A1
(43) Pub. Date: Nov. 5, 2026

(54) METHODS OF PRODUCING EXTRACELLULAR VESICLES, EXTRACELLULAR VESICLES AND USES THEREOF

(71) Applicant: AUSTRALIAN NATIONAL UNIVERSITY, Canberra (AU)

(72) Inventors: Rakshanya SEKAR, Canberra (AU); Riccardo Carlo NATOLI, Canberra (AU); Yvette Sophie Maree WOOFF, Canberra (AU); Valerin Adrian CIOANCA, Canberra (AU)

(21) Appl. No.: 19/484,764

(22) PCT Filed: May 15, 2024
(86) PCT No.: PCT/AU2024/050484

§ 371 Date: Nov. 14, 2025

(30) Foreign Application Priority Data
May 15, 2023 (AU) 2023901479
May 15, 2023 (AU) 2023901480

(51) Int. Cl. C12N 5/078; A61K 35/18; A61P 25/16; A61P 27/02; A61P 29/00; C12N 15/113

(52) U.S. Cl. CPC C12N 5/0641; A61K 35/18; A61P 25/16; A61P 27/02; A61P 29/00; C12N 15/113; C12N 2310/141; C12N 2310/351; C12N 2320/32; C12N 2500/12; C12N 2500/24; C12N 2500/35; C12N 2500/46; C12N 2501/33; C12N 2501/392; C12N 2501/71

(57) ABSTRACT

The present invention provides a method of producing extracellular vesicles (EVs) comprising incubating or culturing EV producing cells in media. The present invention also provides a population of EVs and compositions comprising the vesicles and methods and uses thereof.

### FIELD OF THE INVENTION

The present invention provides a method of producing extracellular vesicles (EVs) comprising incubating or culturing EV producing cells in media. The present invention also provides a population of EVs and compositions comprising the vesicles and methods and uses thereof.

### BACKGROUND OF THE INVENTION

There is a paucity of treatments for neurodegenerative diseases. The treatments available for neurodegenerative diseases mostly treat the symptoms but not the underlying causes. Further, there are no current treatments for the majority of neurodegenerative diseases.

Neurodegeneration is a term which covers diseases or disorders involving loss of neurons. Neurodegenerative diseases may occur within the brain, or in other tissues or organs that have neurons, such as the retina. In the retina, a progressive neurological loss is typically termed retinal degenerative disease. A common form of retinal regenerative disease is age-related macular degeneration (AMD). AMD is a major cause of blindness and affects over 200 million people worldwide. Dry or atrophic AMD is a prevalent condition that affects a significant portion of the elderly population worldwide. The causes of dry AMD include family genes and environmental factors, although there is a growing recognition that oxidative stress and inflammation may play a role in developing and accelerating eye diseases such as AMD, diabetic retinopathy and retinal vein occlusion. Apart from the recently FDA approved SYFOVRE by Apellis, and IZERVAY by Astellas, there are no treatments for dry AMD, leaving patients with limited options to manage their condition. Existing treatment options for ocular diseases including AMD are limited to monthly, expensive and invasive intravitreal injections which have to be administered by ophthalmologists. Adverse immune responses have been reported with such treatments.

The treatment of neurodegenerative diseases in the brain face similar difficulties to retinal degenerative treatments. These include the immune privileged environment and blood brain barrier (BBB) (similar to the blood retinal barrier (BRB)) preventing systemic uptake of larger therapeutic molecules. Extracellular vesicles (EVs) show promise for the treatment of degenerative disease in both organs given their small nano size can penetrate the BBB and BRB, and potentially be used to deliver therapeutics to the brain and the eye. Further, unlike standard synthetic encapsulation agents which induce immune responses, naturally sourced EV can be harnessed as lowly-immunogenic delivery vehicles. EV contribution to disease onset and progression has been well established in many neurodegenerations, with cellular communication pathways altered or alterations in EV cargo.

Thus, there is a requirement for improved treatments for neurodegenerative diseases and/or eye or ocular diseases.

### SUMMARY OF THE INVENTION

The present inventors have developed methods of producing extracellular vesicles (EV) comprising incubating or culturing EV producing cells in media wherein the extracellular vesicles have advantageous properties. The present inventors have developed population of EVs and compositions comprising EVs and methods and uses thereof.

In an aspect, the present invention provides a method of producing extracellular vesicles (EVs) comprising incubating or culturing EV producing cells in media comprising an antioxidant and/or an N1 media component or an equivalent thereof.

In an aspect, the present invention provides a population of EVs derived from cells comprising an increased level one or both of an endogenous and an exogenous antioxidant.

In an aspect, the present invention provides a population of EVs produced by the method as described herein.

In an aspect, the present invention provides a population of EVs produced by the method as described herein, wherein the EVs have an increased level one or both of an endogenous and an exogenous antioxidant.

In an aspect, the present invention provides a composition comprising EVs produced by the method as described herein, or a population of EVs as described herein for use in treating and/or preventing a disease or condition in a subject.

In an aspect, the present invention provides a composition comprising EVs produced by the method as described herein, or a population of EVs as described herein for use in reducing oxidative stress and/or inflammation in a subject.

In an aspect, the present invention provides a composition comprising EVs produced by the method as described herein, or a population of EVs as described herein for use in treating and/or preventing neurodegeneration in a subject.

In an aspect, the present invention provides a method of treating and/or preventing neurodegeneration in a subject, the method comprising administering EVs produced by the method as described herein, or a population of EVs as described herein to a subject.

In an aspect, the present invention provides use of EVs produced by the method as described herein, or a population of EVs as described herein for treating and/or preventing neurodegeneration in a subject.

In an aspect, the present invention provides use of EVs produced by the method as described herein, or a population of EVs as described herein in the manufacture of a medicament for treating and/or preventing neurodegeneration in a subject.

In an aspect, the present invention provides a composition comprising EVs produced by incubating or culturing EV producing cells in media comprising an antioxidant and/or an N1 media component or an equivalent thereof for use in reducing oxidative stress and/or inflammation in the eye of a subject.

In an aspect, the present invention provides a composition comprising EVs produced by the method as described herein, or a population of EVs as described herein for use in reducing oxidative stress and/or inflammation in the eye of a subject.

In an aspect, the present invention provides a method of reducing oxidative stress and/or inflammation in the eye of a subject, the method comprising administering EVs produced by the method as described herein, or a population of EVs as described herein to a subject.

In an aspect, the present invention provides use of EVs produced by the method as described herein, or a population of EVs as described herein for reducing oxidative stress and/or inflammation in the eye of a subject.

In an aspect, the present invention provides use of EVs produced by the method as described herein, or a population of EVs as described herein in the manufacture of a medicament for reducing oxidative stress and/or inflammation in the eye of a subject.

In an aspect, the present invention provides a composition comprising EVs produced by incubating or culturing EV producing cells in media comprising an antioxidant and/or an N1 media component or an equivalent thereof for use in treating and/or preventing neurodegeneration in a subject.

In an aspect, the present invention provides a composition comprising EVs produced by incubating or culturing EV producing cells in media comprising an antioxidant and/or an N1 media component or an equivalent thereof for use in treating and/or preventing retinal degeneration in a subject.

In an aspect, the present invention provides a composition comprising EVs produced by the method as described herein, or a population of EVs as described herein for use in treating and/or preventing retinal degeneration in a subject.

In an aspect, the present invention provides a method of treating and/or preventing retinal degeneration in a subject, the method comprising administering EVs produced by the method as described herein, or a population of EVs as described herein to a subject.

In an aspect, the present invention provides use of EVs produced by the method as described herein, or a population of EVs as described herein for treating and/or preventing retinal degeneration in a subject.

In an aspect, the present invention provides use of EVs produced by the method as described herein, or a population of EVs as described herein in the manufacture of a medicament for treating and/or preventing retinal degeneration in a subject.

In an aspect, the present invention provides a method of producing EVs comprising incubating or culturing EV producing cells; or incubating or culturing EV producing cells and EV producing cell fragments in media comprising an antioxidant and/or an N1 media component or an equivalent thereof.

Any embodiment herein shall be taken to apply mutatis mutandis to any other embodiment unless specifically stated otherwise. For instance, as the skilled person would understand examples of antioxidants outlined for the methods of the invention equally apply to uses and compositions of the invention.

The present invention is not to be limited in scope by the specific embodiments described herein, which are intended for the purpose of exemplification only. Functionally-equivalent products, compositions and methods are clearly within the scope of the invention, as described herein.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

The invention is hereinafter described by way of the following non-limiting Examples and with reference to the accompanying figures.

### BRIEF DESCRIPTION OF THE ACCOMPANYING DRAWINGS

FIG. 1. Representative ERG waveform. The ERG waveform is labelled with the a-wave amplitude and implicit time and b-wave amplitude and implicit.

FIG. 2. RBC-EV isolation and characterisation. (A) Experimental paradigm for RBC-EV isolation and incubation conditions. (B) Cryo-EM imaging of RBC-EV. (C) Size distribution of RBC-EV from cryo-EM images (N=72). (D-E) Western blots for cellular marker Calnexin (CNX), EV markers Tumour susceptibility gene 101 (TSG101) and ALIX, and reference protein GAPDH. (D) CNX (90 kDa) band was present in retinal lysates, but not RBC-EV lysates. (D-E) TSG101 (50 kDa) was present in retinal, RBC and RBC-EV lysates, (E) ALIX (100 kDa) and GAPDH (37 kDa) were present in RBC and RBC-EV lysates. Scale=200 nm.

FIG. 3. RBC-EV supplementation comparison on RBC health and RBC-EV quality. (A) RBC health in incubation with or without N1 and SOD supplementation (N=2-4), with a cut-off of 10% abnormalities. (B) RBC-EV size distribution profile as measured using Nanotracking analysis (Nanosight NS300). (C) Summary data showing EV counts, % abnormality and size per mL blood in different supplementations. (D) Average EV count (N=13) (E) average mean and modal size (N-13) and (F) size distribution profile (N=3) was consistent across multiple batches RBC cultures with N1 and SOD combination supplementation.

FIG. 4. Therapeutic potential of local RBC-EV delivery with or without N1 and SOD supplementation. (A) Experimental paradigm for intravitreal injections of RBC-EV and controls. (B) Retinal function as measured by ERG shows preserved a-wave function and (C) significantly preserved b-wave function for PBS (N1/SOD) and RBC-EV (N1/SOD) compared to no supplement controls (P<0.05, N=5). (D) TUNEL+ cells in the ONL were significantly reduced in supplement groups compared to controls, while (E) photoreceptor preservation was found to be significantly increased in RBC-EV (N1/SOD) compared to PBS (N1/SOD) groups. (F) IBA-1+ cells were significantly downregulated in RBC-EV (N1/SOD) mice compared to PBS controls (P<0.05, N=5). No change was seen in the number of IBA-1+ cells in PBS (N1/SOD) or RBC-EV groups compared to PBS controls. Representative confocal images show (G) decreased TUNEL+ cells in the ONL in supplement groups as well as photoreceptor preservation in RBC-EV (N1/SOD groups) and (H) decreased IBA-1+ cells (arrows) in RBC-EV (N1/SOD) injected mice (P<0.05, N=5). Scale=50 μM.

FIG. 5. SOD and N1 and SOD combination supplementation provides protection against photo-oxidative damage in a retinal neurodegeneration model. (A) Retinal function as measured by ERG shows no change in a-wave and (B) b-wave function (P>0.05, N=5). (C) TUNEL⁺ cells in the ONL were significantly reduced in RBC-EV (SOD) and RBC-EV (N1/SOD) groups compared to RBC-EV and RBC-EV (N1)-injected mice, while (D) photoreceptor preservation was found to be significantly increased in RBC-EV (N1/SOD)-injected mice. (E) IBA-1+ cells were significantly downregulated in RBC-EV (SOD) mice (P<0.05, N=5) with no difference observed between other groups (P>0.05, N=5). (F) Whole retinal thickness was significantly increased between RBC-EV and RBC-EV (N1/SOD)-injected mice (P<0.05, N=5). Representative (G) fundus and (H) OCT images show improved retinal health in RBC-EV (SOD) and RBC-EV (N1/SOD) groups.

FIG. 6. Safety of local RBC-EV (N1/SOD) delivery. (A) Experimental paradigm. Retinal function for both (B) a-wave and (C) b-wave responses was unchanged compared to PBS-injected controls. Further, there were no significant differences in (D) TUNEL+ cells in the ONL, (E) number of photoreceptor rows, or (F) presence of IBA-1+microglia in the outer retina. (G-H) Representative confocal images show no differences in TUNEL+ cells and IBA-1+ cells in outer retina between groups. (N=5, P>0.05). Scale=50 μM.

FIG. 7. Therapeutic potential of local RBC-EV delivery. (A) Experimental paradigm for intravitreal injections of RBC-EV (N1/SOD). Retinal function as measured by ERG shows (B) significantly preserved a-wave function and (C) b-wave function compared to PBS controls. (D) TUNEL+ cells in the ONL were significantly reduced in RBC-EV (N1/SOD) compared to controls, while (E) photoreceptor preservation was found to be significantly increased in RBC-EV (N1/SOD) compared to PBS. (F) IBA-1+microglia were found to be significantly reduced in RBC-EV (N1/SOD) mice compared to PBS controls. (G-H) Representative confocal images show decreased photoreceptor cell death and presence of inflammatory cells in the outer retina in RBC-EV (N1/SOD) groups. (N=5, P<0.05). Scale=50 μM.

FIG. 8. Safety of systemic RBC-EV (N1/SOD) delivery. (A) Experimental paradigm. Retinal function for both (B) a-wave and (C) b-wave responses was unchanged compared to PBS-injected controls. Further, there were no significant differences in (D) TUNEL+ cells in the ONL, (E) the number of photoreceptor rows, or (F) presence of IBA-1+ cells in the outer retina between groups. (G-H) Representative confocal images show no differences in TUNEL+ cells and IBA-1+ cells between groups. (N=5, P>0.05). ONL=outer nuclear layer. Scale=50 μM.

FIG. 9. Therapeutic potential of systemic RBC-EV delivery. (A) Experimental paradigm for intraperitoneal injections of RBC-EV (N1/SOD). Retinal function as measured by ERG shows (B) significantly preserved a-wave function and (C) b-wave function compared to PBS controls (N=5, P<0.05). (D) TUNEL+ cells in the ONL were significantly reduced in RBC-EV (N1/SOD) compared to controls, while (E) photoreceptor preservation was found to be significantly increased in RBC-EV (N1/SOD) compared to PBS. (F) IBA-1+microglia were found to be significantly reduced in RBC-EV (N1/SOD) mice compared to PBS controls. (G) Representative confocal images show reduced TUNEL+ cells in the ONL and preserved photoreceptor thickness in RBC-EV (N1/SOD)-injected mice and (H) reduced IBA-1+ cells in the outer retina, compared to PBS. (N=15, P<0.05). ONL-outer nuclear layer. Scale=50 μM.

FIG. 10. RBC-EV (N1/SOD) uptake. (A) Experimental paradigm for in vitro and in vivo retinal cell RBC-EV (N1/SOD) uptake. (B) SYTO-labelled RBC-EV (N1/SOD) uptake in retinal cell lines 661W, BV2, MIOM1 and aRPE19 following 24 hours incubation, compared to SYTO-PBS control. (C) Transfection efficiency of SYTO-labelled RBC-EV (N1/SOD) over 24 hours, measured using the IncuCyte™ ZOOM. Technical replicates N=3. (D) SYTO-labelled RBC-EV (N1/SOD) uptake in vivo following intravitreal delivery over 7 days shows progressive radial uptake from the superior retinal delivery site, as well as penetrance through all retinal layers and across the superior and inferior retina by 7 days. (N=1) ONL=outer nuclear layer, INL=inner nuclear layer, GCL=ganglion cell layer. Scale bars=100 μm and 50 μm.

FIG. 11. Loading capacity of RBC-EV (N1/SOD). (A) Experimental paradigm for miRNA loading settings using electroporation. (B) Output measure, miRNA quantification paradigm using Qubit miRNA assay. (C-F) Electroporation optimisation settings showed that (C) miRNA retention was optimal at 350 V, 125 μF, 1 pulse (Gene Pulser), (D) with post-electroporation incubation at 37 C in BioRad Gene Pulser buffer. (E) miRNA retention did not increase with increased miRNA input loading. (F) Cryo-EM shows EV stability was maintained post-electroporation. Scale=200 nm. (N=3).

FIG. 12. N1/SOD supplementation in 661w-photoreceptor cells in vitro enhances EV production. (A) Experimental paradigm for 661w supplement incubation, in vitro photo-oxidative damage paradigm, and EV collection. (B) Nanoparticle tracking analysis (ZetaView x30) shows similar size distribution profiles for 661w-EV and 661w-EV (N1/SOD) (N=6). (C) Concentration of 661w-EV (N1/SOD) following 18 hours incubation was significantly higher in 661w-EV (N1/SOD) group compared to controls (P<0.05, N=6). (D) No significant difference was observed in mode size between groups (P>0.05, N=6). (E) 661w incubated with N1/SOD had preserved morphology and reduced cell death than controls following 2 h photo-oxidative damage. N=3.

FIG. 13. RBC antioxidant capacity is increased with N1/SOD supplementation. (A) Assay design where increased SOD activity is directly correlated to increased ROS presence. (B) RBC (N1), RBC (SOD) and RBC (N1/SOD) had significantly reduced SOD activity compared to RBC (PBS) controls (P<0.05, N=3).

FIG. 14. RBC health and quality assessments following N1/SOD incubation. RBC were assessed for morphological changes using ImageJ plug-in (A) before culture and (B) following incubation at 37 C overnight with N1/SOD supplementation. (C-F) The number of abnormalities (tear drop shape, variation in size/shape and presence of burr cells) was counted and calculated as a percentage against normal RBC.

FIG. 15. Fundal health post intravitreal injection. (A) Representative fundus images taken using MICRON IV show fundal health post intravitreal injection of PBS or RBC+/−combination supplementations and compared to PBS. (B) PBS (N1/SOD) fundal images show localised bleeds near the optic nerve in all mice.

FIG. 16. RBC-EV (N1/SOD) provides protection against sodium iodate-induced retinal degeneration. (A) Experimental paradigm. (B) Representative ERG trace for control and treated groups showing (C) observed a-wave protection in retinal function for RBC-EV (N1/SOD)-injected mice (P<0.05), but no change in (D) b-wave responses between groups (P>0.05). (E) No change was observed in photoreceptor cell death as measured by TUNEL (P>0.05); however (F) a significant reduction was measured in the total number of IBA-1⁺ immune cells in the retina (P<0.05). N=4-6.

FIG. 17. RBC-EV (N1/SOD) as a safe and efficacious therapeutic for neuroinflammatory features of Parkinson's disease. (A) Experimental paradigm. (B) Western blots against TH and IBA-1 showed (Bii) no change in TH expression within control and degenerative condition groups (P>0.05), but (Biii) a significant reduction in IBA-1 levels in 6-OHDA/RBC-EV (N1/SOD)-injected mice compared to 6-OHDA/PBS controls (P<0.05). (Ci and ii) No change was observed in rotarod or grip strength behavioural measures between any group (P>0.05). N=3-4.

FIG. 18. RBC-EV (N1/SOD) outperform commercial competitor RBC-EV products in protecting against retinal degeneration in vivo. Following 5 days photo-oxidative damage, retinal function was significantly improved for mice injected with RBC-EV (Comp) and Vehicle (V CVR) for both (A) a-wave and (B) b-wave measures (P<0.05). Retinal thickness measures on OCT images showed (C-E) significant thickness of the ONL and whole retina for RBC-EV (CVR) treated mice compared to V (CVR) and RBC-EV (Comp) groups (P<0.05). Photoreceptor survival was evidenced by (F) significantly increased numbers of photoreceptor cell rows in V (CVR) and RBC-EV (CVR) treated mice compared to other groups (P<0.05). No significant differences were observed in IBA-1⁺ cells or TUNEL⁺ cells in the outer retina, as measures of inflammation and cell death respectively (P>0.05). (N=4-5). V=vehicle, CVR=Clear Vision Research, Comp=competitor, ONL=outer nuclear layer, INL=inner nuclear layer, GCL+IPL=ganglion cell layer and inner plexiform layer, WR=whole retina.

FIG. 19. Antioxidant dose testing RBC health. Compared to pre-treatment RBC, RBC (N1/SOD) had the lowest % abnormality of all antioxidants and doses tested. RBC with Resveratrol 100 μM, Kaempferol (2 μM) and Glutathione (0.1 mM) had the lowest % abnormality out of the respective dose ranges tested (dashed lines). N=1-4.

FIG. 20. Antioxidant dose testing RBC and RBC-EV health and characterisation. (A) RBC health was assessed by the % membrane abnormalities measured, showing significantly reduced % of abnormalities in RBC (N1), RBC (SOD), RBC (N1/SOD) and RBC (R 100 μM) groups, compared to pre-treatment controls (P<0.05). (B) RBC counts were also measured following 18 h incubation with supplementations, with the highest counts seen in RBC (SOD) and RBC (N1/SOD) groups (N=1-4). Nanoparticle tracking analysis (Nanosight NS300) was performed to characterise (C) RBC-EV size distribution, (D) RBC-EV concentration and (E) RBC-EV mean and mode size, with no significant differences observed for these measures between groups. N=3.

FIG. 21. Effects of supplementations on endogenous properties of RBC and RBC-EV. (A(i)) SOD activity of RBC (N1/SOD) and RBC (K 2 μM) groups was significantly higher than RBC (PBS) controls (P<0.05), while (A(ii)) SOD activity of RBC-EV (R 100 μM) and RBC-EV (K 2 μM) was significantly higher than RBC-EV (PBS) controls (P<0.05). (B(i)) No difference was seen in haemoglobin levels between groups for RBC (P>0.05), however (B(ii)) RBC-EV (N1) and RBC-EV (R 100 μM) had significantly reduced haemoglobin levels compared to RBC-EV (PBS) controls (P<0.05). N=6.

FIG. 22. In vivo RBC-EV (Resv) did not confer protection against retinal degeneration. (A) Experimental paradigm. No changes were seen in retinal function for (B) a-wave or (C) b-wave measures between groups. (D-E) ONL was significantly thicker in RBC-EV (Resv) treated mice compared to PBS (Resv) or PBS groups (P<0.05). No differences were seen in other retinal layers (P>0.05). ONL=outer nuclear layer, INL=inner nuclear layer, GCL+IPL=ganglion cell layer and inner plexiform layer, WR=whole retina. N=5.

FIG. 23. Human RBC health. (A) Baseline abnormality score shows ~30% RBC membrane abnormality in mouse RBC and ~40% in human RBC (N=1-4). (B) Mouse RBC viability assessed by ImageStream analysis showed a significant decrease in viability as measured by the % of Calcein⁺ RBC in RBC (PBS), RBC (N1) and RBC (N1/SOD) groups compared to pre-treatment controls (P<0.05, N=3). No change was seen in the % of Annexin⁺ cells (cell death marker) between groups (P>0.05). No significant differences were observed in (C) human RBC supplementation groups for RBC counts or (D) RBC abnormalities, however RBC (SOD) and RBC (N1/SOD) groups had the lowest % abnormalities compared to pre-treatment controls (P>0.05, N=1-3). (E) RBC viability as assessed using FACS and ImageStream analyses, showing high viability as measured by Calcein⁺ cells, and low cell death as measured by Annexin V⁺ cells. No differences were detected between groups when compared to pre-treatment controls (P>0.05, N=3).

FIG. 24. Human RBC-EV health and quality was comparable between supplement groups. (A) Size distribution profile of RBC-EV using Nanoparticle tracking analysis (ZetaView x30). (B) Mean, mode, and concentration were not significantly different between groups. (N=3, p>0.05).

FIG. 25. In vitro photoreceptor-EV response to photo-oxidative damage. (A) Experimental paradigm of in vitro photo-oxidative damage to 661w photoreceptors and EV response. (B) EV distribution profile using Nanotracking analysis (ZetaView x30) shows similar size distribution profiles of 661w-EV under dim (5 k lux) and PD (25 k lux) conditions, however (C) following PD, 661w-EV were significantly increased in concentration (P<0.050 but with (D) no change in mode size (P>0.05). N=3.

FIG. 26. SOD and N1/SOD supplementation confers protection to 661w-photoreceptor cells in vitro. (A) Experimental paradigm. (B(i)-(iv)) Cell toxicity assay was performed on 661w following 18 hours incubation with supplementations (N1, 1:100; SOD, 1:500; N1/SOD, R 100 μM, K 2 μM and G 0.01 mM), before (0 hours), and after 2 hours, 3 hours, and 4 hours photo-oxidative damage (PD). (B(v)-(vi)) Comparative analyses across all time-points showed the lowest toxicity in PBS, SOD and N1/SOD treated groups compared to untreated controls. N=6.

FIG. 27. SOD and N1/SOD supplementation dose response to photo-oxidative damage in vitro. (A) Experimental paradigm. (B(i)-(iii)) Cell toxicity assay was performed on 661w following 18 hours incubation with supplementations (N1, 1:200; SOD, 1:1000; N1/SOD, R 50 μM, K 1 μM and G 0.005 mM), before (0 hours), and after 2 hours photo-oxidative damage (PD). A recovery time-point was also investigated 24 hours following 2 hours PD. (B(iv)) Comparative analyses across all time-points showed the lowest toxicity in SOD treated groups compared to untreated controls. N=6.

FIG. 28. Effects of photoreceptor-EV with supplementation to photo-oxidative damage. (A) Experimental paradigm. (B) Retinal function as measured by ERG shows no significant differences in (B) a-wave, or (C) b-wave responses between groups, (P>0.05). (D-E) Retinal thickness measurements show significant increase in ONL thickness between 661w-EV (N1/SOD) and 661w-EV groups, indicating protection following N1/SOD supplementation (P<0.05). N=5.

FIG. 29. Effects of photoreceptor-EV with supplementation to photo-oxidative damage. (A) Experimental paradigm. (B) Retinal function as measured by ERG shows no significant differences in (B) a-wave, or (C) b-wave responses between groups, (P>0.05). (D-F) Retinal thickness measurements show no significant increase in ONL thickness between 661w-EV (N1/SOD) and PBS groups or TUNEL and photoreceptor counts (P>0.05). N=5.

FIG. 30. RBC-EV have a distinct proteomic profile compared to RBC. (A) PCA of RBC vs RBC-EV. (B) Cellular compartment of RBC-EV shows terms related to extracellular vesicles. Heatmaps demonstrated enrichment of (C) EV cargo and (D) EV membrane proteins in RBC-EV compared to RBC. Pathway analyses for enriched proteins in (E (i)) RBC-EV, (E (ii)) RBC, and (E (iii)) downregulated proteins in RBC-EV compared to RBC. N=3, P<0.05.

FIG. 31. N1/SOD supplementation confers unique immune-modulatory signature to RBC. (A) Protein numbers per sample. (B) PCA showing distinct clustering between groups. (C) Heatmap of differentially expressed proteins between Pre-Tx and PBS groups. (D) Pathway analysis of differentially expressed proteins shows enrichment of hemostatic and immune pathways in RBC (PBS) groups compared to Pre-Tx controls. (E) Uniquely expressed proteins in each supplement group shows RBC (N1) samples to have the most unique proteins compared to other groups. (F) Pathway analysis of proteins within each supplement group compared to PBS and Pre-Tx groups. N=3, P<0.05.

FIG. 32. N1/SOD supplementation confers immune-modulatory properties to RBC-EV. (A) Number of proteins in each RBC-EV sample. (B) UMAP showing distinct clustering between sample groups. (C) Venn diagram showing uniquely expressed proteins within each group, highlighting the most unique proteins in RBC-EV (N1/SOD). (D) Heatmap showing a large number of downregulated proteins in RBC-EV (N1/SOD) samples compared to other groups, with € pathway analysis showing that these proteins were associated with immune processes and transcriptional regulation. N=3, P<0.05.

FIG. 33. Supplementations did not change microRNA signature of RBC-EV. (A) MicroRNA abundance in samples. (B) Top 11 microRNA abundance. (C) Pathway analysis of enriched microRNA. (D) Venn diagram showing no unique microRNA between supplement groups. N=3, P<0.05.

FIG. 34. RBC-EV promoted dose-dependent increase in PBMC viability. A dose-dependent response of increased PBMC viability was shown following RBC-EV incubation for all groups in both (A) control and (B) LPS-stimulated conditions. N=1, run in biological duplicates. Red dotted line indicates baseline (media).

FIG. 35. RBC-EV (N1/SOD) did not induce inflammatory responses in control conditions. (A-H) Cytokine output following incubation of PBMCs with RBC-EV for 48 hours. N=1, run in biological duplicates. Red dotted line indicates baseline (media). Dotted line indicates values under LLOQ. Gold dotted line indicates values above ULOQ.

FIG. 36. RBC-EV dose-dependently decrease inflammatory cytokine production in PBMC. (A-F) Cytokine output following incubation of LPS-stimulated PBMCs with RBC-EV for 48 hours. N=1, run in biological duplicates. Red dotted line indicates baseline (media). Dotted line indicates values under LLOQ. Gold dotted line indicates values above ULOQ.

### DETAILED DESCRIPTION

### General Techniques and Definitions

Unless specifically defined otherwise, all technical and scientific terms used herein shall be taken to have the same meaning as commonly understood by one of ordinary skill in the art (e.g., extracellular vesicles, proteins, neurodegenerative and/or eye diseases).

The term “and/or”, e.g., “X and/or Y” shall be understood to mean either “X and Y” or “X or Y” and shall be taken to provide explicit support for both meanings or for either meaning.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

As used herein, the term “about”, unless stated to the contrary, refers to +/−10%, more preferably +/−5%, even more preferably +/−1%, of the designated value.

As used herein, the term “subject” is any animal. In one example, the animal is a vertebrate. For example, the animal is a mammal, avian, chordate, amphibian or reptile. In an embodiment, the subject is a mammal. In a preferred embodiment, the mammal is a human. In an embodiment, the subject is a model animal, such as mouse, rat or guinea pig. In an embodiment, the subject is a mouse. In an embodiment, the mammal may be a companion animal, such as a cat, dog or horse. In an embodiment, the mammal may be livestock such as pigs, cattle, horses, goats, sheep, and deer.

As used herein, the term “increase” or “increases” or “increased” or “increasing” refers to having a higher or greater level of a given parameter compared to a control or at a baseline time point.

As used herein, the term “reduce” or “reduces” or “reduces” or “reducing” refers to having a lower level of a given parameter compared to a control or at a baseline time point.

As used herein, the terms “treat”, “treating” or “treatment” refers to at least partially obtaining a desired therapeutic outcome. In one embodiment, treating comprises preventing or delaying the appearance of one or more symptoms of a disease or condition. In one embodiment, treating comprises arresting or reducing the development of one or more symptoms of a disease or condition.

As used herein, the terms “prevent”, “preventing” or “prevention” refers to reducing the likelihood of developing a disease or condition. Prevention need not be complete and does not imply that a subject will not eventually develop the disease or condition.

As used herein, the phrase “population of extracellular vesicles” refers to any number or group or collection of extracellular vesicles as described herein. For example, a number or group of extracellular vesicles in a given solution.

As used herein, the term “endogenous” refers to originating from within, for example, produced within a cell or EV.

As used herein, the term “exogenous” refers to originating externally or outside, for example, produced externally to a cell or EV.

### Extracellular Vesicles

As used herein, the term “extracellular vesicles” or “EVs” refer to lipid bound vesicles (lipid bilayer) secreted by cells into the extracellular environment. EVs can carry cargo such as proteins, nucleic acids, lipids and metabolites. In an embodiment, the EVs of the present invention are selected from a microvesicles, exosomes and apoptotic bodies. Microvesicles, exosomes and apoptotic bodies are defined for example in Nederveen et al (2021) and Dellar et al (2022).

In an embodiment, the EVs of the present invention have a mean diameter of about 1,000 nm or less. In an embodiment, the EVs of the present invention have a mean diameter of about 800 nm or less. In an embodiment, the EVs of the present invention, have a mean diameter of about 600 nm or less. In an embodiment, the EVs of the present invention, have a mean diameter of about 500 nm or less. In an embodiment, the EVs of the present invention, have a mean diameter of about 400 nm or less.

In an embodiment, the EVs of the present invention have a mean diameter of about 20 nm to about 1000 nm, or about 20 nm to about 800 nm, or about 20 nm to about 600 nm, or about 30 nm to about 600 nm, or about 40 nm to about 400 nm, or about 30 nm to about 400 nm, or about 30 nm to about 350 nm, or about 40 nm to about 200 nm, or about 30 nm to about 200 nm. In an embodiment, the EVs have a mean diameter of about 20 nm to about 600 nm. In an embodiment, the EVs have a mean diameter of about 40 nm to about 600 nm. In an embodiment, the EVs have a mean diameter of about 40 nm to about 400 nm. In an embodiment, the EVs have a mean diameter of about 40 nm to about 200 nm. In an embodiment, the EVs have a mean diameter of about 200 nm.

In an embodiment, about 85% of the EVs have a diameter of about 200 nm of less. In an embodiment, about 80% of the EVs have a diameter of about 200 nm of less. In an embodiment, about 75% of the EVs have a diameter of about 200 nm of less.

In an embodiment, about 60% to about 70% of the EVs have a diameter of about 100 about 200 nm. In an embodiment, about 65% of the EVs have a diameter of about 100 about 200 nm. In an embodiment, about 75% to about 80% of the EVs have a diameter of about 90 to about 220 nm.

In an embodiment, the EVs of the present invention have advantageous properties for treating or preventing a disease or condition selected from the group comprising: neurodegeneration, oxidative stress and inflammation. In an embodiment, the neurodegeneration is retinal degeneration. In an embodiment, the EVs of the present invention have advantageous properties for treating oxidative stress and/or inflammation in the eye. In an embodiment, the advantageous properties include one or more of the following: favorable safety profile, biocompatible, non-immunogenic, easily manufacture on clinically relevant scales and systemic delivery. As used herein “biocompatible” refers to the EVs being autologous or being from a subject with the same blood type (O, A, B, AB) as the EV recipient.

In some embodiments, the EVs of the present invention are autologous. As used herein, “autologous” refers to being obtained from a subjects own cells (e.g. the EVs are for administration to a subject from which the EV producing cells were derived).

In some embodiments, the EVs of the present invention are heterologous. As used herein, “heterologous” refers to being obtained from a different subject of the same species (e.g. the EVs are for administration to a subject from which the EV producing cells were not derived).

In some embodiments, the EVs of the present invention are allogeneic. As used herein, “allogeneic” refers to being obtained from a donor matched subject of the same species. In an embodiment, the donor matched subject is a subject matched on one or more blood or immune markers. In an embodiment, the donor matched subject is a subject that is human leukocyte antigen (HLA) matched or partially matched.

In some embodiments, the EVs as described herein have anti-inflammatory properties. As used herein “anti-inflammatory” refers to reducing the expression of one or more cytokines, chemokines and/or inflammatory mediators in a target cell. In an embodiment, the cell is a peripheral bone mononuclear cell. In an embodiment, the cell is a target cell or in a target tissue as described herein.

In some embodiments, the EVs of the present invention reduce the expression of one or more cytokines, chemokines and/or inflammatory mediators. In an embodiment, the cytokine is selected from: MIP-1α (Ccl3), IL-1β, IL-6, IL-8, IL-10, TNFα, MCP-1 (Ccl2), and IL-1α. In some embodiments, the cytokine is MIP-1α. In some embodiments, the cytokine is IL-1β. In some embodiments, the cytokine is IL-6. In some embodiments, the cytokine is IL-8. In some embodiments, the cytokine is IL-10. In some embodiments, the cytokine is TNFα. In some embodiments, the cytokine is MCP-1. In some embodiments, the cytokine is IL-1α.

In an embodiment, the chemokine is MIP-1α or MCP-1. In an embodiment, the chemokine is MIP-1α. In an embodiment, the chemokine is MIP-1α.

In some embodiments, the EVs of the present invention reduce the expression of one or more of: MIP-1α (Ccl3), IL-6, IL-8 and MCP-1 (Ccl2).

In some embodiments, the EVs of the present invention reduce microglia activation in the subject.

In some embodiments, the EVs of the present invention modulate the expression of one or more proteins of Table 1 compared to a control. In some embodiments, the EVs of the present invention increase or decrease one or more proteins of Table 1 compared to a control.

As used herein, the term “control” refers to a reference point for quantification. The reference point may be from an internal reference (i.e. from the same source), or established data set (i.e., matched by source, cell type, EV type, supplementation type or activation status). Suitable controls will be apparent to the skilled person and/or described herein. In an embodiment, the control is a sample of EVs obtained from the EV producing cells that have not been cultured in N1, SOD or N1/SOD. In an embodiment, the control is a sample of EVs obtained from the EV producing cells that have not been cultured in N1. In an embodiment, the control is a sample of EVs obtained from the EV producing cells that have not been cultured in SOD. In an embodiment, the control is a sample of EVs obtained from the EV producing cells that have not been cultured in N1/SOD. In an embodiment, the control is a sample of EVs obtained from the EV producing cells cultured in PBS.

In some embodiments, the EVs of the present invention comprise increased expression of one or more proteins compared to a control, wherein the one or more proteins are selected from: ARIH1, RPL22, CFI, LNPK, RPS17, RPL23A, RPS21, CPOX, METAP2, FARSB, RPL18, MYG1, RPS3, RPS15A, HSP90B1, PHB1, EEF1D, DDX1, RPS4X, EIF2S3Y, AHSG, RPL12, NDUFA4, SACM1L, RPS5, RPL18A, RPL14, CALR, RPL26, RPLP0, HYOU1, ENO3, CTSE, ALDH1A7, PDIA6, PRKCSH, CLNS1A, RPS6, HACE1, RPL17, RPL27A, EPRS1, PRXL2A, TRIM56, CANX, NPEPL1, PDIA3, PPIB, EEF2, SPR, NGP, HSPA5, BAG2, SND1, RANGAP1, ENO1, RPS14, TFRC, THG1L, PFAS, PPP2R1A, GLO1 and SCAMP3.

In some embodiments, the EVs of the present invention comprise increased expression of one or more proteins compared to a control, wherein the one or more proteins are selected from: ARIH1, RPL22, CFI, LNPK, RPS17, RPL23A, RPS21, CPOX, METAP2 and FARSB. In an embodiment, the protein is ARIH1. In an embodiment, the protein is RPL22. In an embodiment, the protein is CFI. In an embodiment, the protein is LNPK. In an embodiment, the protein is RPS17. In an embodiment, the protein is RPL23A. In an embodiment, the protein is RPS21. In an embodiment, the protein is CPOX. In an embodiment, the protein is METAP2. In an embodiment, the protein is FARSB.

In some embodiments, the EVs of the present invention comprise decreased expression of one or more proteins compared to a control, wherein the one or more proteins selected from: FCHO2, CRLF3, PSMA5, GSPT1, PSMB4, ACP1, CCDC6, UROS, PSMD3, TGM2, PSMC2, PSMA3, GLRX3, RNH1, PPID, USP25, GMPR, USP5, PGLS, OSTF1, PSMB6, EIF5, TBCB, OXSR1, STIP1, USP14, PSMD5, TOLLIP, PSMD8, GPI, OTUB1, SRI, AGFG1, CFAP157, PSMD6, CHORDC1, PHPT1, PSMD7, PSMD11, PSMD12, PSMD13, GSTM5, SYNJ1, SH3GLB1, SWAP70, H4C1, PZP, PSMD14, WNK1, GDI1, PF4, SNX15, CORO1C, PTGR2, ALDOART2, IGHG3, LZIC, EPN1, PACS1, SKIC2, KYAT3, RNF213A and ANXA6.

In some embodiments, the EVs of the present invention comprise decreased expression of one or more proteins compared to a control, wherein the one or more proteins are selected from: ANXA6, RNF213, KYAT3, SKIC2, PACS1, EPN1, LZIC, IGHG3, ALDOART2 and PTGR2. In an embodiment, the protein is ANXA6. In an embodiment, the protein is RNF213. In an embodiment, the protein is KYAT3. In an embodiment, the protein is SKIC2. In an embodiment, the protein is PACS1. In an embodiment, the protein is EPN1. In an embodiment, the protein is LZIC. In an embodiment, the protein is IGHG3. In an embodiment, the protein is ALDOART2. In an embodiment, the protein is PTGR2.

In some embodiments, the EVs of the present invention comprise downregulation or upregulation of one or more pathways of Table 2 compared to a control.

In some embodiments, the EVs of the present invention comprise increased or decreased expression of one or more pathways of Table 2.

In some embodiments, the EVs of the present invention comprise increased or decreased expression of one or more proteins compared to a control, wherein the one or more proteins mediate one or more pathways of Table 2.

In some embodiments, the EVs of the present invention comprise increased or decreased expression of one or more proteins compared to a control, wherein the one or more proteins mediate one or more pathways selected from: Rab regulation of trafficking, Srp dependent cotranslational protein targeting to membrane, major pathways of rRNA processing in the nucleolus and cytosol, nonsense mediated decay independent of the exon junction complex, nonsense mediated decay nmd, raf gefs exchange gtp for gdp on rabs, cytoplasmic ribosomal proteins, trans golgi network vesicle budding, formation of a pool of free 40s subunits, Golgi associated vesicle biogenesis, MHC class II antigen presentation, Rab geranylgeranylation, signaling by ntrk1 trka, signaling by ntrks, vesicle mediated transport, eukaryotic translation initiation, signaling by receptor tyroskine kinases, neurotransmitter receptors and postsynaptic signal transmission, membrane trafficking, clathrin mediated endocytosis, Interleukin 1 family signaling, transcriptional regulation by runx1, Hedgehog ligand biogenesis, Hedgehog on state, ubiquitin mediated degradation of cdc25a, transcriptional regulation by runx3, stabilization of p53, runx1 regulates transcription of genes in differentiation of hscs, regulation of runx3 expression and activity, degradation of axin, cross presentation of soluble exogenous antigens endosomes, asymmetric localization of pcp proteins, Apc c cdh1 mediated degradation of cdc20 targeted proteins in late mitosis G1, regulation of ras by gaps, degradation of dv1, keap1 nef212 pathway, activation of apc c and apc c cdc20 mediated degradation of mitotic proteins, pcp ce pathway, transcriptional regulation by runx2, regulation of runx2 expression and activity, regulation of pten stability and activity, gli3 is processed to gli3r by the proteasome, G2 m checkpoints, auf1 hnrnp do binds and destabilizes mRNA, switching of origins to a post replicative state, Orc1 removal from chromatin, apc c mediated degradation of cell cycle proteins, Tnfr2 non canonical nf kb pathway, Ub specific processing proteases and beta catenin independent wnt signaling.

In some embodiments, the EVs of the present invention comprise increased expression of one or more proteins compared to a control, wherein the one or more proteins mediate one or more pathway/s selected from: Rab regulation of trafficking, Srp dependent cotranslational protein targeting to membrane, major pathways of rRNA processing in the nucleolus and cytosol, nonsense mediated decay independent of the exon junction complex, nonsense mediated decay nmd, raf gefs exchange gtp for gdp on rabs, cytoplasmic ribosomal proteins, trans golgi network vesicle budding, formation of a pool of free 40s subunits, Golgi associated vesicle biogenesis, MHC class II antigen presentation, Rab geranylgeranylation, signaling by ntrk1 trka, signaling by ntrks, vesicle mediated transport, eukaryotic translation initiation, signaling by receptor tyroskine kinases, neurotransmitter receptors and postsynaptic signal transmission, membrane trafficking and clathrin mediated endocytosis. In an embodiment, the EVs of the present invention comprise increased expression of one or more proteins compared to a control, wherein the one or more proteins mediate the Rab regulation of trafficking pathway. In an embodiment, the EVs of the present invention comprise increased expression of one or more proteins compared to a control, wherein the one or more proteins mediate the Srp dependent cotranslational protein targeting to membrane pathway. In an embodiment, the EVs of the present invention comprise increased expression of one or more proteins compared to a control, wherein the one or more proteins mediate the major pathways of rRNA processing in the nucleolus and cytosol pathway. In an embodiment, the EVs of the present invention comprise increased expression of one or more proteins compared to a control, wherein the one or more proteins mediate the nonsense mediated decay independent of the exon junction complex pathway. In an embodiment, the EVs of the present invention comprise increased expression of one or more proteins compared to a control, wherein the one or more proteins mediate the nonsense mediated decay nmd pathway.

In some embodiments, the EVs of the present invention comprise decreased expression of one or more proteins compared to a control, wherein the one or more proteins mediate one or more pathway/s selected from: Interleukin 1 family signaling, transcriptional regulation by runx1, Hedgehog ligand biogenesis, Hedgehog on state, ubiquitin mediated degradation of cdc25a, transcriptional regulation by runx3, stabilization of p53, runx1 regulates transcription of genes in differentiation of hscs, regulation of runx3 expression and activity, degradation of axin, cross presentation of soluble exogenous antigens endosomes, asymmetric localization of pcp proteins, Apc c cdh1 mediated degradation of cdc20 targeted proteins in late mitosis G1, regulation of ras by gaps, degradation of dv1, keap1 nef212 pathway, activation of apc c and apc c cdc20 mediated degradation of mitotic proteins, pcp ce pathway, transcriptional regulation by runx2, regulation of runx2 expression and activity, regulation of pten stability and activity, gli3 is processed to gli3r by the proteasome, G2 m checkpoints, auf1 hnrnp do binds and destabilizes mRNA, switching of origins to a post replicative state, Orc1 removal from chromatin, apc c mediated degradation of cell cycle proteins, Tnfr2 non canonical nf kb pathway, Ub specific processing proteases and beta catenin independent wnt signaling. In an embodiment, the EVs of the present invention comprise decreased expression of one or more proteins compared to a control, wherein the one or more proteins mediate the Interleukin 1 family signaling pathway. In an embodiment, the EVs of the present invention comprise decreased expression of one or more proteins compared to a control, wherein the one or more proteins mediate the transcriptional regulation by runx1 pathway. In an embodiment, the EVs of the present invention comprise decreased expression of one or more proteins compared to a control, wherein the one or more proteins mediate the Hedgehog ligand biogenesis pathway. In an embodiment, the EVs of the present invention comprise decreased expression of one or more proteins compared to a control, wherein the one or more proteins mediate Hedgehog on state pathway. In an embodiment, the EVs of the present invention comprise decreased expression of one or more proteins compared to a control, wherein the one or more proteins mediate the ubiquitin mediated degradation of cdc25a pathway. In an embodiment, the EVs of the present invention comprise decreased expression of one or more proteins compared to a control, wherein the one or more proteins mediate the transcriptional regulation by runx3 pathway. In an embodiment, the EVs of the present invention comprise decreased expression of one or more proteins compared to a control, wherein the one or more proteins mediate stabilization of p53 pathway. In an embodiment, the EVs of the present invention comprise decreased expression of one or more proteins compared to a control, wherein the one or more proteins mediate the runx1 regulates transcription of genes in differentiation of hscs pathway. In an embodiment, the EVs of the present invention comprise decreased expression of one or more proteins compared to a control, wherein the one or more proteins mediate the regulation of runx3 expression and activity pathway. In an embodiment, the EVs of the present invention comprise decreased expression of one or more proteins compared to a control, wherein the one or more proteins mediate the degradation of axin pathway. In an embodiment, the EVs of the present invention comprise decreased expression of one or more proteins compared to a control, wherein the one or more proteins mediate cross presentation of soluble exogenous antigens endosomes pathway. In an embodiment, the EVs of the present invention comprise decreased expression of one or more proteins compared to a control, wherein the one or more proteins mediate the asymmetric localization of pcp proteins pathway. In an embodiment, the EVs of the present invention comprise decreased expression of one or more proteins compared to a control, wherein the one or more proteins mediate the Apc c cdh1 mediated degradation of cdc20 targeted proteins in late mitosis G1 pathway. In an embodiment, the EVs of the present invention comprise decreased expression of one or more proteins compared to a control, wherein the one or more proteins mediate the regulation of ras by gaps pathway. In an embodiment, the EVs of the present invention comprise decreased expression of one or more proteins compared to a control, wherein the one or more proteins mediate degradation of dv1pathway. In an embodiment, the EVs of the present invention comprise decreased expression of one or more proteins compared to a control, wherein the one or more proteins mediate the keap1 nef212 pathway.

In some embodiments, the EVs of the present invention comprise exosomes. In some embodiments, the exosomes comprise a diameter of about ~40 to about 200 nm. In some embodiments, the EVs of the present invention comprise microvesicles.

In some embodiments, the microvesicles comprise a diameter of about ~40 to about 1,000 nm.

In some embodiment, the EVs of the present invention comprise apoptotic bodies.

In an embodiment, the apoptotic bodies comprise a diameter of about ~50 to about 5000 nm.

In some embodiments, the EVs are not exosomes.

In some embodiments, the EVs of the present invention can be delivered systemically (no need for ophthalmologist or anaesthesia).

In some embodiments, the EVs of the present invention can be delivered locally (e.g. intravitreally).

In some embodiments, the EVs of the present invention are suitable for treating and/or preventing ocular diseases/conditions.

In some embodiments, the EVs of the present invention are suitable for treating and/or preventing neurodegeneration.

In some embodiments, the neurodegeneration is retinal neurodegeneration.

In some embodiments, the EVs of the present invention are suitable for treating and/or preventing oxidative stress and/or inflammation in the eye of a subject.

In some embodiments, the EVs of the present invention are suitable for treating and/or preventing retinal degeneration in the eye of a subject.

In some embodiments, the protective effect (prevention/treatment) is achieved (without loading therapeutic) in both systemic and local delivery routes.

In an embodiment, the EVs as described herein have an increased level one or both of an endogenous and an exogenous antioxidant.

In an embodiment, the EVs as described herein comprises one or more N1 media components or an equivalent thereof.

In an aspect, the present invention provides a population of EVs derived from cells comprising an increased level one or both of an endogenous and an exogenous antioxidant as described herein. In an aspect, the present invention provides a population of EVs produced by the method described herein, wherein the EVs have an increased level one or both of an endogenous and an exogenous antioxidant as described herein.

In an embodiment, the SOD activity of EVs of the present invention is about 0.3 to about 0.4 U/ml for 1×10¹¹ EVs. In an embodiment, the SOD activity of the EVs is about 0.33 to about 0.37 U/ml for 1×10¹¹ EVs. EV SOD activity can be measured by any method known to a person skilled in the art, including for example the superoxide dismutase colorimetric activity kit ThermoFisher Scientific Cat. No. EIASODC.

In an embodiment, when administered systemically or locally a portion of the EVs localise to the eye. In an embodiment, when administered systemically or locally a portion of the EVs localise to the retina.

In an embodiment, when administered systemically or locally a portion of the EVs localise to the brain. In an embodiment, when administered systemically or locally a portion of the EVs localise to microglial cells. In an embodiment, when administered systemically or locally a portion of the EVs localise to glial cells. In an embodiment, when administered systemically or locally a portion of the EVs localise to an immune cell. In an embodiment, when administered systemically or locally a portion of the EVs localise to a neuron.

As used herein a “portion” is a therapeutically effective number of EVs.

In an embodiment, the EVs further comprise an exogenous cargo.

In an embodiment, the methods of producing EVs described herein increase the quality and/or quantity and/or therapeutic efficacy of the EVs produced.

As used herein, the phrase “increasing the quality of the EVs produced” includes one or more of: increasing the uniformity of the size of the EVs produced and increasing the level of one or both of an endogenous and an exogenous antioxidant in the EVs. In an embodiment, the quality is increased in comparison to EVs produced by a method described in Gangadaran et al (2018), Usman et al (2018) or Chiangjong et al (2021). In an embodiment, the quality is increased in comparison to EVs produced by calcium ionophore EV induction method (e.g. as described in Chiangjong et al, 2021).

As used herein, the phrase “uniformity of the size of the EVs produced” refers to the uniformity in the diameter of the EVs produced. The mean and modal diameter of the EVs can be assessed by methods known in the art, for example by visual assessment including microscopy, and size distribution analysis. In an embodiment, about 85% of EVs have a diameter of about 200 nm of less. In an embodiment, about 80% of EVs have a diameter of about 200 nm of less. In an embodiment, about 75% of EVs have a diameter of about 200 nm of less. In an embodiment, about 60% to about 70% of EVs have a diameter of about 100 about 200 nm. In an embodiment, about 65% of EVs have a diameter of about 100 about 200 nm. In an embodiment, about 75% to about 80% of EVs have a diameter of about 90 to about 220 nm.

As used herein, the phrase “increasing the quantity of the EVs produced” refers to increasing the total number of EVs produced for an EV-producing cell population of any given size compared to the total number of EVs produced at baseline, including in an equally sized population of EV-producing cells produced without application of the methods, and media as described herein.

As used herein, the phrase “increasing the efficacy of the EVs produced” refers to increasing the therapeutic efficacy of EVs to treat or prevent a disease or condition as described herein.

### Extracellular Vesicle Producing Cells

As used herein, the term “extracellular vesicle producing cells” or “EV producing cells” refer cells capable of producing one or more EVs when incubated or cultured in vitro. In an embodiment, the cells are derived from a subject as described herein.

In an embodiment, the cells are animal cells. In an embodiment, the cells are mammalian cells. In an embodiment, the cells are human cells. In an embodiment, the cells are rat cells.

In an embodiment, the cells are non-nucleated cells. In an embodiment, the cells are nucleated cells. In an embodiment, the cells are from a continuous cell line. In an example, the cells are from a primary cell line. In an embodiment, the cells are from an immortalized cell line.

In an embodiment, the cell line is selected from: a photoreceptor cell line, microglia cell line, muller glia cell line and retinal pigment epithelium cell line. In an embodiment, the cells are derived from a human. In an embodiment, the cells are derived from a mouse. In an embodiment, the cells are non-adherent cells (suspension cells). In an embodiment, the cells are adherent cells.

In an embodiment, the EV producing cells are selected from: red blood cells, reticulocytes, mesenchymal stem cells, epithelial cells, endothelial progenitor cells, umbilical cord cells, ocular cell line, neuronal cell line, dental pulp stem cells, dendritic cells, white blood cells, cancer cells, microglial cells, glial cells, neurons, astrocytes, photoreceptor cells, embryonic fibroblasts and megakaryocytes.

In an embodiment, the EV producing cells are red blood cells. In an embodiment, the EV producing cells are red blood cells and reticulocytes. In an embodiment, the EV producing cells are reticulocytes. In an embodiment, the EV producing cells are mesenchymal stem cells. In an embodiment, the EV producing cells are epithelial cells. In an embodiment, the EV producing cells are endothelial progenitor cells. In an embodiment, the EV producing cells are umbilical cord cells. In an embodiment, the EV producing cells are an ocular cell line. In an embodiment, the EV producing cells are a neuronal cell line. In an embodiment, the EV producing cells are dental pulp stem cells. In an embodiment, the EV producing cells are dendritic cells. In an embodiment, the EV producing cells are white blood cells. In an embodiment, the EV producing cells are cancer cells. In an embodiment, the EV producing cells are microglial cells. In an embodiment, the EV producing cells are glial cells. In an embodiment, the EV producing cells are astrocytes. In an embodiment, the EV producing cells are photoreceptor cells. In an embodiment, the EV producing cells are embryonic fibroblasts. In an embodiment, the cell is a megakaryocytes.

In an embodiment, the EV-producing cells are red blood cells, also known as RBCs or erythrocytes. RBCs can be isolated from whole blood using any method known to a person skilled in the art e.g. centrifugation or apheresis. In some embodiments, the RBCs are leukocyte depleted. Leukocyte depletion can be achieved by any method known to a person skilled in the art including for example centrifugation, washing, freezing, buffy coat removal and filtration. In an embodiment, the RBCs are passed through a leukocyte depletion filter (e.g. Sterile Acrodisc® WBC syringe filter with Leukosorb Membrane, 25 mm; Pall) to remove leukocytes.

In an embodiment, when the EV producing cells are white blood cells the white blood cells are selected from: monocytes, lymphocytes, neutrophils, eosinophils, basophils, and macrophages.

In an embodiment, the ocular cell line is a cell line as described in Lieto et al (2022), Al-Ubaidi et al (1992), Limb et al (2002) or Dunn et al (1996).

In an embodiment, the ocular cell line is selected from: a photoreceptor cell line, microglia cell line, muller glia cell line, retinal cell line and retinal pigment epithelium cell line. In an embodiment, the cells are derived from human or a mouse. In an embodiment, the ocular cell line is a retinal cell line. In an embodiment, the retinal cell line is selected from: aRPE19 endothelial cells, BV2 microglial cell line, MIO-M1 Müller cells, D407 retinal pigment epithelium cells, iMG human glia cells or primary retinal cells (photoreceptors, bipolar, microglia or recruited macrophages, Müller or RPE). In an embodiment, the retinal cell line is aRPE19. In an embodiment, the retinal cell line is BV2. In an embodiment, the retinal cell line is MIO-M1. In an embodiment, the retinal cell line is D407. In an embodiment, the retinal cell line is iMG.

In an embodiment, the EV producing cells are not stem cells. In an embodiment, the EV producing cells are not pluripotent stem cells. In an embodiment, the EV producing cells are not differentiated neural stem cells. In an embodiment, the cells are not induced pluripotent stem cells (iPSCs). In an embodiment, the EV producing cells are not mesenchymal stem cells. In an embodiment, the EV producing cells are not amniotic membrane epithelial stem cells.

In an embodiment, the EV producing stem cells are not genetically modified. For example, they are not modified to confer additional properties on the cell or EV or are not modified to comprises an exogenous cargo as described herein.

In an embodiment, the methods of producing EVs described herein increase the level of one or both of an endogenous and an exogenous antioxidant in the EV producing cells.

In an embodiment, the methods of producing EVs described herein reduce the percent of EV producing cells with abnormal morphology compared to EV producing cells not cultured in the media described herein. As used herein, the phrase “abnormal morphology” refers to abnormal morphology of the EV producing cells compared to EV producing cells not incubated or cultured in the media described herein. In an embodiment, the abnormal morphology is selected from one or more of burr cells, tear drop shape, abnormal size (larger or smaller), torn or disfigured. In an embodiment, when the cells are RBCs the morphology of the cells is compared to the morphology of RBCs in healthy blood. In an embodiment, less than about 8% of the EV producing cells have abnormal morphology. In an embodiment, less than about 10% of the EV producing cells have abnormal morphology. In an embodiment, less than about 12% of the EV producing cells have abnormal morphology.

In an embodiment, the methods of producing EVs described herein increase the percent of EV producing cells with abnormal morphology compared to the level of abnormal morphology of RBCs in healthy blood. In an embodiment, the abnormal morphology is increased about 5% compared to the level of abnormal morphology of RBCs in healthy blood. In an embodiment, the abnormal morphology is increased about 4% to about 6% compared to the level of abnormal morphology of RBCs in healthy blood. In an embodiment, the abnormal morphology is increased about 4% to about 7% compared to the level of abnormal morphology of RBCs in healthy blood.

In an aspect, the present invention provides a method of producing EVs comprising incubating or culturing EV producing cells; or incubating or culturing EV producing cells and EV producing cell fragments in media comprising an antioxidant and/or an N1 media component or an equivalent thereof. In an embodiment, the EV producing cell fragments are platelets.

“Platelets” or thrombocytes are a component of blood that plays a role in blood clotting, hemostasis and vascular functioning. In humans, platelets have no cell nucleus and are fragments of megakaryocytes. A person skilled in the art would appreciate that platelets can produce EVs.

In an embodiment, EV producing cell fragments comprise 0 to about 30% of the population of EV producing cellular material (EV producing cells and EV producing cell fragments). In an embodiment, EV producing cell fragment comprise 0 to about 25% of the population of EV producing cellular material. In an embodiment, EV producing cell fragment comprise 0 to about 20% of the population of EV producing cellular material. In an embodiment, EV producing cell fragment comprise 0 to about 15% of the population of EV producing cellular material. In an embodiment, EV producing cell fragment comprise 0 to about 10% of the population of EV producing cellular material.

In an embodiment, platelets comprise 0 to about 30% of the population of EV producing cellular material (red blood cells and platelets). In an embodiment, platelets comprise 0 to about 25% of the population of EV producing cellular material. In an embodiment, platelets comprise 0 to about 20% of the population of EV producing cellular material. In an embodiment, platelets comprise 0 to about 15% of the population of EV producing cellular material. In an embodiment, platelets comprise 0 to about 10% of the population of EV producing cellular material.

### Incubation and Culturing

In an aspect, the present invention provides a method of producing EVs comprising incubating or culturing EV producing cells in media comprising an antioxidant and/or an N1 media component or an equivalent thereof. In an aspect, the present invention provides a method of producing EVs comprising incubating or culturing EV producing cells; or incubating or culturing EV producing cells and EV producing cell fragments in media comprising an antioxidant and/or an N1 media component or an equivalent thereof. In an embodiment, the EV producing cell fragments are platelets.

As used herein, “incubating” refers to maintaining cells (or cells and cell fragments) removed from their natural environment or derived from cells removed from their natural environment viable in an artificial environment for a period of time wherein the cells are not undergoing mitosis.

As used herein, “viable” refers to working successfully outside the environment from which they were isolated and are capable of producing EVs.

As used herein, the term “culturing” or “cell culture” refers to cells (or cells and cell fragments) removed from their natural environment or derived from cells removed from their natural environment and allowed to grow (bigger and/or divide) in an artificial environment for a period of time.

Conditions of the artificial environment that may be controlled are well-known in the art and include the pH, temperature, humidity, nutrients available, atmospheric conditions, light conditions, culture substrate etc.

In an embodiment, the incubated cells are RBCs. In an embodiment, when the cells are RBCs they are diluted before addition to the media. In an embodiment, the cells are diluted with phosphate buffered saline (PBS). As used herein, “PBS” refers to PBS (phosphate buffered saline) a pH-adjusted blend of ultrapure-grade phosphate buffers and saline solutions which, when diluted to a 1× working concentration, contains, for example, 137 mM NaCl, 2.7 mM KCl, 8 mM Na₂HPO₄, and 2 mM KH₂PO₄. In an embodiment, the cells are diluted about 1:20 to about 1:5 in PBS. In an embodiment, the cells are diluted about 1:15 to about 1:7 in PBS. In an embodiment, the cells are diluted about 1:10 in PBS. In an embodiment, the cells are RBC cells and the cells are diluted about 1:10 in PBS. In an embodiment, the cells are diluted in media.

In an embodiment, the EV producing cells are incubated or cultured for about 10 hours to about 24 hours, or about 12 hours to about 20 hours, or about 12 hours to about 18 hours, or about 14 hours to about 18 hours, or about 16 hours to about 18 hours, or about 18 hours. In an embodiment, the EV producing cells are incubated or cultured for about 10 hours to about 24 hours. In an embodiment, the EV producing cells are incubated or cultured for about 12 hours to about 20 hours. In an embodiment, the EV producing cells are incubated or cultured for about 12 hours to about 18 hours. In an embodiment, the EV producing cells are incubated or cultured for about 14 hours to about 18 hours. In an embodiment, the EV producing cells are incubated or cultured for about 16 hours to about 18 hours. In an embodiment, the EV producing cells are incubated or cultured for about 18 hours. In an embodiment, the EV producing cells are incubated or cultured for about 16 hours.

In an embodiment, the EV producing cells are incubated or cultured at about 20 °C to about 42 °C, or about 22 °C to about 40 °C, or about 24 °C to about 39 °C, or about 27 °C to about 39 °C, or about 30 °C to about 39 °C, or about 33 °C to about 39 °C, or about 36 °C to about 38 °C, or about 37 °C. In an embodiment, the EV producing cells are incubated or cultured at about 20 °C to about 42 °C. In an embodiment, the EV producing cells are incubated or cultured at about 22 °C to about 40 °C. In an embodiment, the EV producing cells are incubated or cultured at about 24 °C to about 39 °C. In an embodiment, the EV producing cells are incubated or cultured at about 27 °C to about 39 °C. In an embodiment, the EV producing cells are incubated or cultured at about 30 °C to about 39 °C. In an embodiment, the EV producing cells are incubated or cultured at about 33 °C to about 39 °C. In an embodiment, the EV producing cells are incubated or cultured at about 36 °C to about 38 °C. In an embodiment, the EV producing cells are incubated or cultured at about 37 °C.

In an embodiment, the EV producing cells are not differentiated during incubation or culturing.

In addition to the EV producing cells, EV producing cell fragments may be present with the EV producing cells during the steps of the method described herein.

### Media

The EV producing cells as described herein are incubated or cultured in media comprising an antioxidant and/or an N1 media component or an equivalent thereof. As used herein, “media” refers to any gel or liquid that can support cell viability and often growth in an artificial environment. In an embodiment, the media comprises an antioxidant. In an embodiment, the media comprises at least one N1 media component. In an embodiment, the media comprises an antioxidant and N1 media.

### Antioxidants

Oxidation occurs when a substance loses electrons, for example by coming into contact with oxygen or another oxidising substance. Free radicals are produced when oxidation occurs. Free radicals disrupt normal cellular activity and participate in chain reactions that cause damage to cells and can cause severe disorders in organisms. Free radicals including hydroxyl radicals, superoxide anion radicals and hydrogen peroxide are known as reactive oxygen species (ROS).

As used herein, “antioxidants” are inhibitors of the process of oxidation. In an embodiment, the antioxidant is an enzymatic antioxidant or a non-enzymatic antioxidant.

In an embodiment, the enzymatic antioxidant is selected from one or more of: superoxide dismutase (SOD), superoxide dismutase 2 (SOD2), catalase, peroxiredoxin, glutathione peroxidase and glutathione reductase. In an embodiment, the enzymatic antioxidant is SOD. In an embodiment, the enzymatic antioxidant is SOD2. In an embodiment, the enzymatic antioxidant is catalase. In an embodiment, the enzymatic antioxidant is peroxiredoxin. In an embodiment, the enzymatic antioxidant is glutathione peroxidase. In an embodiment, the enzymatic antioxidant is glutathione reductase.

In an embodiment, the non-enzymatic antioxidant is selected from one or more of: haemoglobin, kaempferol, glutathione, vitamin E, vitamin A, vitamin C, tocopherol, carotenoid, glutathione and curcumin. In an embodiment, the non-enzymatic antioxidant is haemoglobin. In an embodiment, the non-enzymatic antioxidant is kaempferol. In an embodiment, the non-enzymatic antioxidant is glutathione. In an embodiment, the non-enzymatic antioxidant is vitamin E. In an embodiment, the non-enzymatic antioxidant is vitamin A. In an embodiment, the non-enzymatic antioxidant is vitamin C. In an embodiment, the non-enzymatic antioxidant is tocopherol. In an embodiment, the non-enzymatic antioxidant is carotenoid. In an embodiment, the non-enzymatic antioxidant is glutathione. In an embodiment, the non-enzymatic antioxidant is curcumin.

In an embodiment, the antioxidant is endogenous antioxidant. As used herein, the “endogenous antioxidant” is an antioxidant is produced within the host from which the EV producing cell is obtained or is produced within the EV producing cell. In an embodiment, the endogenous antioxidant is an enzymatic antioxidant. In an embodiment, the endogenous antioxidant is a non-enzymatic antioxidant.

In an embodiment, the endogenous antioxidant is selected from one or more of: haemoglobin, SOD, glutathione, vitamin C, vitamin E, catalase and glutathione peroxidase. In an embodiment, the endogenous antioxidant is haemoglobin. In an embodiment, the endogenous antioxidant is SOD. In an embodiment, the endogenous antioxidant is glutathione. In an embodiment, the endogenous antioxidant is vitamin C. In an embodiment, the endogenous antioxidant is vitamin E. In an embodiment, the endogenous antioxidant is catalase. In an embodiment, the endogenous antioxidant is glutathione peroxidase.

In an embodiment, the antioxidant is an exogenous antioxidant. For example, exogenous antioxidants are provided to the cell or organism by an external source, such as through feeding or supplementation during incubation or culturing. In an embodiment, the exogenous antioxidant is selected from SOD and kaempferol. In an embodiment, the exogenous antioxidant is SOD. In one embodiment, an antioxidant comprises one or both of an enzymatic antioxidant and a non-enzymatic antioxidant, examples of such antioxidants are described herein.

### N1 Media Component or Equivalent Thereof

As used herein, “N1 media” also referred to as “N1 medium supplement” is a commercially available media comprising 0.5 mg/ml human insulin, 0.5 mg/ml human transferrin (partially iron-saturated), 0.5 μg/ml sodium selenite, 1.6 mg/ml putrescine, and 0.73 μg/ml progesterone. The media is prepared in Earle's Balanced Salt Solution (EBSS) without phenol red.

As used herein, “N1 media component” refers to one or more of transferrin, insulin, sodium selenite, putrescine and progesterone.

As used herein, an “equivalent thereof” refers to a functional equivalent of an N1 media component.

It will be apparent to those skilled in the art that N1 media components may be altered (derivative) or swapped for equivalents thereof. In an embodiment, the equivalent has at least 80%, or at least 85%, or at least 90%, or at least 95%, or at least 98% function of a selected N1 media component. In an embodiment, the equivalent has at least 80% function of the selected N1 media component. In an embodiment, the equivalent has at least 85% function of the selected N1 media component. In an embodiment, the equivalent has at least 90% function of the selected N1 media component. In an embodiment, the equivalent has at least 95% function of the selected N1 media component. In an embodiment, the equivalent has at least 98% function of the selected N1 media component.

In an embodiment, an equivalent of human insulin is an insulin derived from another organism e.g. a bovine or porcine insulin. In an embodiment, the equivalent of human insulin is a derivative of human insulin (a synthetic insulin) or a derivative of an insulin derived from another organism.

In an embodiment, an equivalent of transferrin is transferrin derived from another organism e.g. bovine or porcine transferrin. In an embodiment, an equivalent of transferrin is another protein that binds to and transports iron in the blood e.g. lactoferrin or ceruloplasmin.

Selenium is an essential trace element that can be found in different forms e.g. as selenite, selenate and selenomethionine. Sodium selenite is the most common form used for supplementation. In an embodiment, the equivalent of sodium selenite is selenium/selenite in another form e.g. selenite, selenate and selenomethionine.

In an embodiment, an equivalent of progesterone is progesterone derived for another organism e,g. bovine or porcine. In an embodiment, an equivalent of progesterone is a progesterone derivative.

In an embodiment, the media comprises at least two N1 media components. In an embodiment, the media comprises at least three N1 media components. In an embodiment, the media comprises at least four N1 media components. In an embodiment, the media comprises five N1 media components. In an embodiment, the media comprises an N1 media component equivalent. In an embodiment, the media comprises at least two N1 media component equivalents. In an embodiment, the media comprises at least three N1 media component equivalents. In an embodiment, the media comprises four N1 media component equivalents. In an embodiment, the media comprises an antioxidant and at least one N1 media component. In an embodiment, the media comprises an antioxidant and at least two N1 media components. In an embodiment, the media comprises an antioxidant and at least three N1 media components. In an embodiment, the media comprises an antioxidant and at least four N1 media components. In an embodiment, the media comprises an antioxidant and five N1 media components.

In an embodiment, the media comprises an antioxidant and at least one N1 media component equivalent. In an embodiment, the media comprises an antioxidant and at least two N1 media component equivalents. In an embodiment, the media comprises an antioxidant and at least three N1 media component equivalents. In an embodiment, the media comprises an antioxidant and four N1 media component equivalents.

In an embodiment, the media comprises all N1 media components (comprises N1 media). In an embodiment, the media comprises N1 media and SOD.

In an embodiment, the media comprises N1 media at a concentration of about 1 μg/ml to 10 μg/ml. In an embodiment, the media comprises N1 media at a concentration of about 1 μg/ml to 5 μg/ml. In an embodiment, the media comprises N1 media at a concentration of about 5 μg/ml to 10 μg/ml. In an embodiment, the media comprises N1 media at a concentration of about 3 μg/ml to 7 μg/ml. In an embodiment, the media comprises N1 media at a concentration of about 4 μg/ml to 6 μg/ml. In an embodiment, the media comprises N1 media at a concentration of about 1 μg/ml. In an embodiment, the media comprises N1 media at a concentration of about 2 g/ml. In an embodiment, the media comprises N1 media at a concentration of about 3 μg/ml. In an embodiment, the media comprises N1 media at a concentration of about 4 g/ml. In an embodiment, the media comprises N1 media at a concentration of about 5 g/ml. In an embodiment, the media comprises N1 media at a concentration of about 6 μg/ml. In an embodiment, the media comprises N1 media at a concentration of about 7 g/ml. In an embodiment, the media comprises N1 media at a concentration of about 8 μg/ml. In an embodiment, the media comprises N1 media at a concentration of about 9 μg/ml. In an embodiment, the media comprises N1 media at a concentration of about 10 μg/ml.

In an embodiment, the media comprises SOD at a concentration of about 1 μg/ml to 10 μg/ml. In an embodiment, the media comprises SOD at a concentration of about 1 μg/ml to 5 μg/ml. In an embodiment, the media comprises SOD at a concentration of about 5 μg/ml to 10 μg/ml. In an embodiment, the media comprises SOD at a concentration of about 3 μg/ml to 7 μg/ml. In an embodiment, the media comprises SOD at a concentration of about 4 μg/ml to 6 μg/ml. In an embodiment, the media comprises SOD at a concentration of about 1 μg/ml. In an embodiment, the media comprises SOD at a concentration of about 2 μg/ml. In an embodiment, the media comprises SOD at a concentration of about 3 μg/ml. In an embodiment, the media comprises SOD at a concentration of about 4 μg/ml. In an embodiment, the media comprises SOD at a concentration of about 5 μg/ml. In an embodiment, the media comprises SOD at a concentration of about 6 μg/ml. In an embodiment, the media comprises SOD at a concentration of about 7 μg/ml. In an embodiment, the media comprises SOD at a concentration of about 8 μg/ml. In an embodiment, the media comprises SOD at a concentration of about 9 μg/ml. In an embodiment, the media comprises SOD at a concentration of about 10 μg/ml.

In an embodiment, the media comprises N1 media and SOD at a concentration of about 1 μg/ml. In an embodiment, the media comprises N1 media and SOD at a concentration of about 2 μg/ml. In an embodiment, the media comprises N1 media and SOD at a concentration of about 3 μg/ml. In an embodiment, the media comprises N1 media and SOD at a concentration of about 4 μg/ml. In an embodiment, the media comprises N1 media and SOD at a concentration of about 5 μg/ml. In an embodiment, the media comprises N1 media and SOD at a concentration of about 6 μg/ml. In an embodiment, the media comprises N1 media and SOD at a concentration of about 7 μg/ml. In an embodiment, the media comprises N1 media and SOD at a concentration of about 8 μg/ml. In an embodiment, the media comprises N1 media and SOD at a concentration of about 9 μg/ml. In an embodiment, the media comprises N1 media and SOD at a concentration of about 10 μg/ml.

### Additional Media/Supplement

The media as described herein comprising an antioxidant and/or an N1 media component or an equivalent thereof may comprise one or more of an additional media and a supplement. Such additional media will be known to the skilled person (see, for example, Josefsberg et al., 2012; Wolf et al., 2011) and include any media in which an EV producing cell can be incubated or cultured that does not prevent the production of EVs. Additional media for incubation or culturing EV producing cells of the present invention include, but are not limited to: Iscove's medium, Opti-MEM UltraCHO, CD Hybridoma serum free medium, episerf medium, MediV SF103 (serum free medium), a stem cell medium or supplement (e.g. from Stem cell technologies), Dulbecco's modified eagle medium (DMEM), Eagles Modified Eagle Medium (EMEM), Glasgow's modified eagle medium (GMEM), SMIP-8, modified eagle medium (MEM), VP-SFM, DMEM based SFM, DMEM/F12, DMEM/Ham's F12, VPSFM/William's medium E, ExCell 525 (SFM), adenovirus expression medium (AEM) and Excell 65629. In an embodiment, the additional medium is not DMEM/F12. An additional supplement may include for example a calcium ionophore (to aid in the stimulation of EV production.

It will be appreciated by persons skilled in the art that the media comprising an antioxidant and/or an N1 media component or an equivalent thereof may be supplemented with additional growth factors, for example, but not limited, amino acids, vitamins, carbohydrates, inorganic salts, glucose, basic and trace elements, serum, growth factors, attachment factors, hormones, buffering systems, supplements, antibiotics and minerals. In some embodiments, the media is not supplemented with serum. In some embodiments, the media does not comprise a component produced in a human. In some embodiments, the media does not comprise a component produced in an animal.

In some embodiments, the media does not comprise one or more of: differentiation factors, neural differentiation factors, neurotrophic factors and growth factors.

In some embodiments, the media does not comprise one or more of the following: corticosterone, a calcium ionophore, triiodothyronine, levocarnitine, linoleic acid, alpha-lipoic acid, levocarnitine HCL, vitamin H, dorsomorphin, alkaline fibroblast growth factor, biotin and pancreas islet element. In an embodiment, the medium does not comprise corticosterone. In an embodiment, the medium does not comprise a calcium ionophore. In an embodiment, the medium does not comprise triiodothyronine. In an embodiment, the medium does not comprise levocarnitine. In an embodiment, the medium does not comprise linoleic acid. In an embodiment, the medium does not comprise alpha-lipoic acid. In an embodiment, the medium does not comprise levocarnitine HCL. In an embodiment, the medium does not comprise vitamin H. In an embodiment, the medium does not comprise dorsomorphin. In an embodiment, the medium does not comprise alkaline fibroblast growth factor. In an embodiment, the medium does not comprise biotin. In an embodiment, the medium does not comprise pancreas islet element.

In an embodiment, the cells are not cultured or incubated under hypoxic conditions.

### Extracellular Vesicle Isolation

In an embodiment, the EVs are isolated or separated from the cells after incubation or culturing. EV isolation or separation may be performed by any method known to a person skilled in the art including e.g. centrifugation, filtration, tangential flow filtration, size exclusion chromatography, nano-flow/facs, magnetic beads, EV isolation kits (e.g. ExoQuick® ULTRA EV Isolation System, system biosciences cat no. EQULTRA-20A-1), extrusion. In an embodiment, EVs are isolated from RBCs after incubation. In an embodiment, isolation requires centrifugation and removal of the supernatant (EVs are present in the precipitate).

### Endogenous and Exogenous Cargo

In an embodiment, the EVs of the present invention comprise an endogenous or an exogenous cargo. In an embodiment, the EVs of the present invention comprise an endogenous cargo (e.g. an endogenous antioxidant, DNA or RNA molecule). The cargo may be within the vesicle, within the lipid membrane of the vesicle and/or on the surface of the vesicle. In an embodiment, the cargo is within the vesicle. In an embodiment, the cargo is within the lipid membrane of the vesicle. In an embodiment, the cargo is on the surface of a vesicle.

In some embodiments, the endogenous cargo is an antioxidant. In an embodiment, the antioxidant is selected from one or more of: haemoglobin, kaempferol, glutathione, vitamin E, vitamin A, vitamin C, tocopherol, carotenoid, glutathione and curcumin. In an embodiment, the antioxidant is haemoglobin.

In some embodiments, the endogenous cargo is miRNA. In an embodiment, the miRNA is selected from one or more of: mmu-miR-142a-3p, mmu-miR-486b-3p, mmu-let-7c-5p, mmu-miR-16-5p, mmu-miR-25-3p, mmu-miR-486a-3p, mmu-miR-486b-5p, mmu-miR-486a-5p, mmu-let-7f-5p, mmu-let-7a-5p and mmu-miR-451a. In an embodiment, the miRNA is mmu-miR-142a-3p. In an embodiment, the miRNA is mmu-miR-486b-3p. In an embodiment, the miRNA is mmu-let-7c-5p. In an embodiment, the miRNA is mmu-miR-16-5p. In an embodiment, the miRNA is mmu-miR-25-3p. In an embodiment, the miRNA is mmu-miR-486a-3p. In an embodiment, the miRNA is mmu-miR-486b-5p. In an embodiment, the miRNA is mmu-miR-486a-5p. In an embodiment, the miRNA is mmu-let-7f-5p. In an embodiment, the miRNA is mmu-let-7a-5p. In an embodiment, the miRNA is mmu-miR-451a.

In some embodiments, the EVs of the present invention comprises an exogenous cargo. In an embodiment, the cargo is a therapeutic cargo. In an embodiment, the exogenous therapeutic cargo is selected from one or more of a: drug, antioxidant, chemotherapy, protein, lipid, nucleic acid (such as DNA, mRNA, miRNAs, siRNAs, circular RNA, long non-coding RNA and snoRNAs), CRISPR/Cas9, nanoparticles and an exogenous targeting molecule. In an embodiment, the exogenous cargo is a drug. In an embodiment, the drug is a retinal treatment drug or a drug for reducing inflammation and/or oxidative stress in the eye. In an embodiment, the drug is selected from: corticosteroid, a drug that targets the inflammasome, a drug that targets a complement protein, an anti-VEGF antibody (e.g. Lucentis or Bevacizumab), and Syfovre. In an embodiment, the exogenous cargo is an antioxidant. In an embodiment, the exogenous cargo is a chemotherapeutic. In an embodiment, the exogenous cargo is a protein. In an embodiment, the exogenous cargo is a lipid. In an embodiment, the exogenous cargo is a nucleic acid. In an embodiment, the exogenous cargo is a DNA. In an embodiment, the exogenous cargo is a mRNA. In an embodiment, the exogenous cargo is a miRNA. In an embodiment, the exogenous cargo is a siRNA. In an embodiment, the exogenous cargo is a circular RNA. In an embodiment, the exogenous cargo is a long non-coding RNA. In an embodiment, the exogenous cargo is a snoRNA. In an embodiment, the exogenous cargo is a CRISPR/Cas9. In an embodiment, the exogenous cargo is a nanoparticle. In an embodiment, the cargo is an exogenous targeting molecule.

In some embodiments, the EVs as described herein do not comprise exogenous cargo.

As used herein, an “exogenous targeting molecule” is a molecule that facilitates targeting the EV to a specific site within a subject after administration. In an embodiment, the exogenous targeting molecule facilitates targeting to an ocular cell or tissue. In an embodiment, the exogenous targeting molecule facilitates targeting to a neural cell or tissue. In an embodiment, the exogenous targeting molecule facilitates targeting to the retina. In an embodiment, the exogenous targeting molecule facilitates targeting to the neural cell or tissue susceptible to neurodegeneration. In an embodiment, the exogenous targeting molecule facilitates targeting to the ocular cell or tissue susceptible to neurodegeneration.

In some embodiments, the EVs as described herein do not comprise an exogenous targeting molecule. In some embodiments, the EVs as described herein do not comprise the exogenous targeting molecule PDGFA.

In an embodiment, exogenous cargo is loaded into an EV by the EV producing cell (the EV producing cell produces an EV that comprises (has packaged) at least some of the cargo present within the cell). In such embodiments, the exogenous cargo may be provided to the EV producing cell in the media or introduced in the EV producing cell via e.g. electroporation, sonication, incubation, transfection or mechanical penetration.

In an alternate embodiment, chemical or biomolecules can be directly loaded into isolated EVs using techniques known in the art e.g. electroporation, sonication, incubation, transfection or mechanical penetration.

### Diseases and Conditions

Exemplary diseases and conditions to be treated and/or prevent using the EVs, compositions, methods and uses as described herein include disease and conditions which cause and/or results in oxidative stress and/or inflammation. In an embodiment, the disease or condition is a neurodegenerative disease or condition. In an embodiment, the disease or condition is oxidative stress and/or inflammation in the eye. In an embodiment, the disease or condition is retinal degeneration.

In an embodiment, the disease or condition is associated with elevated levels of one or more chemokines, cytokines and/or inflammatory mediators.

As used herein “oxidative stress” refers to an increase/accumulation of reactive oxygen species in cells, tissues or organs. When there is an imbalance of reactive oxygen species, also known as free radicals, and the body's innate antioxidant defences, this results in oxidative stress in the body. The presence of oxidative stress and/or reactive oxygen species can be measured by any method known to a person skilled in the art e.g. indirectly by measuring DNA/RNA damage, lipid peroxidation and protein oxidation/nitration.

As used herein “inflammation” refers to both the normal bodily function of induction of inflammatory molecules in response to injuries and pathogens or other substances, as well as an imbalance or excess in inflammatory molecules that can result in a range of chronic diseases.

As used herein “oxidative stress in the eye” refers to an increase/accumulation of reactive oxygen species in the eye.

As used herein “inflammation in the eye” refers to an increase in inflammatory processes and inflammatory molecules in the eye. Symptoms can include eye redness, eye pain, light sensitivity, blurred vision and decreased vision.

In an embodiment, the neurodegenerative disease or condition that causes and/or is the result of oxidative stress and/or inflammation.

In an embodiment, the neurodegenerative disease or condition that causes and/or is the result of microglia activation and/or proliferation.

In an embodiment, the neurodegenerative disease or condition comprises neuroinflammation.

In an embodiment, the neurodegenerative disease or condition is retinal degeneration.

In an embodiment, the disease or condition that causes and/or is the result of oxidative stress and/or inflammation in the eye is selected from: macular degeneration, retinitis pigmentosa, diabetic retinopathy, stargardt disease, leber congenital amaurosis, best disease, cone-rod dystrophy, usher syndrome, choroideremia, bardet-biedl syndrome, refsum disease, macula telangana, macular telangiectasia, macular oedema, retinal detachment, retinal ischemia, uveitis, scleritis, conjunctivitis, keratitis, corneal ulcer, glaucoma trachoma, choroidal melanoma, ocular melanoma, glaucoma retinal dystrophy, strabismus and cataracts.

In an embodiment, the disease or condition that causes and/or is the result of oxidative stress and/or inflammation in the eye is selected from: macular degeneration, retinitis pigmentosa, diabetic retinopathy, stargardt disease, macular oedema retinal detachment and retinal ischemia.

In an embodiment, the disease or condition that causes and/or is the result of oxidative stress and/or inflammation in the eye is selected from: retinal degeneration, glaucoma, diabetic retinopathy and retinal vein occlusion.

In an embodiment, the disease or condition that causes and/or is the result of oxidative stress and/or inflammation in the eye is retinal degeneration.

In an embodiment, the retinal degeneration is selected from: macular degeneration, retinitis pigmentosa, diabetic retinopathy, stargardt disease, leber congenital amaurosis, best disease, cone-rod dystrophy, usher syndrome, choroideremia, bardet-biedl syndrome, refsum disease, macula telangana, macular odema, retinal detachments and retinal ischemia. In an embodiment, the macular degeneration is selected from wet macular degeneration and dry macular degeneration. In an embodiment, the macular degeneration is geographic atrophy. Geographic atrophy refers to the later-stages of dry AMD.

In an embodiment, the neurodegenerative disease or condition is Parkinson's disease (PD). PD is the second most common age-associated neurodegenerative disorder. Chronic neuroinflammation is a key hallmark of the pathophysiology of PD (Araújo et al., 2022). Infiltration and accumulation of immune cells from the periphery are detected in and around the affected brain regions of PD patients.

In an embodiment, the neurodegenerative disease or condition is Amyotrophic Lateral Sclerosis (ALS) (also known as Motor Neurone disease/Lou Gehrigs). ALS is characterised by neuroinflammation. Neuroinflammation in ALS comprises infiltration of lymphocytes and macrophages, activation of microglia and reactive astrocytes, as well as the involvement of complement (Rojas et al., 2020; Mead et al., 2023; Benatar et al., 2022).

In an embodiment, the neurodegenerative disease or condition is Alzheimer's disease. Patients with AD have increased levels of inflammatory markers (Leng et al., 2021; Sánchez-Sarasúa et al., 2020).

In an embodiment, the neurodegenerative disease or condition is a taupathy. A person skilled in the art will appreciate that tauopathies are a heterogeneous group of neurodegenerative disorders characterised by aggregation of protein tau into filamentous inclusions within neurons and glia. Tau pathology can independently arise secondary to a range of triggers that are each associated with inflammatory processes including, for example, infection, repetitive mild traumatic brain injury, seizure activity, and autoimmune disease (Cherry et al., 2022; Laurent et al., 2018).

In an embodiment, the neurodegenerative disease or condition is Multiple Sclerosis (MS). MS is a complex disease comprising one or more of neuroinflammation, demyelination and sequential axonal loss. MS is the most common inflammatory, demyelinating, neurodegenerative disorder of the central nervous system (Naegele et al., 2014; Bjelobaba et al., 2017).

In an embodiment, the neurodegenerative disease or condition is Lewy Body Dementia (also known as Dementia with Lewy Bodies or DBL). DBL is the second most common neurodegenerative cause of dementia (Amin et al., 2020).

In an embodiment, the neurodegenerative disease or condition is stroke or transient ischemic attack. Stroke is the third leading cause of death and disability worldwide. Neuroinflammation is a major pathological event involved in the process of ischemic injury and repair (Jayaraj et al., 2019). In particular, microglia play a dual role in neuroinflammation.

In an embodiment, the neurodegenerative disease or condition is Huntington's disease (HD). HD is a devastating neurodegenerative genetic disorder that causes progressive motor dysfunction, emotional disturbances, and cognitive impairment. Neuroinflammation is a component of HD, and modulation of neuroinflammation has been suggested as a potential target for therapeutic intervention. Neuroinflammation in the HD brain comprises reactive morphology in these glial cells and a chronic inflammatory state (Palpagama et al., 2019; Lee et al., 2021).

In an embodiment, the condition is not a demyelinating disease or condition.

In some embodiments, the EVs of the present invention are administered during or after an ocular surgical procedure to aid post-surgical recovery where inflammation and/or oxidative stress is present (e.g. retinal detachment, cataract surgery).

In some embodiments, the EVs of the present invention are administered during or after a surgical procedure to aid post-surgical recovery where neuroinflammation and/or oxidative stress is present.

In some embodiments, the EVs of the present invention reduce the expression of one or more cytokines, chemokines and/or inflammatory mediators in a disease or condition as described herein.

In some embodiments, the one or more cytokines, chemokines and/or inflammatory mediators are selected from the group comprising: MIP-1α (Ccl3), IL-1β, IL-6, IL-8, IL-10, TNFα, MCP-1 (Ccl2), and IL-1α. In an embodiment, the cytokine is MIP-1α. In an embodiment, the cytokine is IL-1β. In an embodiment, the cytokine is IL-6. In an embodiment, the chemokine is IL-8. In an embodiment, the cytokine is IL-10. In an embodiment, the cytokine is TNFα. In an embodiment, the chemokine is MCP-1. In an embodiment, the cytokine is IL-1α.

### Administration

The EVs or compositions as described herein can be administered to a subject by an appropriate route, either alone or in combination with a therapeutic agent.

A variety of routes of administration are possible including, but not limited to, systemic and local routes of administration. In an embodiment, the delivery route is selected from: intravenous, intra-arterial, intramuscular, intradermal, intravascular, oral or subcutaneous injection routes. In an embodiment, the systemic delivery is intravenous delivery. In an embodiment, the systemic delivery is subcutaneous injection.

In an embodiment, the delivery route is selected from: topical administration to the eye (e.g. eye drops), intraocular injection, subretinal injection, periocular injection, intravitreal injection, subconjunctival injection, intracranial injection, intrathecal injection, intracerebral infusion and intracerebral implantation. In an embodiment, the local delivery is topical administration. In an embodiment, the local delivery is intravitreal injection. In an embodiment, the local delivery is subretinal injection.

In an embodiment, the EVs or compositions as describe herein are administered systemically or locally and localise to an ocular cell or tissue. In an embodiment, the EVs or compositions as describe herein are administered systemically or locally and localise to a neural cell or tissue. In an embodiment, the neural cell is selected from a neuron, glia, microglia and astrocytes.

In one embodiment, the EVs or compositions as described herein are administered systemically or locally and localise to the retina. In an embodiment, the retinal cell is selected from one or more of: photoreceptor, ganglion cell, bipolar cell, muller cell, microglia/macrophages, retinal pigment epithelial cell and endothelial cell. In one embodiment, the EVs or compositions as described herein are administered systemically or locally and localise to a retinal blood vessel.

Formulation of the composition to be administered will vary according to the route of administration selected (e.g., systemic or local). In an embodiment, the formulation comprises PBS.

A composition comprising the EVs as described herein may contain a physiologically acceptable carrier. For solutions or emulsions, suitable carriers include aqueous or alcoholic/aqueous solutions, emulsions or suspensions, including saline and buffered media. Parenteral carriers include sodium chloride solution, Ringer's dextrose, dextrose and sodium chloride, lactated Ringer's or fixed oils. Intravenous carriers include various additives, preservatives, or fluid, nutrient or electrolyte replenishers and the like.

### Examples

### Example 1: Materials and Methods

### Animal Paradigms

### Animal Handling

Adult male and female C57BL/6J wild-type (WT) mice (aged 60-80 postnatal days; (P60-80) at experimental onset) were purchased from the Animal Resources Centre (ARC), (Canning Vale, Western Australia (WA)). Mice were bred, reared and housed under 12 hour light/dark cycle conditions (5 lux) with free access to food and water.

### Photo-Oxidative Damage

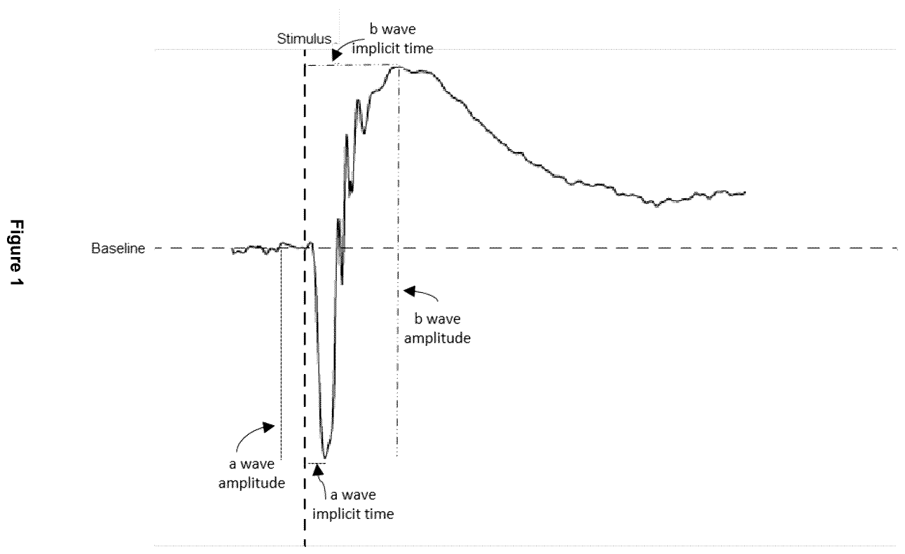

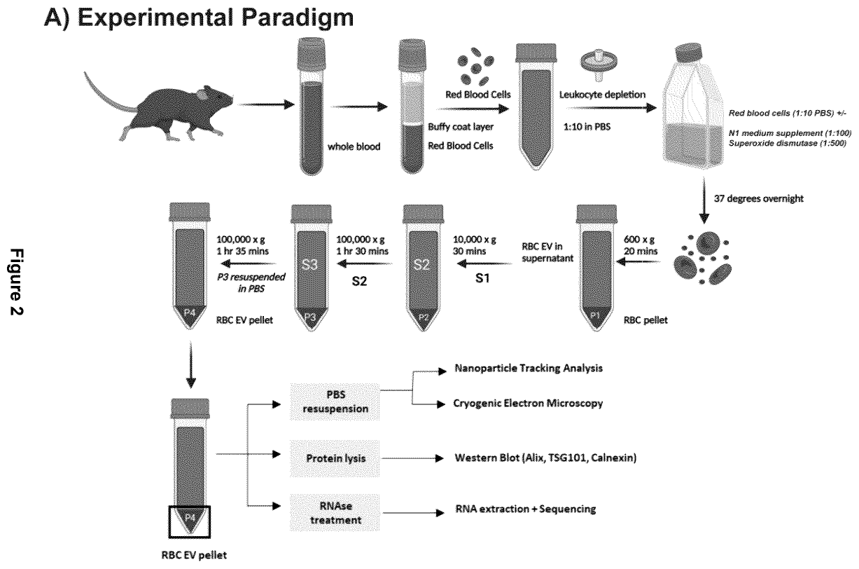

Mice were subjected to photo-oxidative damage (PD) for 5 days as described previously in Natoli et al (2016). Briefly, mice were placed into Perspex boxes coated with a reflective interior surface and exposed to 100 K lux white light from light-emitting diodes (LED). Animals were administered pupil dilator (Minims® atropine sulphate 1% w/v; Bausch and Lomb) to both eyes twice a day (9 am and 4 μm) during the course of the damage paradigm. Mice were euthanised with CO₂ following experimental runs. A representative EGR waveform is shown in FIG. 1. A-wave and b-wave amplitudes and implicit times were plotted and analysed in GraphPad Prism 9 and the data expressed as the mean wave amplitude or implicit time±SEM (Standard Error of the Mean) (μV).

Induction of Retinal Degeneration with Sodium Iodate

To induce chemical retinal degeneration, 50 mg/kg sodium iodate in H₂O was injected into the intraperitoneal cavity of C57BL6/J mice (8-12 weeks old). This dosage was selected as to not induce toxicity, but still cause significant damage to the eye as described in Koster et al (2022). Sodium iodate injections were conducted on the morning of day 0. Mice were then treated in accordance with the treatment regimen as described below. On the evening of the final day of treatment, mice were placed into dark adaptation overnight for functional and morphological assessments the following morning. Animals were checked daily throughout this paradigm for signs of discomfort and to ensure no ill-effects of any compounds injected.

### Induction of 6-OHDA Lesion

Mice received bilateral intra-cerebral injections into the striatum, with one side serving as a control and the other side experimental as per Masini et al (2021). The experimental side received either a vehicle injection or was lesioned with 6-OHDA; this was followed by a treatment injection of therapeutic RBC-EV, or vehicle. The injections were conducted at stereotaxic coordinates Anterior/Posterior +0.6 mm; Medial/Lateral±2.2 mm; Dorsal/Ventral −3.2, relative to the bregma.

The mouse was mounted in the stereotaxic frame and once stability of anaesthesia was achieved, the top of the head was shaved, and a midline incision was made to expose the skull. The injection site was marked on the skull (Anterior/Posterior, medial/lateral coordinates), and a small pilot hole was drilled to allow for insertion of the needle.

A borosilicate pipette (50 μm tip) was inserted into the brain through the hole, guided by the stereotaxic frame, and mice were injected with 1 μL of 6-OHDA hydrochloride (4 microgram/microlitres) in 0.9% sterile saline and 0.02 mg/mL % of ascorbic acid, or an equivalent volume of sterile saline and ascorbic acid. In the experimental side, mice then received 1 μL of RBC-EV (N1/SOD), or equivalent volume of PBS.

To reduce the high mortality rate historically observed during this procedure, an “enhanced care” regime was utilized. This regime involves timely interventions to address the animals' needs as per Masini et al (2021). Notably, mice were provided with water-softened chow pellets, and daily subcutaneous meloxicam injections (5 mg/kg) for the 72 hours following surgery.

This model of Parkinson's disease has previously been shown in Masini et al (2021) to induce early molecular changes associated with neuroinflammation as early as 3-7 days post-induction, with major behavioural deficits by 3-4 weeks. For this reason, and to focus on early treatment potential, a 7-day time-point was chosen to assess molecular and behavioural responses to treatment.

### Rotarod Motor Deficit Testing

Rotarod testing (Panlab Rotarod LE 8200, Harvard instruments) is a routinely used behavioural assessment for neuromuscular coordination—or motor deficits (Deacon, 2013. Prior to assessment, mice were familiarized with the rotarod apparatus in a pre-training session (24 hours beforehand), where they were exposed to the apparatus and placed on the rod at a low rotation speed (4 rpm) for 5 minutes. Mice that fell off the rod were placed back on the rod until the 5-minute duration was completed. On the day of the experiment, individual mice were placed on the rotating rod, and the timer was started upon contact. The rod spun from a base speed of 4 rpm up to 40 rpm over a period of 1 minute, with trials being terminated after the mouse fell off the rod. A trial was considered null if the mouse voluntarily jumped off the rod, and the trial was repeated. The latency (time) each mouse remained on the rotarod was recorded. Two trials were recorded with a minimum of 5 minutes between them for each mouse. Final values were taken as an average of the two trials.

### Grip Strength

Grip strength was measured using a portable scale with a grid attached to allow the mouse to grip. All measurements of force were taken as kilos/grams of pressure. Briefly, a mouse was held by the tail, and lifted to a force transducer apparatus until it grabbed on to the handle. Once the mouse consistently grabbed the handle when placed near the apparatus, it was left for at least 5 minutes before assessment. Three assessment trials were recorded, whereby the mouse gripped on to the apparatus, and was pulled gently until it lost its grip. The maximum force as recorded by the apparatus from the three trials was recorded and used for analysis.

### RBC-EV Preparation

### Incubation

Whole mouse blood in EDTA was purchased from Applied Biological Products Management (MSBX 0005; 5 ml tubes). Whole blood was spun at 1500×g for 10 minutes to separate the plasma, buffy coat, and red blood cells. Plasma and buffy coat were removed via pipetting, and the remaining red blood cells were diluted 1:10 in 1× phosphate buffered saline (PBS) (Gibco; pH 7.2). Red blood cell (RBC) suspension was passed through a leukocyte depletion filter (Sterile Acrodisc® WBC syringe filter with Leukosorb Membrane, 25 mm; Pall) to remove contaminant leukocytes. RBC suspension was then incubated in T25 flasks supplemented with 100× N1 Medium Supplement (Sigma; N6530) at 1:100 dilution, and superoxide dismutase (SOD) (Worthington Biochemicals; LS003540) at 1:500 dilution (5 μg/ml; the final working concentration of SOD). RBC suspension with supplements was incubated at 37 C on a shaker incubator for 18 hours.

### Supplementation

Trans-resveratrol (Sigma PHR2201-200 MG), Kaempferol (Sigma, 60010-25 MG) and L-Glutathione reduced (Sigma, G4251-1G) were added in increasing doses to RBC for 18 h at 37 C as per the incubation paradigm above and compared to RBC (PBS), RBC (N1/SOD), and pre-treatment (freshly isolated RBC from whole mouse blood in EDTA, no supplement or incubation).

### RBC Health Measurements

Post incubation, the RBC suspension was transferred to 15 mL falcon tubes and spun at 600×g for 20 minutes to separate RBC from extracellulae vesicles (EVs) in suspension (Usman et al, 2018). To determine the effect of incubation on the health of RBCs, the 600× g pellet was resuspended in 1×PBS (Gibco; pH 7.2) in 1:1000 and 1:10000 dilution to perform qualitative and quantitative assessments. 200 μL of pellet suspension was applied to a glass hemocytometer (Westlab, product number 071301-9877) and the number of RBCs in the large centre square were counted to determine total cell number. Zeiss Axiovert 200 microscope was used to take images of RBCs at 4× and 20× magnification and images were processed using ImageJ software (version 2.1.0). RBC abnormality percentage was determined by studying RBC membrane and classifying them based on RBC size (normal, microcyte and macrocyte) and membrane shape variation (normal, burr cell, tear drop, sickle cell, ovalocyte and schistocyte), all statistical analysis was performed on GraphPad Prism 9.

Calcein-AM (Thermo Fisher, Cat #C1430) and Annexin V (BD Biosciences, Cat #563973) staining was performed to assess cell viability of RBC pre and post treatment/incubation. Calcein-AM was prepared as a stock solution of 10 mM in dimethylsulfoxide and a working solution of 100 μM in PBS buffer, pH 7.4 was prepared as required. RBC, 2× 105 in 200 μl PBS, were incubated for 45 minutes with calcein-AM working solution (final concentration in calcein-AM-5 μM) at 37 °C (dark). Cells were then isolated by centrifugation (1000×g at 4 °C for 5 min), resuspended in 0.5 ml of Annexin V binding buffer containing (145 mM NaCl, 7.5 mM KCl, 2 mM CaCl2, 10 mM glucose, 50 mM HEPES pH 7.4, 0.5% BSA), and incubated with 5 μl PE-annexin-V for 15 min at room temperature in the dark. Cells were analyzed for biparametric histograms FL1 (calcein) versus FL2 (PE-annexin-V) using ImageStreamX Mk II (Amnis Corporation, Seattle, WA, USA) and the BD LSRFortessa II (BD Biosciences, San Jose, California). RBC experimental permeabilization as a control of dying cells was readily made with saponin according to Jacob et al, 1991). All experiments were carried out in triplicate.

### EV Isolation

Following the 600× g spin, the RBC supernatant was transferred to 15 mL falcon tubes and spun at 1600×g for 15 minutes and 3200× g for 10 minutes, excluding the pellet at each spin step by transferring the supernatant to a fresh tube. The resulting RBC suspension S1 was transferred to ultracentrifuge tubes (Beckman Coulter Ultra-Clear Thinwall Tubes 13.2 ml; pack of 50) and spun using an ultracentrifuge at 10,000×g for 35 minutes. The resultant pellet P2 was discarded and the supernatant S2 was transferred to fresh ultracentrifuge tube and respun at 100,000×g for 1 hour 30 minutes. P3 containing RBC-EV was then collected, washed in PBS by pipetting, and respun at 100,000×g for 1 hour 35 minutes (Usman et al, 2018)). Finally, P4 (RBC-EV pellet) was resuspended in 50-500 μL (downstream experiment dependent) and frozen at −80 C until use.

### SOD and Hemoglobin Activity Assays

Snap-frozen aliquots of RBC and RBC EV were thawed and lysed in ice cold ultrapure water and PBS+0.1% Triton in 1:150 and 1:4 dilutions respectively. The samples were left on ice for 20 minutes to ensure complete lysis and spun down at 10,000×g for 15 minutes. The supernatant was used for SOD and hemoglobin activity assays. SOD activity of RBC and RBC-EV with and without N1, SOD and N1+SOD was measured using Superoxide Dismutase (SOD) Colorimetric Activity Kit as per manufacturer's instructions (Cat no: EIASODC, Thermo Fisher Scientific) and compared to no supplement controls. Similarly, hemoglobin activity was assessed using a hemoglobin colorimetric assay (Abcam, ab234046) in accordance with manufacturer protocols.

### EV Characterisation

The concentration and size distribution of human RBC-EV was measured using nanoparticle tracking analysis on a ZetaView x30 QUATT (Particle Metrix GmbH) on a 488 nm excitation laser following calibration with 100 nm polystyrene latex reference standards (Applied Microspheres, Netherlands). The following parameters were used: (sensitivity: 80, shutter: 100, minimum brightness: 30, minimum size area: 10, maximum size area: 1000, frame rate 30 frames/second). The RBC-EV were diluted 1:100,000 in sterile 1×PBS. 1 cycle of 11 positions was captured across the flow cell and averaged. The mean concentration value for each sample, in particles/mL, and mean and mode size (nm) was exported to Prism 9 for plotting and statistical analysis.

### RBC-EV Administration

To test safety and therapeutic efficacy of RBC-EV, mice were administered with RBC-EV both locally and systemically. Mice were either injected with RBC-EV (SOD/N1), RBC-EV (SOD), RBC-EV (N1), RBC-EV (PBS) in 1 μL, or 1 μL PBS (SOD/N1), PBS (SOD), PBS (N1) or PBS as controls. In some experiments, RBC-EV or PBS were labelled with SYTO RNASelect to fluorescently label EV RNA, as described below, before administration. Mice from the same litter were used in each experiment for comparison and were randomly assigned across treatment groups.

### Intravitreal Injections

Mice were first anaesthetised with an intraperitoneal injection of Ketamine (100 mg/kg) and Xylazil (10 mg/kg) and then 1% w/v Atropine was applied to the ocular surface of each eye to dilate the pupils. Mice were kept on a heated mat while anaesthetised to ensure maintenance of the mouse's body temperature. A string loop was first tied around the eye of the mouse to lift the eye from its socket and allow for easier access to the injection site. The surface of the eye was swabbed with 10% w/v povidone-iodine antiseptic liquid. A 33G needle was used to make a pilot hole at the injection site, approximately 1 mm posterior to the temporal limbus. A 10 μL NanoFil syringe, with a 34G needle was used to inject 1 μL (2.0×10⁹ EV) of each EV mixture or control solution through the previously made pilot hole into the vitreous of the eye, angled toward the optic nerve, with the aid of a stereo microscope. After injection, the eye was swabbed with 1% Chlorsig to prevent bacterial infection and GenTeal™ Gel to prevent eye dryness. To aid recovery, animals were intraperitoneally injected with ReversaMed (1 mg/kg). Animals recovered for around 24 hours in dim conditions before being placed into photo-oxidative damage for 5 days (degeneration paradigm), or were kept in standard housing conditions for 7 days (safety assessment).

### Intraperitoneal Injections

Mice were injected via intraperitoneal injection at 2.0×10¹¹ EV/100 μL dose daily using the regimen outlined in the figures.

### Intracranial Injections

RBC-EV at a dose of 2.0×10⁹ EVs/μL (1 μL bolus) or vehicle control (PBS) will be administered during intracranial delivery of 6-OHDA/vehicle using the regimen outlined in the figures.

### Extracellular Vesicle Characterisation

### Cryogenic Electron Microscopy

CryoEM was used to visualise and image RBC-EV for size measurement and characterisation purposes. RBC-EV samples in PBS were applied to a glow-discharged (PELCO easiGlow™ Glow Discharge Cleaning System) 300 mesh EM grid with lacey carbon for vitrification at 80-90% humidity, in room temperature. Any excess sample present was removed by blotting with filter paper. Loaded grids were placed into liquid ethane (kept in equilibrium with solid ethane) using a Leica EMGP2 Automatic Plunge Freezer (Centre for Advanced Microscope facility, JCSMR). This grid was then stored in liquid nitrogen until further use. A JEOL JEM-F200 microscope was used to image EV at 200 kV voltage and 16,000× magnification. Images were processed using ImageJ. All EVs visible across 10 images were measured for size (in nanometres) and exported to GraphPad Prism 9 for plotting and statistical analysis.

### NanoSight Characterisation

The concentration and size distribution of RBC-EV was measured using nanoparticle tracking analysis on a NanoSight NS300. The RBC-EV were diluted 1:10,000 in sterile 1×PBS to achieve a particle per frame value between 20 and 100. A constant flow of the sample was provided by a syringe pump set at a speed of 35 (equivalent to ~3.1 μL/min) (Wooff et al, 2020). Nine 30-second-long videos were captured for each sample. The detection threshold, set to between 4 and 5, was not altered between measurements. The mean concentration value for each sample, in particles/mL, was exported to GraphPad Prism 9 for plotting and statistical analysis.

### Western Blot Analysis

Total lysates were extracted from cells and RBC-EV by incubating with RIPA buffer supplemented with 1:100 protease inhibitor cocktail (Sigma-Aldrich, MO, United States). An additional homogenisation step was applied for retinal tissue samples. 10-20 μg of protein lysates/well were separated on 4-12% polyacrylamide gels (Thermo Fisher Scientific, MA, United States) at 100 V for 60 minutes and transferred to a Nitrocellulose membrane (Bio-Rad, CA, United States) using a Power Blotter semi-dry system (Thermo Fisher Scientific, MA, United States) at 20 V for 15 minutes. Membranes were washed in PBS-Tween (0.01%; PBS-T), blocked in Pierce™ Clear Milk Blocking Buffer (Thermo Fisher Scientific, MA, United States) for 1 hour and then incubated overnight at 4 °C with primary antibodies TSG101 (1:1000, ab30871, Abcam, Cambridge, United Kingdom), Alix (1:1000, EPR23653-32, ab275377, Abcam, Cambridge, United Kingdom), Calnexin (1:1000, ab22595, Abcam, Cambridge, United Kingdom) or GAPDH (1:2000, G9545-100UL, Sigma-Aldrich, United States).

Following three washes in PBS-T, blots were incubated in appropriate secondary antibodies, HRP-conjugated Goat Anti-Rabbit IgG (H+L) (1:1000, 170-6515, Bio-Rad, CA, United States) or Goat-anti-Mouse IgG (1:1000, 170-6516, Bio-Rad, CA, United States) for 2 hours at room temperature. Membranes were washed in PBS-T and developed for 2 minutes with Clarity™ Western ECL Substrate (Bio-Rad, CA, United States). Imaging was performed using a ChemiDoc™ MP Imaging System with Image Lab™ software (Bio-Rad, CA, United States).

### Retinal Tissue Analysis

### Tissue Collection and Preparation

Animals were ethically euthanised with CO₂ following PD. The superior surface of the left eye was marked and enucleated, then immersed in 4% paraformaldehyde for 3 hours. Eyes were then cryopreserved in 15% sucrose solution overnight, embedded in OCT medium (Tissue Tek, Sakura, Japan) and cryosectioned at 12 μm in a parasagittal plane (superior to inferior) using a CM 1850 Cryostat (Leica Biosystems, Germany). To ensure accurate comparisons were made for histological analysis, only sections containing the optic nerve head were used for analysis. The retina from the right eye was excised through a corneal incision and placed into RNAlater solution (Thermo Fisher Scientific, MA, United States) at 4 °C overnight and then stored at −80 C until further use.

### Immunolabelling

Immunohistochemical analysis of retinal cryosections was performed as previously described (Rutar et al, 2015). Fluorescence was visualised and images taken using a laser-scanning A1⁺ confocal microscope at 20× and 40× magnification (Nikon, Tokyo, Japan). Image panels were analysed using ImageJ V2.0 software and assembled using Illustrator software (Adobe Systems, CA, United States).

### Immunohistochemistry and Analysis

Immunolabelling for IBA1 (1:500, 019-19741, Wako, Osaka, Japan), a marker of microglia and macrophage immune cells was performed as previously described (Rutar et al, 2015). Retinal cryosections were stained with the DNA-specific dye bisbenzimide (BBZ; 1:10000, Sigma-Aldrich, MO, United States) to visualise the cellular layers. The number and morphology (ramified vs amoeboid) of IBA1⁺ cells were counted across the superior and inferior retina using two retinal sections per mouse. Using ImageJ V2.0 software, intensity analysis was performed 0.5 mm superior to the optic nerve and calculated as a relative intensity from controls.

### TUNEL Assay

Terminal deoxynucleotidyl transferase (Tdt) dUTP nick end labelling (TUNEL), was used as a measure of photoreceptor cell death. TUNEL in situ labelling was performed on retinal cryosections using a Tdt enzyme (Cat #3333566001, Sigma-Aldrich, MO, United States) and biotinylated deoxyuridine triphosphate (dUTP) (Cat #11093070910, Sigma-Aldrich, MO, United States) as previously described (Natoli et al, 2010). Images of TUNEL staining were captured with the A1⁺ Nikon confocal microscope at 20× and 40× magnification. The total number of TUNEL⁺ cells were counted including both the superior and inferior retina using two retinal sections per animal.

To further quantify photoreceptor survival, the thickness of the outer nuclear layer (ONL) on retinal cryosections was determined by counting the number of nuclei rows (photoreceptor cell bodies) in the area of retinal lesion development (1 mm superior to the optic nerve head). Photoreceptor cell row quantification was performed five times per retina using two retinal cryosections at comparable locations per mouse.

### Brain Tissue Analysis

### Tissue Collection and Preparation

Following behavioural testing, mice were individually sacrificed via cervical dislocation, and the brain was immediately removed and placed into ice-cold PBS. An incision was made throughout the left cortex of the brain to ensure the hemispheres were not mixed up throughout the slicing procedure. The brains were then sliced in ice-cold PBS coronally using a vibratome (Leica 1200s; 1 mm thick, 0.75 mm amplitude, speed 1 mm/sec), and the striatum was carefully dissected in ice cold PBS from each slice using fine-pointed forceps. The control and experimental hemisphere were collected in separate tubes. Once the whole striatum was collected, the tissue was flash-frozen on dry-ice and placed at −80 degrees until western blotting could be completed.

### Western Blot Analysis

A Bradford assay Rapid gold BCA assay (ThermoFisher, A55860) was conducted on each sample to confirm protein concentration. Samples were placed in 4× Laemmli sample buffer (BioRad, 1610747) with 5% 2-Mercaptoethanol for reduction. Samples were denatured at 95 degrees on a heat block for 5 minutes, and then loaded into wells of Bolt™ Bis-Tris Plus Mini Protein Gels, 4-12% (Invitrogen, NW04122BOX). Gels underwent 120V electrophoresis in 1×MES SDS running buffer (Invitrogen, NP0002) for 1 hour at room temperature. Gels were then transferred onto nitrocellulose membrane (BioRad, #1620112, 0.2 μm pore size), running at 100V at 4 degrees for 60 minutes.

Membranes were then blocked for one hour in 5% skim milk powder in PBS-T (0.05% Tween) with constant rocking. Primary antibodies were then incubated overnight at 4 degrees (IBA1, Abcam, AB5076; Beta-Actin, Cell signalling, 8457S; Tyrosine Hydroxylase, ThermoFisher, OPA1-04050) diluted 1:1000 in PBS-T (2.5% skim milk, 0.05% tween). Membranes were then washed at room temperature in PBS-T 30 minutes, and then secondary antibodies were added for 1 hour (anti-Rabbit 41460; anti-Goat AP163P; 1:10,000 dilution, PBS-T w/2.5% skim milk, 0.05% Tween). Membranes were then washed in PBS-T.

Membranes were developed using the clarity western ECL substrate (BioRad #1705061), which was incubated for 5 minutes. Membranes were then imaged using the Chemidoc MP imaging system (BioRad).

All analysis was completed by measuring band intensity using the measure function in ImageJ. All signals were normalised back to the intensity of the house-keeping protein Beta-Actin, and the experimental side was normalised to the control side.

### MiRNA Loading RBC-EV

### Electroporation

Electroporation of RBC-EV was performed using a Gene Pulser II (BioRad) electroporation system, exponential program at a fixed capacitance of 125 μF with 0.2 cm cuvettes. For optimisation, 1 to 2×10\hat{ }11 RBC-EV were diluted either in 1×PBS (Gibco 7.2 pH), Opti-MEM™ Reduced Serum Medium, GlutaMAX™ Supplement (Thermo Fisher Scientific, MA, United States) or in Gene Pulser Electroporation Buffer (BioRad) mixed with 2-8 μg of microRNA to a total volume of 200 μl. 200 μl EV mixture was added to each cuvette and incubated on ice for 10 minutes. Electroporation was tested at different voltages: 100-450 V. Aggregates of RBC-EV formed during electroporation were dissolved by vigorous pipetting. To quantify the electroporation efficiency, electroporated RBC-EV were lysed and miRNA quantified using Qubit™ microRNA assay kit.

### Passive Loading

RBC-EV (1 to 2×10\hat{ }11) were diluted in 1×PBS (Gibco 7.2 pH), OptiMEM™ Reduced Serum Medium, GlutaMAX™ Supplement (Thermo Fisher Scientific, MA, United States) or in Gene Pulser Electroporation Buffer (BioRad) mixed with 2-8 μg of microRNA to a total volume of 200 μl. 200 μl EV mixture was incubated at 4 or 37 °C for 1, 4 and 18 hours to determine the optimal condition for passive loading. To quantify the passive loading efficiency and compare it to electroporation, RBC-EV were lysed, and miRNA quantified using Qubit™ microRNA assay kit.

miRNA Quantification

The actual concentration of miRNA encapsulated in RBC-EV was measured using the Qubit™ microRNA assay kit. The RBC-EV-miRNA complex was first lysed by adding 5 μL of the complex into a SDS buffer containing 90 μL of sterile 1×PBS and 3 μL of 0.5% SDS, releasing the incorporated miRNA, a protocol adapted from (Lamichhane and Jay, 2018). The miRNA-SDS solution was mixed well and incubated at 85 C for 15 minutes, also mixing well halfway through. The kit was then used to quantify miRNA as per the manufacturer's instructions. 1 μL of the miRNA-SDS solution was added in a Qubit™ assay tube to 199 μL of the Qubit™ working solution, made by diluting the provided Qubit™ microRNA reagent 1:200 in the Qubit™ microRNA buffer. The tube was vortexed for 5 seconds and then incubated at room temperature for 10 minutes. The concentration of the samples was read and calculated using the Qubit™ 3 Fluorometer, calibrated with the provided standards.

### In Vitro Experiments

Murine photoreceptor-derived 661W cells (kindly gifted by Dr. Muayyad R. Al-Ubaidi, University of Houston (Al-Ubaidi et al, 1992), human Müller-like MIO-M1 cells (Moorfield's Institute of Ophthalmology) (Limb et al, 2002), human RPE-like ARPE-19 cells (ATCC) (Dunn et al, 1996) and Murine microglia-like BV2 cells (Accegen) (Blasi et al, 1990) were used for in vitro experiments. Cells were cultured in growth media in flasks in a humidified incubator at 37 C with 5% CO₂. Cells were passaged every 3-5 days by trypsinisation with 0.25% trypsin, with the use of Nunc™ Cell Scrapers for MIO-M1 and ARPE-19 cells.

### RBC-EV Labelling

### SYTO™ RNASelect™ Green Fluorescent Cell Staining

To determine RBC-EV uptake in vivo and in vitro, RBC-EV were labelled using the SYTO™ RNASelect™ Green Fluorescent Cell Stain, a cell-permeant RNA-specific stain. The stain was first diluted 1:5 in DMSO (#ICN19141880, Thermo Fisher Scientific, MA, United States) to give a 1 mM DMSO stock. The RBC-EV stock was diluted in sterile 1×PBS to a total volume of 100 μL. 1×PBS was used as a control for background staining. 1 μL of the DMSO stock was then added to the diluted RBC-EV, mixed thoroughly, and incubated at 37 C for 30 minutes. At approximately halfway, the solutions were mixed to ensure the dye did not settle at the bottom of the tube. Afterwards, the solutions were added to Amicon™ 50 kDa MWCO filter units (UFC505024, Merck) pre-rinsed with UltraPure™ distilled water, and spun at 5000×g for 20 minutes to remove excess unbound dye. The solutions were further washed thrice by adding 500 μL of sterile 1×PBS and spinning the columns at 10,000×g for 10 minutes each time. The solutions were then made up to the same volume using sterile 1×PBS and immediately added to cells. Cells were placed in an incubator set to 37 C with 5% CO₂ and fitted with the IncuCyte™ ZOOM system. Phase images, to visualise cell morphology, and fluorescent images in the green channel (with an excitation wavelength of 440-480 nm and an emission wavelength of 504-544 nm), to visualise the labelled RBC-EV, were taken every 2 hours at 10× magnification for 48 hours. The fluorescently labelled RBC-EV or PBS-only control solutions were also administered to mice via intravitreal injection (locally), or intraperitoneal injection (systemically) as mentioned above.

### In Vitro Experiments

### 661W Cell Culture

Murine photoreceptor-derived 661W cells (kindly gifted by Dr. Muayyad R. Al-Ubaidi, Department of Biomedical Engineering, University of Houston, Houston, TX, United States) (Al-Ubaidi et al., 2013), were used for in vitro experiments at passage 1-5. The authenticity of the cells was validated by short tandem repeat analysis (CellBank, Sydney, Australia). Cells were cultured in growth media (Dulbecco's Modified Eagle Medium (DMEM; Sigma-Aldrich, MO, United States) supplemented with 10% fetal bovine serum (FBS; Sigma-Aldrich, MO, United States), 6 mM L-glutamine (Thermo Fisher Scientific, MA, United States) and antibiotic-antimycotic (100 U/ml penicillin, 100 μg/ml streptomycin; Thermo Fisher Scientific, MA, United States)). Cells were maintained and all incubation steps were performed in dark conditions in a humidified atmosphere of 5% CO₂ at 37 °C, unless otherwise stated. Cells were passaged by trypsinization every 3-4 days.

To deplete FBS of EV, the serum was centrifuged (200,000×g, at 4 °C for 18 h) using a Beckman Coulter Optima XE-100 Ultracentrifuge (Beckman Coulter, CA, United States), with a SW41Ti rotor (Beckman Coulter, CA United States) and the supernatant used as FBS supplement in all EV collection experiments.

N1 Medium Supplement (Sigma; N6530) at 1:100 dilution, and superoxide dismutase (SOD) (Worthington Biochemicals; LS003540) at 1:500 dilution were added to 661w for 18 hours before cell supernatant was collected. EV were isolated using differential ultracentrifugation as previously published (Wooff et al., 2020).

### In Vitro Photo-Oxidative Damage

Following 18 hours N1/SOD incubation, cells were exposed for 2 h to 15,000 lux light (2.2 mW/cm²; irradiance measured with PM100D optical power meter, Thorlabs, NJ, United States) from two white fluorescent lamps (2×10W T4 tri-phosphor 6500K daylight fluorescent tubes; Crompton, NSW, Australia (Lu et al., 2017; Fernando et al., 2018). Control cells were completely wrapped in aluminum foil with six small incisions to allow air/gas exchange.

### Quantitative Proteomics

### Protein Isolation

Frozen aliquots of RBC and RBC-EV+/−supplement groups (N1, SOD and N1/SOD) were sent to Australian Proteome Analysis Facility (APAF), Macquarie University, NSW, Australia for protein isolation and digestion and LC-MS/MS Tandem Mass Spectrometry using Orbitrap Exploris (Thermo Fisher Scientific) and NanoLC Vanquish Neo UHPLC (Thermo Fisher Scientific) systems.

### Bioinformatics and Analysis

Preprocessing: The initial processing of protein intensity data began with the application of a base-2 logarithmic transformation. Proteins presenting with greater than two missing values within any single experimental group were subsequently excluded from the expression matrix to ensure data integrity.
Imputation of Missing Values: For the remaining proteins that exhibited missing values, an imputation strategy was employed. This involved the generation of random values from a Gaussian distribution, which were anchored at the minimal value requisite for imputation for each respective protein. This minimal value equalled the 0.01 quantile of the observed data for each sample, representing a robust estimate of low expression values. The standard deviation used in the Gaussian distribution equalled the standard deviation of the sample requiring imputation.
Data Normalization: Post-imputation, data normalization was carried out using a variance stabilization transformation (VST). The normalize_vst function within the R DEP package was utilized for this purpose. This function adjusted the count data, by normalization factors, to produce a matrix with values that are approximately homoskedastic. Thus, this transformation ensured that the variance of the data remained constant across the range of mean values.
Differential Expression Analysis: The processed data were then subjected to differential expression analysis following the limma pipeline. This entailed the use of the ImFit function to fit linear models for each protein, followed by the application of empirical Bayes smoothing of the standard errors through the eBayes function. Finally, the top Table function was used to tabulate the most differentially expressed proteins. The limma pipeline is a well-established methodology in the field of bioinformatics for identifying differentially expressed genes or proteins with precision and reliability.
Enrichment analysis: Pathway analysis was conducted through gene set enrichment analysis (GSEA) utilizing the fgsea function. The method employed t-statistics as the ranking metric to prioritize genes, with the C2 canonical pathways and C5 gene ontology database from the Molecular Signatures Database (MsigDB) serving as the reference gene sets. Additionally, overrepresentation analysis (ORA) was carried out using the Enrichr platform to identify significantly enriched pathways.

### RNA Sequencing

### RNA Isolation and Quality Assessment

Small RNA sequencing began with the lysis of RBC-EV in 300 μL of lysis buffer, followed by the addition of 30 μL miRNA homogenate, and incubated on ice for 10 minutes. Subsequently, 330 μL Acid-Phenol: Chloroform was added, and the mixture was vortexed for 60 seconds. Centrifugation at 10,000×g for 5 minutes facilitated phase separation. The aqueous phase was transferred and mixed with 1.25× volume of ice-cold absolute ethanol for total RNA isolation, or with ⅓× volume for small miRNA enrichment. The mixture was then passed through a purification column. The column was washed with 700 μL miRNA wash solution #1 and twice with 500 μL wash solution #2, then spun dry. RNA was eluted with heated solution (95 °C), collected into 30 μL, and stored at −80 °C. RNA quality and concentration were measured using a Nanodrop ND-1000 spectrophotometer and Agilent 2100 Bioanalyzer.

### Library Preparation

The library preparation for high-throughput sequencing of RNA from RBC EV was carried out at the Biomolecular Research Facility (JCSMR, ANU). Libraries were synthesized using the Capture and Amplification by Tailing and Switching (Diagenode). Libraries were sequenced on the Illumina NovaSeq 6000 acquiring at least 10 million 50 bp single-end reads per sample.

### Bioinformatics and Analysis

Preprocessing: Indexes, adapters, and template-switching oligonucleotides were removed with cutadapt using the following unix shell scrip as per product instructions:
Alignment: subread-align was used of reads to the mm10 mouse genome specifying the following unix shell script:
subread-align -t 1-i/mouse mm10 index -n 35 -m 4-M 3 -T 10 -I 0--multiMapping -B 10 -r miRNA_reads.fastq -o result.sam
   Quantification: featureCounts was ran for quantifying miRNA alignments using the following unix shell script:
featureCounts -t miRNA -g Name -O -s 1 -M -a/data/human/miRBase/mmu.gff3 -o miR_counts.txt result.sam
   Differential Expression Analysis: The following command sequence voom-->ImFit-->ebayes-->topTable was used for assessing differential gene expression data in Rstudio using limma package.
   Voom: Voom function is part of the limma package and is used to transform count data from RNA-seq to log 2-counts per million (log-CPM), which are suitable for linear modeling. voom estimates the mean-variance relationship of the log-counts, assigns a weight to each observation, and generates a ‘voom’ object that includes the expression data with the weights. These weights are used in subsequent modelling steps to account for the heteroscedasticity (non-constant variance) inherent in count data.
   ImFit: Once the voom transformation is complete, the ImFit function fits a linear model to each gene (or transcript). This function accommodates the design matrix of the experiment, which specifies the different conditions the RBCEV were subjected to. ImFit uses the weighted data from the voom step to fit the model, taking into account the design of the experiment.
   ebayes: After fitting the linear models, empirical Bayes moderation is applied using the ebayes function. This step borrows information across all genes to obtain more precise estimates of gene-wise variances and, in turn, more stable inference for differential expression.
   topTable: The final step involves summarizing the results using the topTable function. This function ranks genes based on the results of the ebayes by the p-value or adjusted p-value and fold change. Genes with p-values <0.05 and log fold changes >0.5 were deemed biologically relevant.

### RBC-EV Viability and Inflammatory Profiling

RBC-EV+/−supplement groups (N1, SOD, N1/SOD) in frozen −80 C aliquots were sent to CruxBioLabs (VIC, AUS) for independent validation of safety/toxicity and anti-inflammatory properties using control and LPS stimulated human peripheral bone mononuclear cells (PBMC).

### Flow Cytometry Analysis in RBC-Enriched Fraction

To assess the proportion of red blood cells (RBCs) after plasma removal and leukocyte depletion, RBCs were diluted 1:10 in PBS with 1×N1 medium supplement and SOD. Then, 10 μl of this diluted sample was combined with each of the following antibody preparations: TER119-PE-Cy7 (BD Biosciences, 557843), CD41-FITC (BD Biosciences, 553848), CD71-eFluor450 (LifeTech, 48-0711-82), CD45-APC-Cy7 (LifeTech, 47-0451-82), and a no-antibody control, and incubated on ice for 30 minutes. 200 μl of FACS buffer (PBS, 0.5% BSA) was added to the samples and centrifuged at 1500×g for 5 minutes to pellet the cells. The pellet was resuspended in 200 μl of FACS buffer and analysed on an LSR-II, recording 1 million RBC events to identify populations of RBCs, platelets, leukocytes, and reticulocytes.

### Example 2: Extracellular Vesicles can be Isolated from Mouse Red Blood Cells

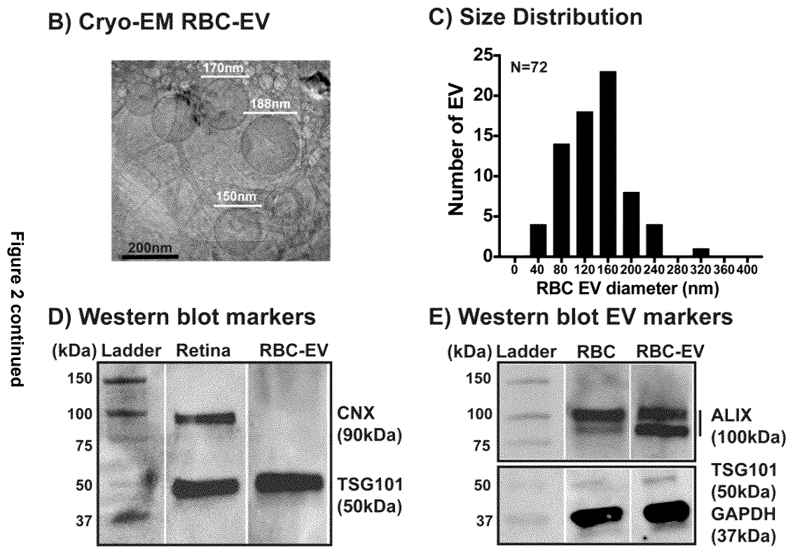

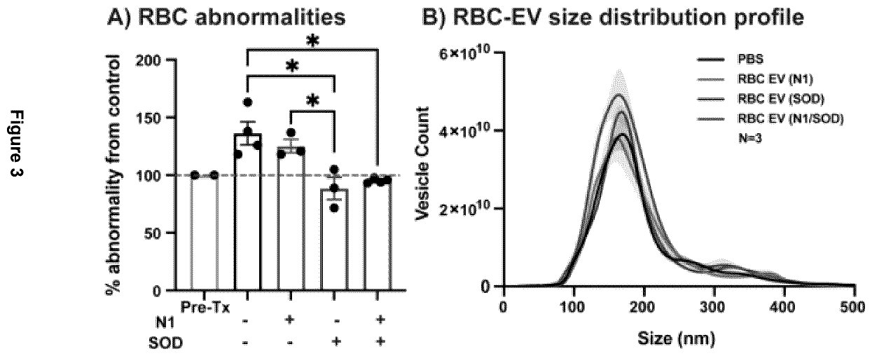

Red blood cell-derived extracellular vesicles (RBC-EV) were isolated from mouse whole blood using a novel experimental paradigm. Following plasma removal and leukocyte depletion, RBC were incubated overnight at 37 C in a 1:10 dilution of PBS with, or without culture supplements N1 and/or SOD. Serial differential ultracentrifugation was used to isolate small EVs from the RBC suspension (pellet 4; P4). (FIG. 2A). RBC-EV were characterised for morphology and size using cryogenic electron microscopy (cryo-EM), displaying a rounded shape (FIG. 2B), and a size distribution from 40 nm to 320 nm, peaking at 160 nm (FIG. 2C). Western blot was performed on retinal cell lysates and RBC-EV lysates to identify cell contaminant markers and EV markers. While a strong band at 90 kDa was present for cellular marker Calnexin (CNX) in retinal lysates, there was no expression shown in RBC-EV. Both retinal and RBC-EV lysates had EV marker Tumour susceptibility gene 101 (TSG101) bands at 50 kDa. (FIG. 2D). To identify EV marker enrichment, EV marker proteins TSG101 and ALIX were compared on RBC and RBC-EV lysates, relative to reference protein GAPDH (37 kDa). Both TSG101 (50 kDa) and ALIX (100 kDa) were found at higher expression in RBC-EV compared to RBC (FIG. 2E). Taken together these results demonstrate that EVs can be isolated from RBC and display classical EV markers and undetected contamination.

### Example 3: N1 and SOD Supplementation Improves RBC Health and RBC-EV Quality

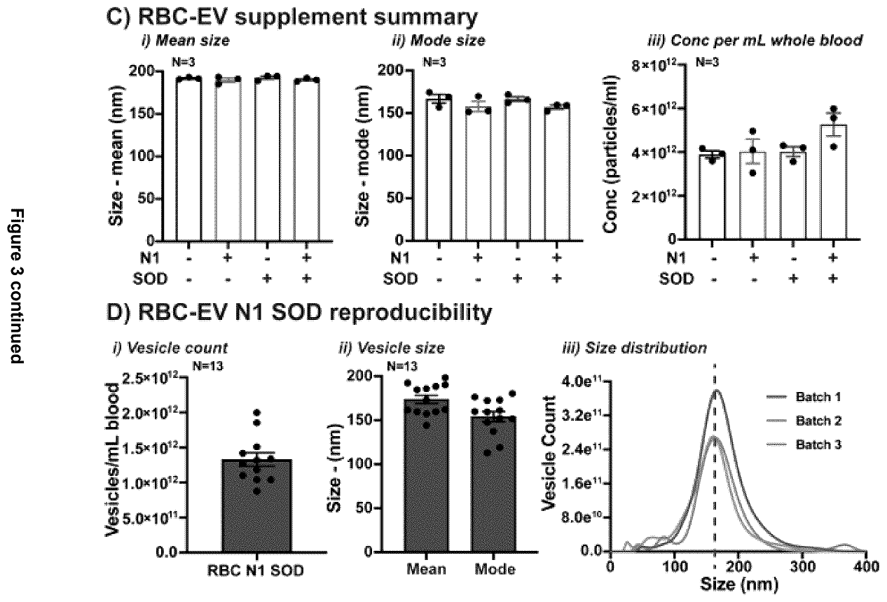

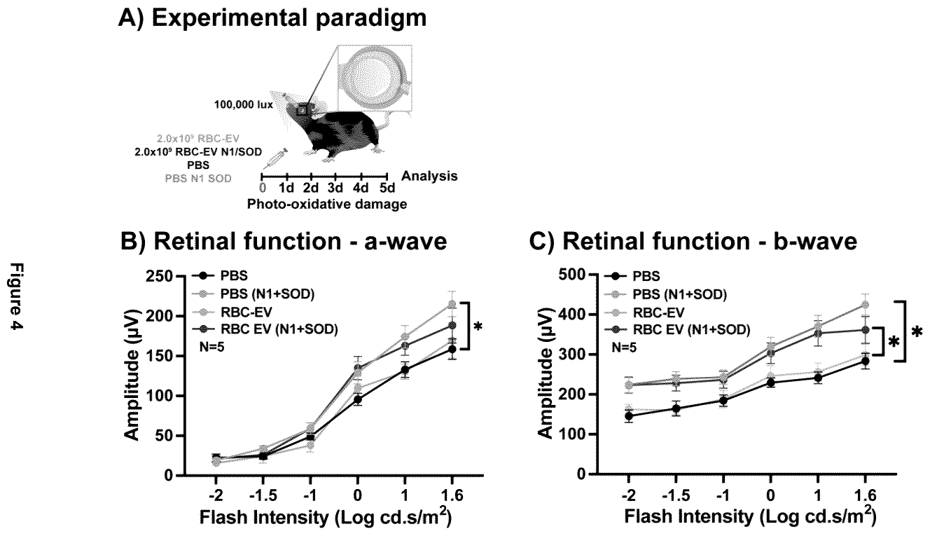

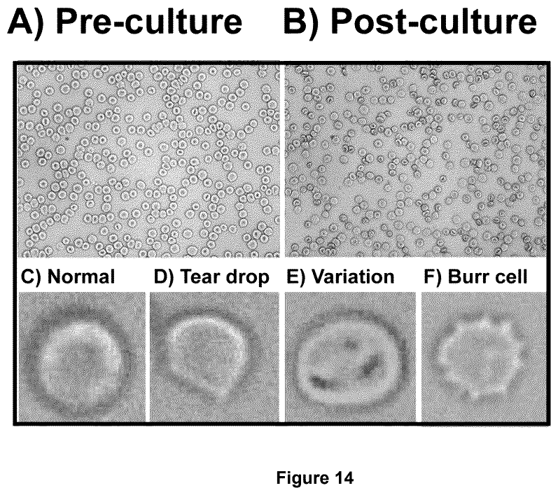

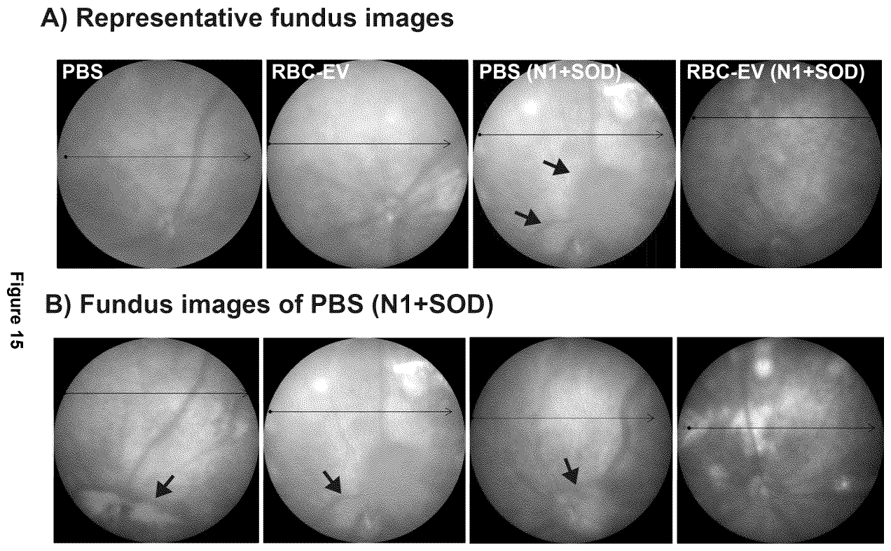

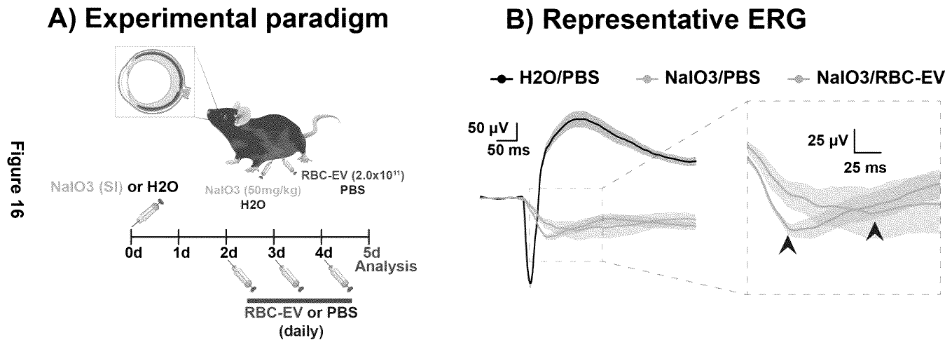

To improve RBC incubation and RBC-EV quality, N1 media supplement and antioxidant SOD were added individually and in combination to RBC overnight incubation paradigm and compared to RBC-PBS. RBC abnormalities (tear drop shape, burr cell) were quantified before and after overnight incubation at 37 °C (FIG. 14). Compared to fresh blood (pre-treatment; Pre-Tx), RBC incubated in PBS alone and in N1 supplement had higher abnormalities, while RBC with SOD, or with N1 and SOD combination had significantly lower abnormalities compared to RBC-PBS and RBC-N1 groups (FIG. 3A, P<0.05). The size distribution profile of RBC-EV was also measured for each of the preparations using Nanotracking analysis (Nanosight NS300), showing a uniform size distribution with a single peak EV for N1, SOD and N1/SOD groups, while RBC-PBS alone produced a double peak EV as per our measures, the reproducibility of this incubation was further tested. On average, the number (FIG. 3D (i)), mean and modal size (FIG. 3D (ii)) and size distribution profile (FIG. 3D (iii)) of RBC-EV from N1/SOD incubation remained consistent across batches of blood, demonstrating a reproducible and reliable incubation and isolation paradigm.

### Example 4: N1 and SOD Combination Supplementation Provided Neuroprotective Properties in a Photo-Oxidative Damage-Induced Retinal Neurodegeneration Model

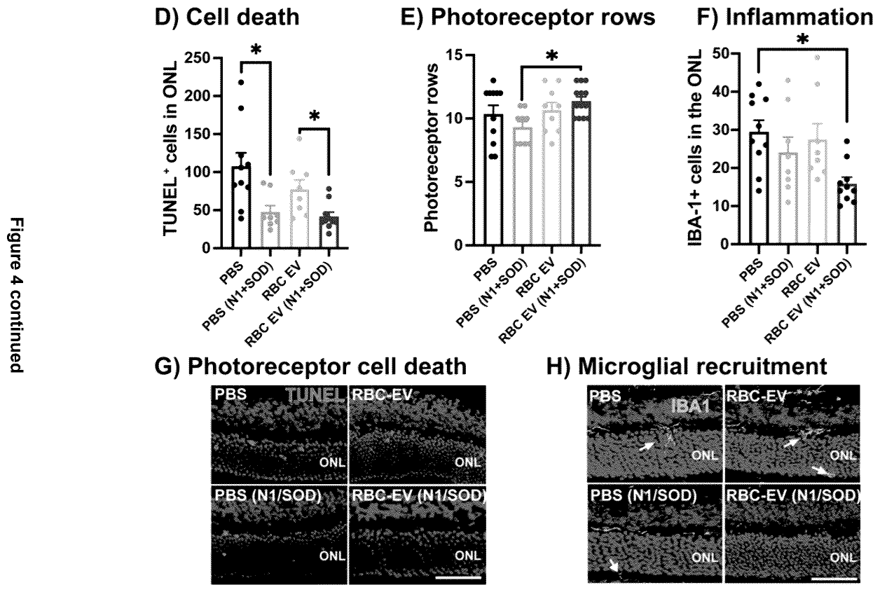

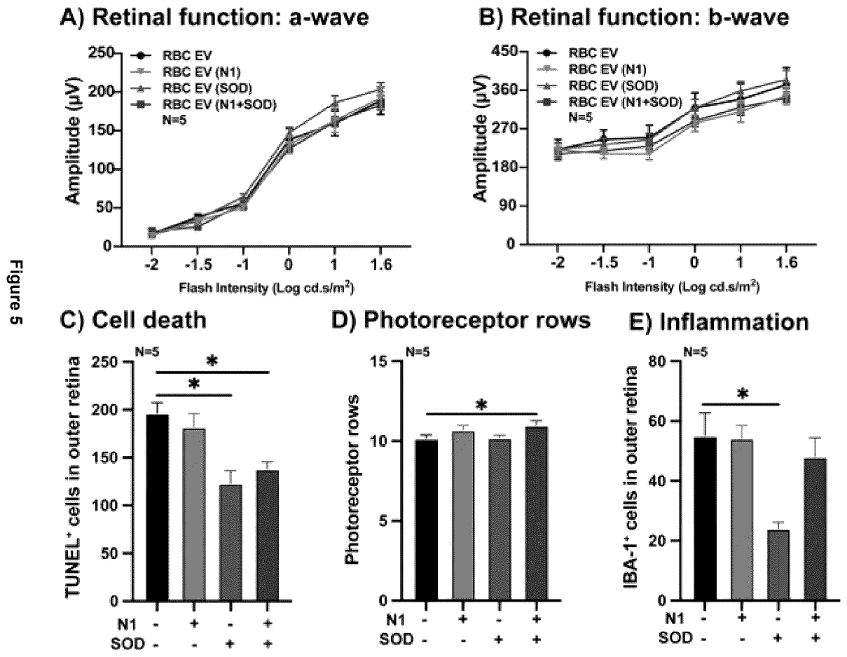

To investigate the potential protective effects of RBC-EV and N1/SOD supplementation on slowing the progression retinal degeneration, RBC-EV (N1/SOD) were injected into mice prior to photo-oxidative damage at a dose of 2.0×10⁹ EV in 1 μl, and compared to RBC-EV, PBS (N1/SOD) and PBS injected controls (FIG. 4A). Retinal function was measured following 5 days of photo-oxidative damage and showed that both RBC-EV (N1/SOD) and PBS (N1/SOD) groups had significantly higher retinal function than no supplement controls, for both a-wave (FIG. 4B, P<0.05) and b-wave (FIG. 4C, P<0.05) measures.

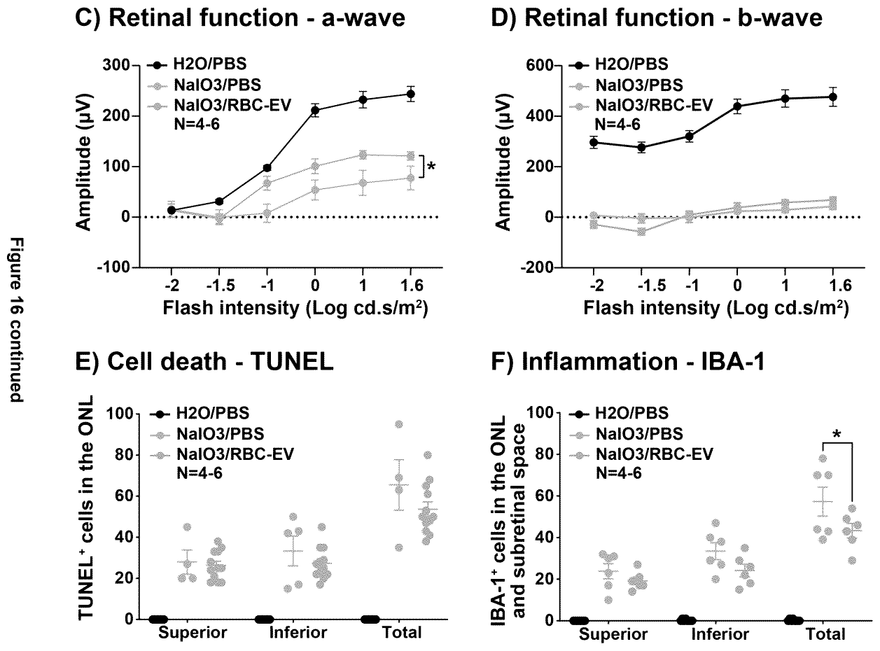

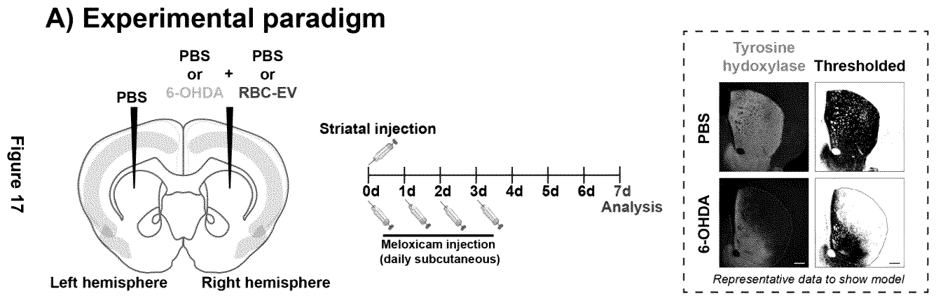

Fundus imaging using MICRON IV show no areas of degeneration in all groups, however vascular bleeds near the optic nerve in PBS (N1/SOD)-injected mice were observed (FIG. 15). Fundus imaging using MICRON IV show no areas of degeneration in all groups, however vascular bleeds near the optic nerve in PBS (N1/SOD)-injected mice were observed (FIG. 15). Photoreceptor cell death was measured using a TUNEL assay and photoreceptor row counts.

The results demonstrated that while both PBS (N1/SOD) and RBC-EV (N1/SOD)-injected mice had significantly reduced numbers of TUNEL+ cells in the ONL compared to no supplement controls (FIG. 4D, P<0.05), RBC-EV (N1/SOD)-injected mice had significantly higher numbers of photoreceptor rows than PBS (N1/SOD) controls (FIG. 4E, P<0.05). Retinal inflammation was further measured using IBA-1 immunohistochemistry, as a marker of microglia/macrophage immune cell presence. RBC-EV (N1/SOD)-injected mice had significantly reduced numbers of IBA-1+ cells in the outer retina compared to PBS controls (FIG. 4F, P<0.05), with no significant differences observed between any other group. Representative confocal images show reduced photoreceptor cell death in supplement groups, with RBC-EV (N1/SOD) showing the lowest level of photoreceptor cell death, and inflammatory cell presence (FIGS. 4G-H).

These results support the use of N1/SOD supplementations in RBC incubation, with the highest retinal protection observed in RBC-EV (N1/SOD) groups compared to no supplement controls.

### Example 5: SOD and N1 and SOD Combination Supplementation Provided Neuroprotective Properties in a Photo-Oxidative Damage-Induced Retinal Neurodegeneration Model

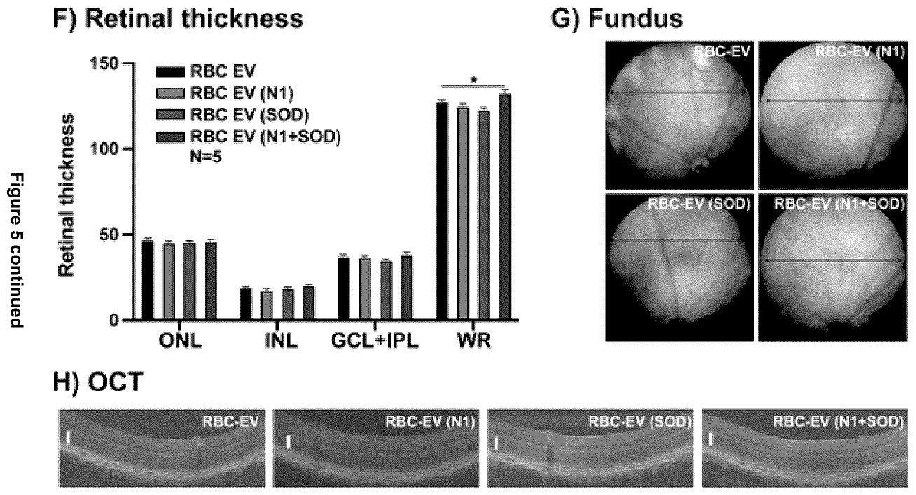

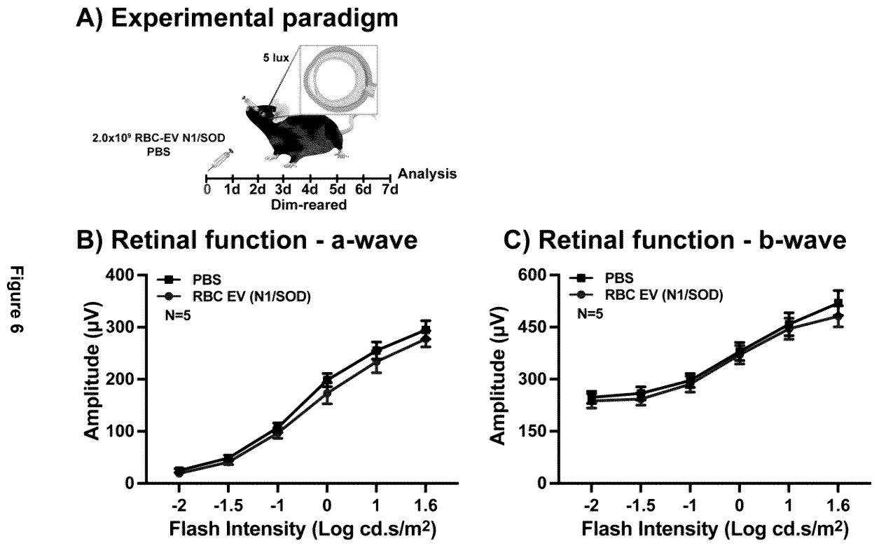

To further investigate the potential protective effects N1 and SOD supplementations on slowing the progression retinal degeneration, RBC-EV (N1), RBC-EV (SOD) and RBC-EV (N1/SOD) were injected into mice prior to photo-oxidative damage at a dose of 2.0×10⁹ EV in 1 μl, and compared to RBC-EV controls. Retinal function was measured following 5 days of photo-oxidative damage and showed no significant difference in a-wave (FIG. 5A, P>0.05) or b-wave (FIG. 5B, P>0.05) measures.

Photoreceptor cell death was measured using a TUNEL assay and photoreceptor row counts. The results demonstrated significantly reduced numbers of TUNEL⁺ cells in the ONL in RBC-EV (SOD) and RBC-EV (N1/SOD)-injected mice (FIG. 5C, P<0.05), and significantly higher numbers of photoreceptor rows in RBC-EV (N1/SOD)-injected mice (FIG. 5D, P<0.05) compared to no supplement controls. Retinal inflammation was further measured using IBA-1 immunohistochemistry, as a marker of microglia/macrophage immune cell presence. While no difference was observed between RBC-EV, RBC-EV (N1) and RBC-EV (N1/SOD)-injected mice, RBC-EV (SOD)-injected mice had significantly reduced numbers of IBA-1⁺ cells in the outer retina (FIG. 5E, P<0.05). OCT thickness measurements showed no change in the ONL, INL, GCL+IPL between any groups (FIG. 5F, P>0.05) but significant increase in whole retinal thickness in RBC-EV (N1/SOD)-injected mice compared to controls. Preserved retinal health was reflected in representative fundus and optical coherence tomography (OCT) images (FIGS. 5G-H).

These results support the use of N1/SOD supplementations in RBC incubation, with reduced cell death compared to no supplement controls.

### Example 6: RBC-EV (N1/SOD) is a Safe Delivery Vehicle

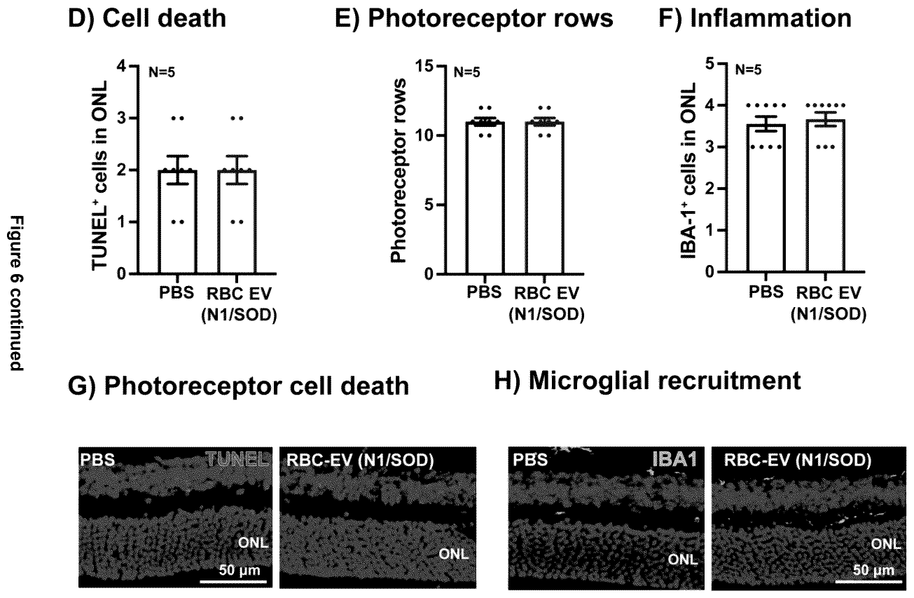

Given the protective effects of N1/SOD supplementation, the safety of RBC-EV (N1/SOD) as a local therapeutic delivery vehicle was assessed. Mice were injected with 2.0×10⁹ RBC-EV (N1/SOD) in 1 μL using intravitreal injection and left for 7 days under standard housing conditions (FIG. 6A). Retinal function was measured using ERG, showing no significant difference in either a-wave (FIG. 6B) and b-wave responses (FIG. 6C) compared to PBS injected controls (P>0.05). No differences were observed for measures of cell death (FIGS. 6D-E; P>0.05), or inflammation (FIG. 6F; P>0.05), as shown in representative confocal images (FIGS. 6G-H). Overall, RBC-EV (N1/SOD) did not confer any toxicity or damage to the retina at the parameters measured, suggesting they can be used as a safe delivery vehicle via local intravitreal injection.

### Example 7: Local Delivery of RBC-EV (N1/SOD) Provides Neuroprotection in a Retinal Neurodegeneration Model

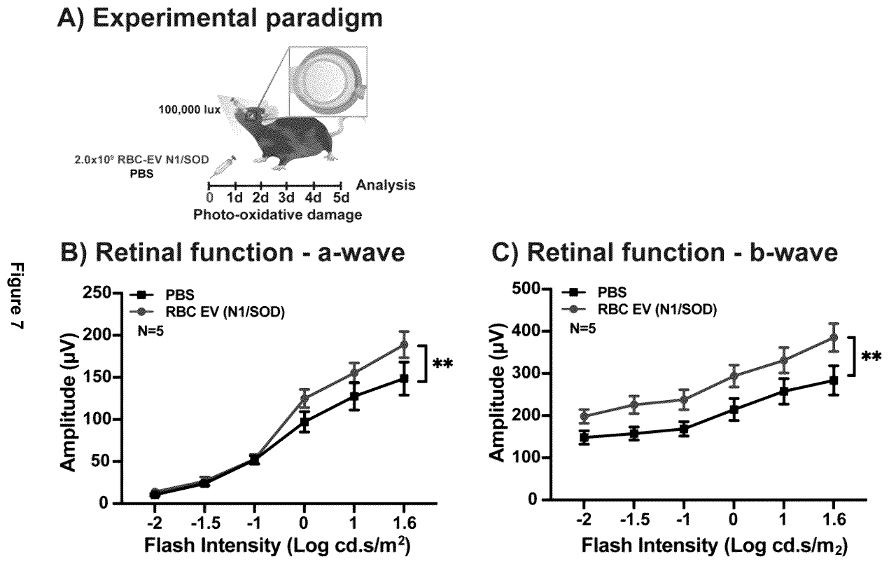

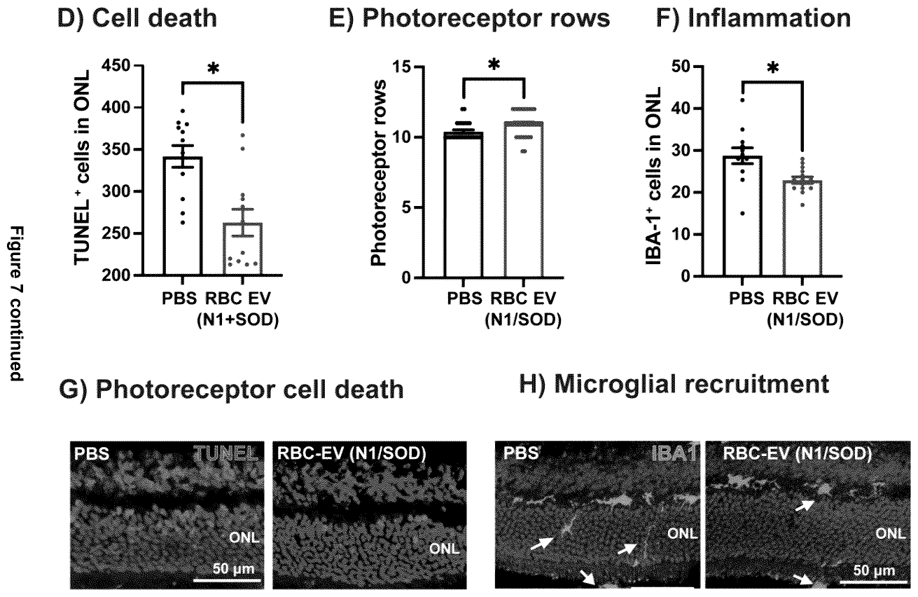

The therapeutic efficacy of RBC-EV (N1/SOD) was investigated in a rodent model of retinal neurodegeneration using intravitreal delivery (FIG. 7A). Following 5 days of photo-oxidative damage, retinal function was measured using ERG.

Results demonstrated that RBC-EV (N1/SOD)-injected mice had significantly preserved function for both a-wave (FIG. 7B, P<0.05) and b-wave (FIG. 7C; P<0.05) responses compared to PBS-injected controls, along with significantly reduced levels of photoreceptor cell death as measured by TUNEL assay (FIG. 7F, P<0.05) and photoreceptor row counts (FIG. 7E, P<0.05). Further, RBC-EV (N1/SOD)-injected mice had significantly reduced numbers of IBA-1+ cells in the outer retina, indicating reduced inflammatory cell presence (FIG. 7F; P<0.05). Representative confocal images demonstrate retinal protection for cell death and inflammatory measures following RBC-EV (N1/SOD) local administration (FIGS. 7G-H).

### Example 8: Systemic Delivery of RBC-EV (N1/SOD) is Safe

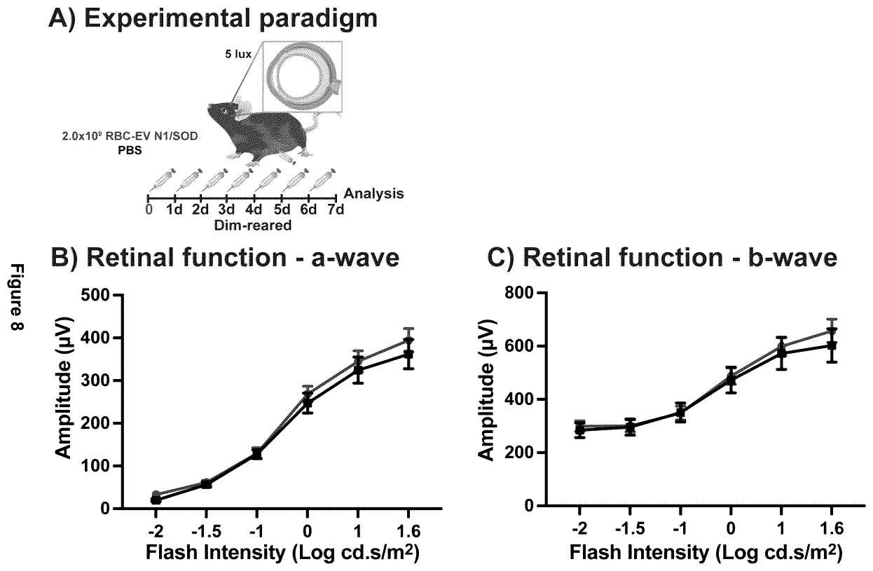

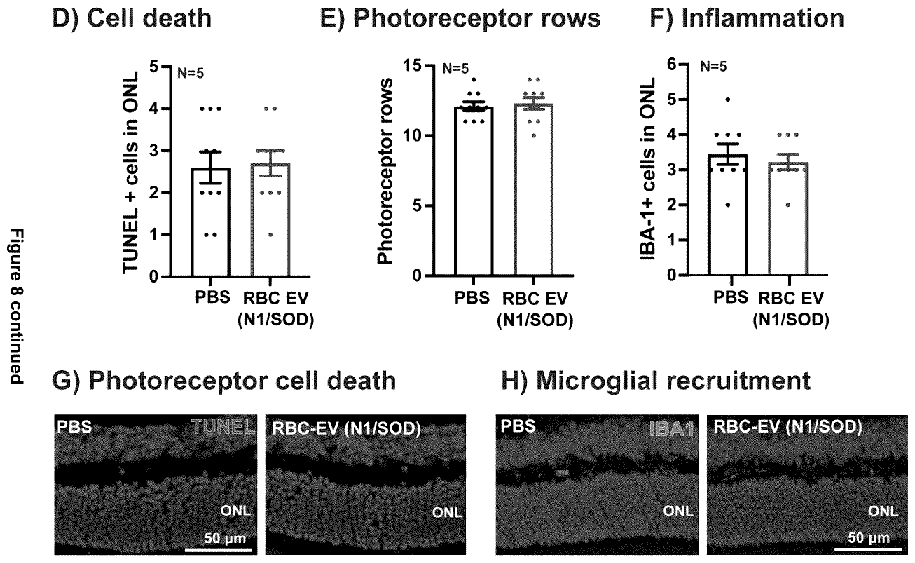

Following the strong safety and therapeutic effects observed in local delivery paradigms, systemic administration of RBC-EV (N1/SOD) was investigated. RBC-EV (N1/SOD) were injected daily for 7 days via intraperitoneal injection at a dose of 2.0×10¹¹ EV in 100 μL and compared to PBS-injected controls (FIG. 8A). As in previous paradigms, retinal functional and morphological assessments were conducted following 7 days of dosing under standard housing conditions. No significant differences were found in retinal function for a-wave (FIG. 8B; P>0.05), or b-wave (FIG. 8C; P<0.05) measures between groups. In addition, there were no differences in levels of cell death (FIGS. 8D-E; P<0.05) or inflammation (FIG. 8F; P<0.05), as shown in representative confocal images (FIGS. 8G-H). Systemic administration of RBC-EV (N1/SOD) did not appear to cause any toxicity to the retina following 7 days of daily dosing.

### Example 9: Systemic Delivery of RBC-EV (N1/SOD) Confers Retinal Protection Against Photo-Oxidative Damage

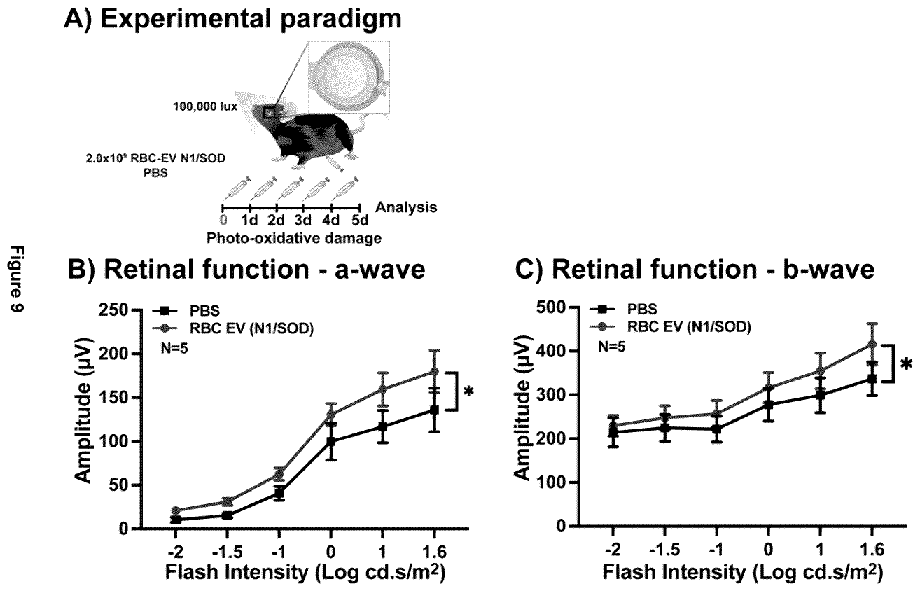

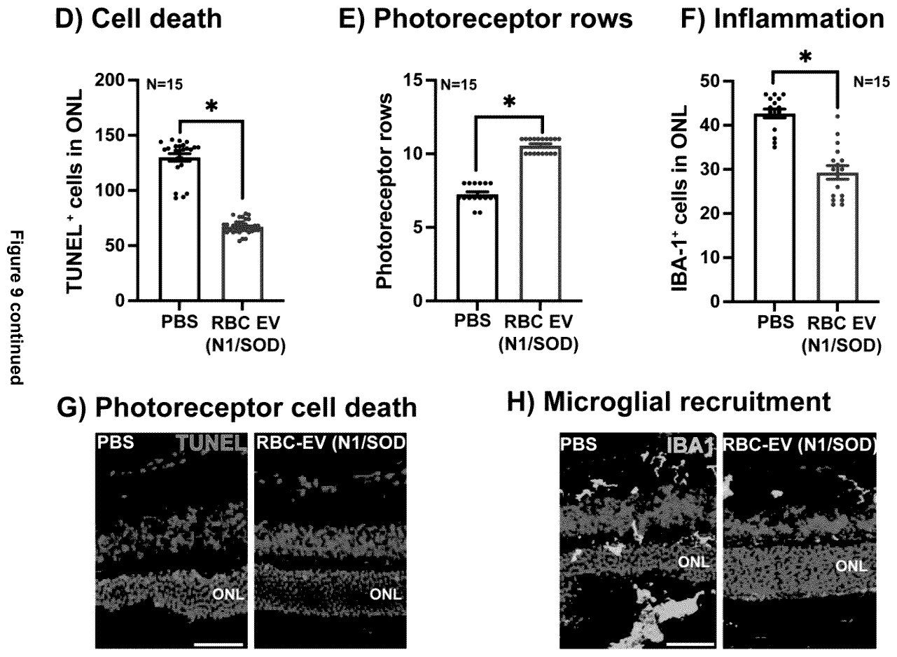

The therapeutic efficacy of systemic RBC-EV (N1/SOD) was investigated by using daily intraperitoneal injections at a dose of 2.0×10¹¹ EV in 100 μL while undergoing 5 days of photo-oxidative damage and compared to PBS (FIG. 9A). Retinal function measured using ERG showed significantly preserved retinal function in RBC-EV (N1/SOD)-injected mice, for both a-wave (FIG. 9B; P<0.05) and b-wave (FIG. 9C; P<0.05) responses. Further, compared to PBS-injected controls, RBC-EV (N1/SOD)-injected mice had significantly reduced levels of TUNEL+ cells in the ONL (FIG. 9D; P>0.0.5), significantly higher numbers of photoreceptor rows (FIG. 9E; P<0.05), and significantly reduced numbers of IBA-1+microglia/macrophages in the outer retina (FIG. 9F; P<0.05). Representative confocal images show decreased levels of cell death and inflammation in the retina of systemically injected RBC-EV (N1/SOD) mice (FIGS. 9G-H), overall suggesting a strong therapeutic protection could be conferred to the retina via systemic administration.

### Example 10: RBC-EV (N1/SOD) Shows Efficient Uptake into Retinal Cells In Vitro and In Vivo

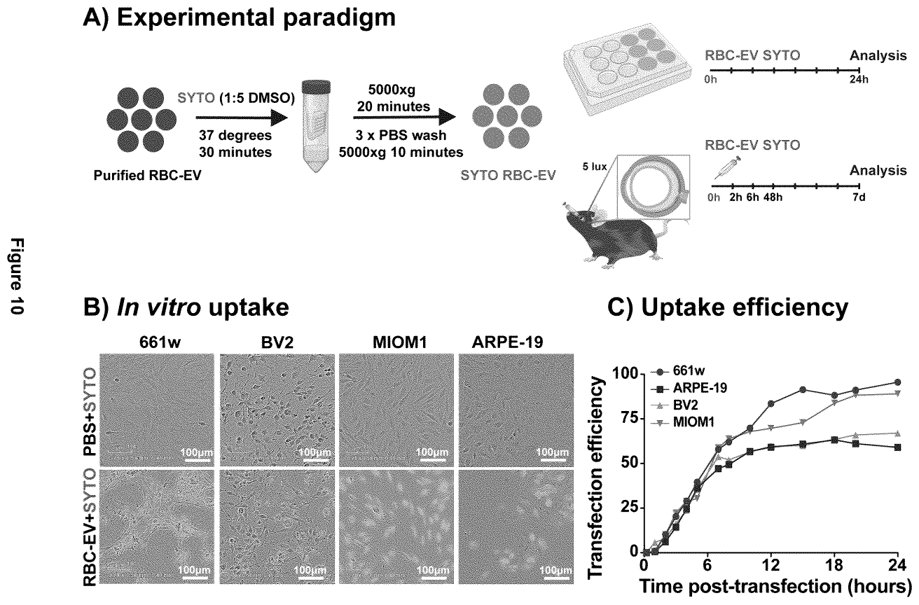

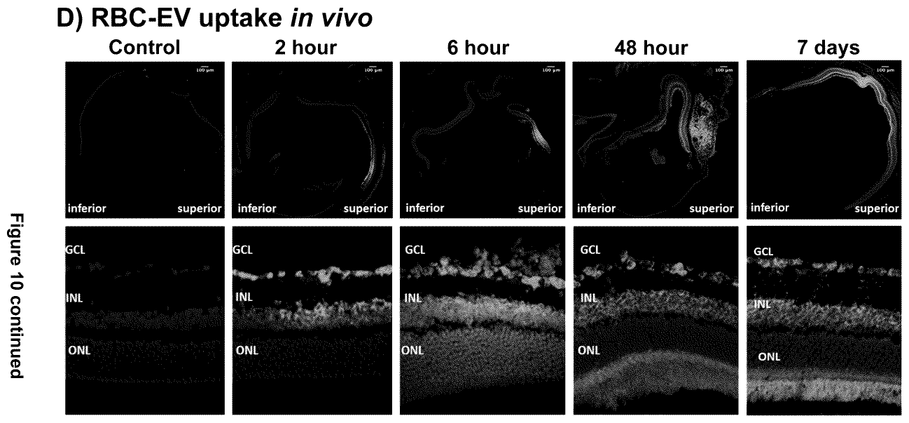

To confirm safety and therapeutic results and determine uptake efficiency of RBC-EV (N1/SOD) in the retina, RBC-EV (N1/SOD) were fluorescently labelled using SYTO RNASelect (green) and incubated on retinal cell lines or injected using intravitreal injection into mice. Uptake efficiency and localisation output measures were observed. (FIG. 10A). In vitro uptake in four retinal cell lines, 661w (photoreceptor cell line), BV2 (microglial cell line), MIOM1 (Muller cell line) and aRPE19 (RPE cell line), was measured over 24 hours using IncuCyte™ ZOOM system. Representative microscope images show fluorescent green labelling in all retinal cell lines following 24 hours incubation (FIG. 10B). Quantification of uptake efficiency shows nearly 100% uptake of RBC-EV (N1/SOD) in both 661w and MIOM1 cell lines by 24 hours, and around 50% uptake in BV2 and aRPE19 cell lines (FIG. 10C). Following, retinal uptake in vivo was observed over 7 days following intravitreal injection at day 0 at a dose of 2.0×10⁹ EV in 1 μl. Uptake could be observed as early as 2 hours post-injection at the site of injection (superior retina) and spread progressively radially across the retina (superior-inferior) as well as through the retinal layers, with strong labelling observed within the ONL by 6 hours and outer segment labelling from 48 hours. Labelling was still observed at 7 days throughout the retina (FIG. 10D).

### Example 11: RBC-EV (N1/SOD) can be Used as miRNA Delivery Vehicles to the Retina

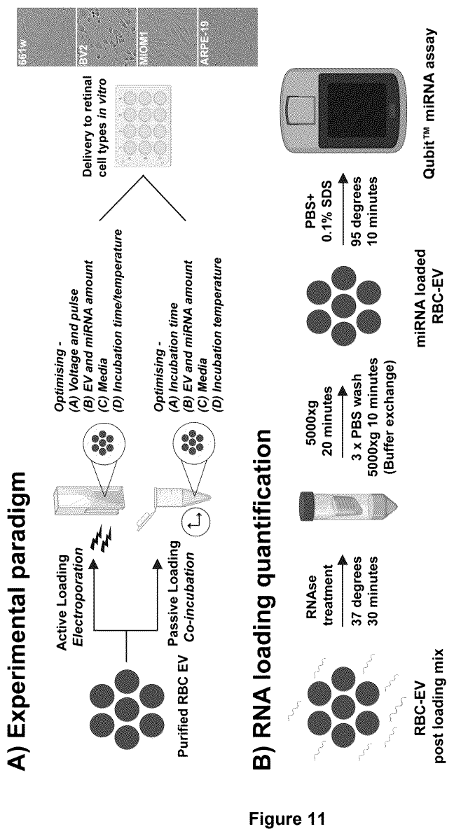

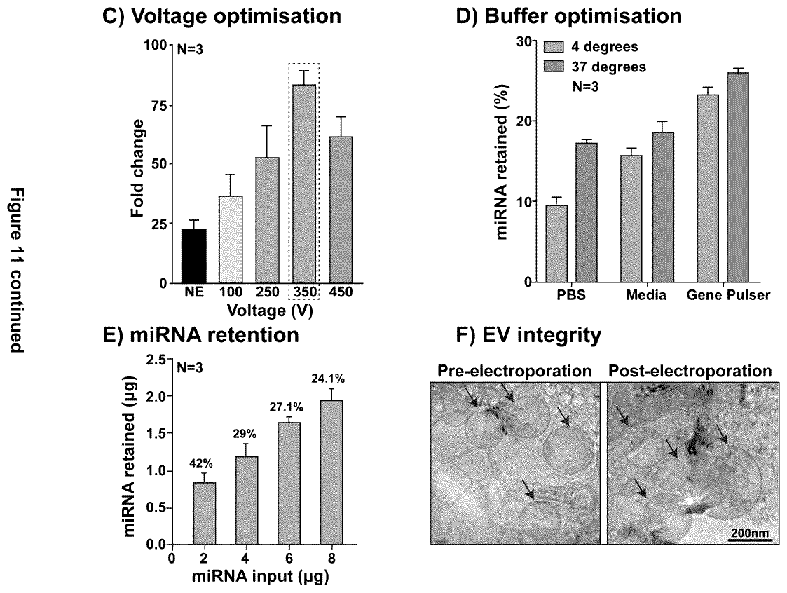

To elucidate if RBC-EV (N1/SOD) can be utilised as therapeutic delivery vehicles, microRNA(miRNA) encapsulation methods were optimised, comparing electroporation and passive loading techniques with modifications to voltage settings, incubation times, temperatures, and buffers (FIG. 11A). Following loading in each optimisation experiment, encapsulated miRNA amounts (μg) were quantified using Qubit miRNA assays (FIG. 11B). miRNA retention was found to increase with increased voltage up to 350 V, with no improvement in miRNA retention found at 450 V (FIG. 11C). Further, it was found that Gene Pulser buffer (BioRad) was the most optimal for miRNA retention, and for all electroporation medias and buffers trialled, the post-electroporation incubation at 37 C improved miRNA retention compared to at 4 °C (FIG. 11D). miRNA retention was not found to increase proportionately to miRNA input amounts, with a lower percent retention as input amount increased (FIG. 11E), suggesting a maximum threshold must exist using electroporation. Finally, RBC-EV (N1/SOD) integrity was assessed pre- and post-electroporation at optimal settings (350 V, Gene Pulser buffer, 37 C post-incubation) using cryo-EM. No obvious morphological changes or aggregation were found in RBC-EV (N1/SOD) following electroporation (FIG. 11F).

Taken together these results support the capacity for RBC-EV (N1/SOD) to be used in therapeutic miRNA encapsulation for delivery to the retina.

### Example 12: N1 and SOD Supplementation Confers Protection to 661w-Photoreceptor Cells In Vitro and Enhances EV Production

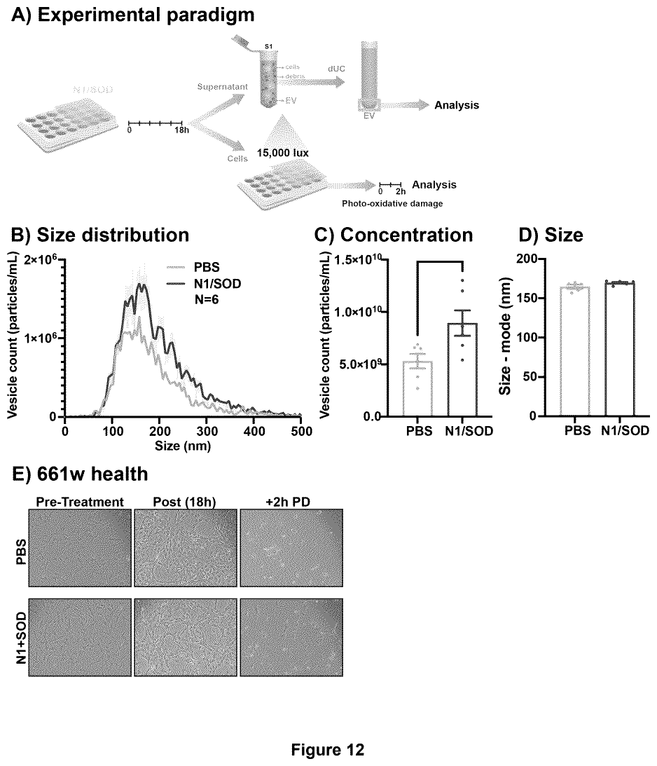

To verify the protective properties of N1 and SOD supplementation on cell health, retinal health and EV health, 661w photoreceptor-like cells were incubated in the presence or absence of N1/SOD for 18 hours prior to 2 hours photo-oxidative damage at 15,000 lux (FIG. 12A). EV distribution profile and size determined from Nanoparticle tracking analysis shows a significant increase in EV numbers but not size following N1/SOD supplementation (FIG. 12B-D, P<0.05). Representative images taken prior to N1/SOD administration, after 18 hours incubation, and following 2 hours photo-oxidative damage shows that while there was no change in visible cell morphology and confluency measures between control and supplementation groups at 0 and 18 hours, following photo-oxidative damage, cells which had been incubated with N1/SOD had better preserved morphology (processes) and less visible cell death (rounded and dark cells) than 661w control cells (FIG. 12E).

Taken together these results suggest that 661w incubated with N1/SOD supplementations had increased protection against photo-oxidative damage and an increased level of EV production. (N=3).

### Example 13: RBC Antioxidant Capacity is Increased with N1/SOD Supplementation

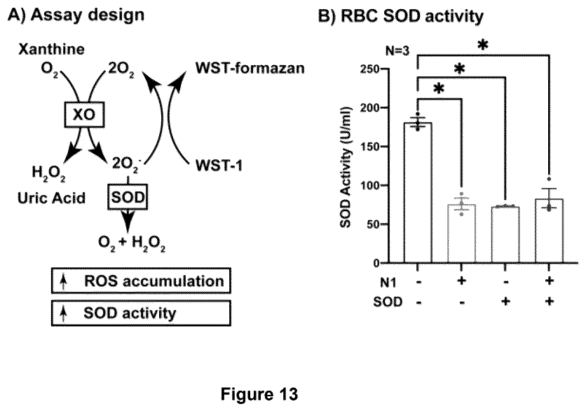

The level of SOD activity as measured by superoxide quenching capacity was measured in RBC with and without N1 or SOD supplementation using a SOD colorimetric activity assay. Increased SOD activity was correlated to increased reactive oxygen species (ROS) accumulation (FIG. 13A). The results demonstrated that compared to controls, RBC (N1), RBC (SOD) and RBC (N1/SOD) had significantly reduced SOD activity, supporting a reduced build-up of ROS inside cells post 18 h incubation with supplementations (FIG. 13B, P<0.05). No significant difference was seen in the SOD activity between supplementation groups (FIG. 13B, P>0.05). This result suggests that N1, SOD or N1/SOD supplementations can improve the antioxidant capacity of RBC.

### Example 14: RBC Health and Quality Assessments Following N1/SOD Incubation

RBC were assessed for morphological changes using ImageJ plug-in (FIG. 14A) before culture and (FIG. 14B) following incubation at 37 °C overnight with N1/SOD supplementation (FIG. 14C-F). The number of abnormalities (tear drop shape, variation in size/shape and presence of burr cells) was counted and calculated as a percentage against normal RBC.

### Example 15: Systemic Delivery of RBC-EV (N1/SOD) Protects Against Sodium-Iodate-Induced Pan Retinal Neurodegeneration

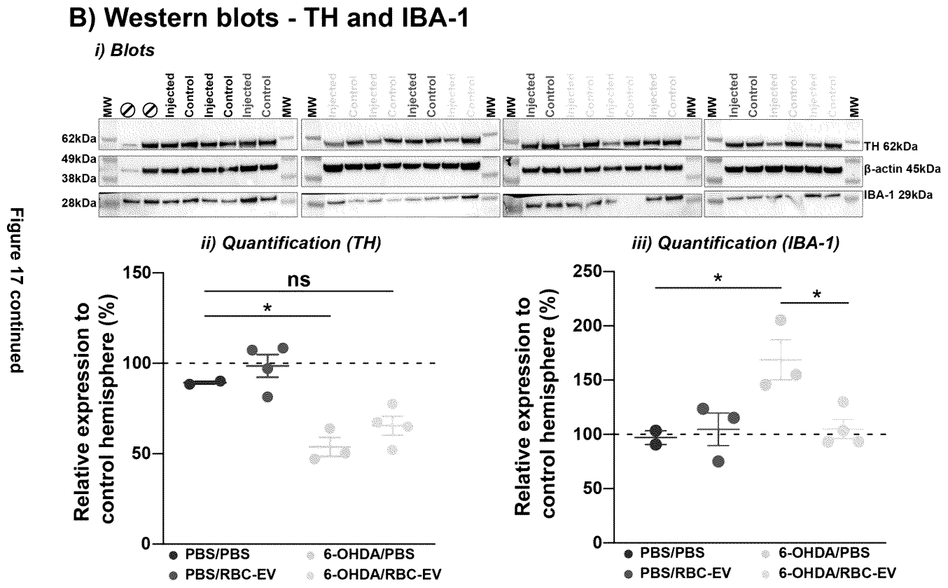

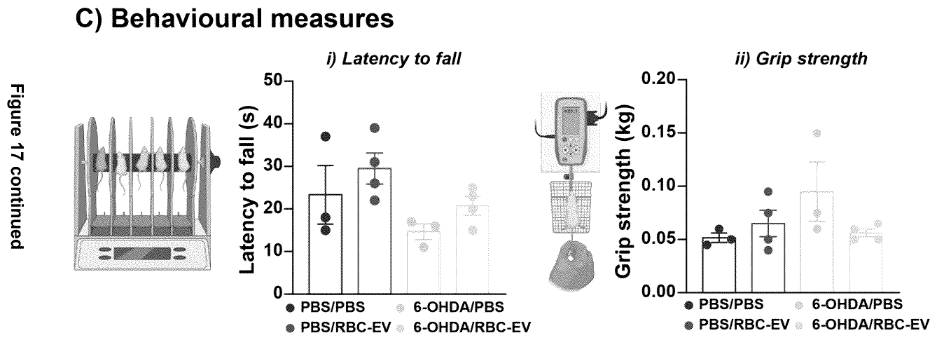

Given the observed local and systemic protection of RBC-EV (N1/SOD) administration against photo-oxidative damage-induced retinal degeneration, the therapeutic efficacy of RBC-EV (N1/SOD) was explored in a secondary model of retinal degeneration-Sodium Iodate (NaIO₃) (FIG. 16A). Compared to PBS-injected controls, RBC-EV (N1/SOD)-injected mice had improved retinal function for a-wave measures (FIG. 16B-C; P<0.05). While no significant difference was observed between groups for b-wave responses (FIG. 16D, P>0.05), this reflects a feature of the chemical nature of the model and was not expected to be improved regardless of treatment. Photoreceptor cell death (TUNEL) and immune cell presence as a marker of inflammation (IBA-1) were also measured showing that there was a non-significant reduction in cell death in RBC-EV (N1/SOD) treated mice (FIG. 16E, P>0.05), and a significant reduction in total IBA-1+ immune cells compared to PBS-injected controls (FIG. 16F, P<0.05). Mice injected with H₂O were also included as baseline controls with comparisons demonstrating that NaIO₃-injected mice treated with RBC-EV (N1/SOD) were closer to control levels for all measures compared to non-treated mice (FIG. 16A-F). Overall, these results show significant improvement in retinal function and inflammation in mice treated with RBC-EV (N1/SOD) against NaIO₃-induced retinal degeneration, further supporting the therapeutic potential of this treatment.

### Example 16: RBC-EV (N1/SOD) is Safe and Provides Therapeutic Protection Against Neuro-Inflammation and Motor Deficit Features in Parkinson's Disease Model of Neurodegeneration

To test the broad applicability of RBC-EV (N1/SOD) treatment, RBC-EV (N1/SOD) were administered locally (striatal injection) in a neurotoxin model of Parkinson's disease (6-OHDA). The 6-OHDA model of Parkinson's disease causes the selective destruction of dopaminergic and noradrenergic neurons through generation of reactive oxygen species (Masini et al., 2021). Due to the structural similarities of 6-OHDA to dopamine, dopaminergic cells uptake 6-OHDA via dopaminergic receptors and membrane transporters and this accumulates in these neurons. The 6-OHDA is subsequently oxidised by the enzyme monoamine oxidase resulting in the release of reactive catecholamine quinones, hydrogen peroxide, and other reactive oxygen species. When injected directly into catecholaminergic regions such as the striatum, 6-OHDA rapidly depletes dopaminergic neurons which progressively causes dopaminergic denervation over the course of 3-5 weeks which can be observed through behavioural testing, molecular, and histochemical analysis.

Neuro-motor responses as well as measures of neuro-inflammation were assessed 7 days post degeneration-induction/treatment-administration in vehicle and RBC-EV (N1/SOD)-injected control (healthy; no 6-OHDA) and degenerative mice (6-OHDA) (FIG. 17A). Western blot was performed against Tyrosine Hydroxylase (TH) and IBA-1 as respective measures of dopamine production in dopaminergic neurons (neuronal activity), and immune cell presence (inflammation) (FIG. 17B(i)). To test neuromotor deficits as a result of 6-OHDA, the forelimb strength of mice was tested using a grip-strength paradigm.

Mice injected with PBS/6-OHDA were found to have significantly reduced levels of TH compared to PBS-injected controls (FIG. 17B(ii), P<. 0.05) supporting the use of 6-OHDA to induce dopamine loss. No significant differences were observed between control or degenerative groups (FIG. 17B(ii), P>0.05), indicating that while RBC-EV (N1/SOD) was safe it did not provide strong therapeutic protection against TH loss at this early disease time-point investigated. Importantly, IBA-1 levels were found to be significantly increased in 6-OHDA/PBS-injected mice compared to PBS/PBS-injected controls, as well as in comparison to 6-OHDA/RBC-EV (N1/SOD)-injected mice (FIG. 17B(iii), P<0.05). No difference was measured in IBA-1 levels in 6-OHDA/RBC-EV (N1/SOD)-injected mice compared to controls indicating both the safety and strong therapeutic efficacy of RBC-EV (N1/SOD) treatment in reducing neuroinflammation.

Finally, while no significant differences were observed in either rotarod or grip strength behavioural measures between any group, mice treated with 6-OHDA/RBC-EV (N1/SOD) had increased latency to fall compared to 6-OHDA/PBS-injected mice (FIG. 17C(i), P>0.05), and should be further evaluated for potential improvements at a later time point when behavioural effects are more apparent in this model. Overall, these results support the use of RBC-EV (N1/SOD) as a safe and efficacious therapeutic against early neuroinflammatory features of Parkinson's disease with further evaluation required at later time points in the model to see efficacy against neuromotor deficits.

### Example 17: RBC-EV (N1/SOD) Outperform Commercial Competitor RBC-EV Products in Protecting Against Retinal Degeneration In Vivo

RBC-EV (N1/SOD) (RBC-EV CVR) were compared against RBC-EV from a market competitor (RBC-EV Comp) along with respective vehicle controls. PBS-injected mice, healthy mice (dim-reared; DR), and un-injected mice (photo-oxidative damage; PD) were also included as control groups.

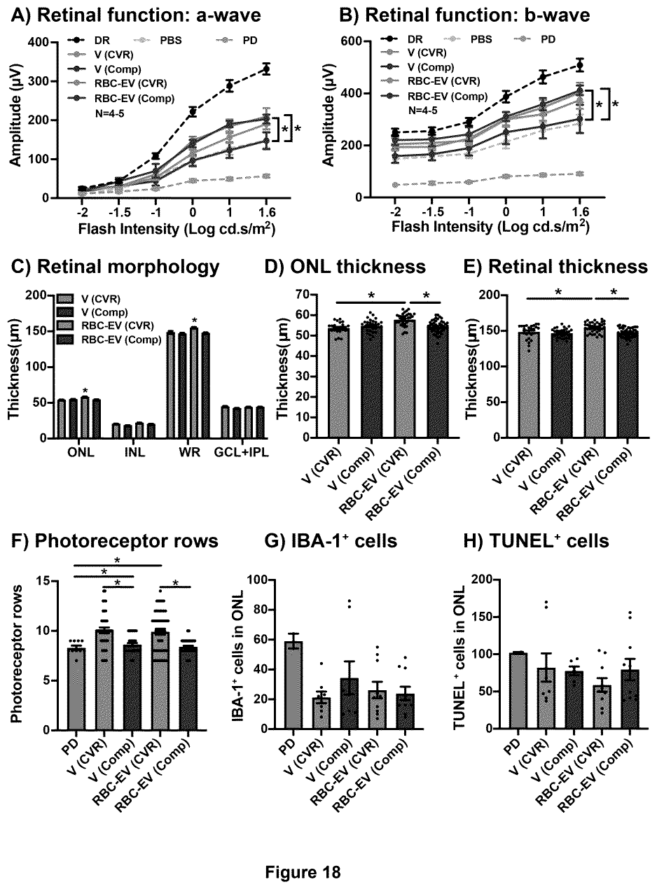

Functional assessment showed that following 5 days photo-oxidative damaged mice injected with RBC-EV (Comp) or Vehicle (CVR) had significantly preserved retinal function compared to PBS-injected controls for both a-wave (FIG. 18A, P<0.05) and b-wave (FIG. 18B, P<0.05) measures. No significant difference in function was observed between RBC-EV (CVR) or RBC-EV (Comp), or between RBC-EV (CVR) and Vehicle (CVR) groups, however RBC-EV (Comp) and Vehicle (CVR)-injected mice had significantly preserved retinal function compared to Vehicle (Comp). No significant difference was observed between PBS-injected controls and Vehicle (Comp) suggesting this vehicle has no protective effects to the retina, while Vehicle (CVR) was found to be protective against retinal degeneration (FIGS. 18A and B).

Retinal thickness measures show that mice injected with RBC-EV (CVR) had a significantly thicker outer nuclear layer (ONL) and whole retinal thickness compared to RBC-EV (Comp) and Vehicle (CVR) groups (FIG. 18C-E), indicating increased survival of photoreceptors in these mice. Increased photoreceptor survival can also be shown by increased photoreceptor row counts in both Vehicle (CVR) and RBC-EV (CVR) groups compared to PD controls, Vehicle (Comp), and RBC-EV (Comp) groups, respectively (FIG. 18F, P<0.05).

No significant differences in inflammation (IBA-1⁺ cells in the outer retina) or cell death (as measured by TUNEL⁺ cells in the ONL) were shown between any groups (FIGS. 18G and H, P>0.05). These results support the protective effects of RBC-EV (N1/SOD) and overall show stronger therapeutic efficacy in protecting against retinal degeneration over lead commercial RBC-EV (Comp) product tested.

### Example 18: Antioxidant Supplementation Testing on RBC/RBC-EVs

Resveratrol is a naturally occurring phytoestrogen polyphenol/stilbene found in red grapes, berries, and peanuts with high antioxidant properties. Resveratrol acts to induce antioxidant enzymes such as SOD, glutathione peroxidase-1, heme-oxygenase and inhibit ROS production. Resveratrol has demonstrated protective effects in cancer, cardiovascular disease, and neurodegenerations, with anti-apoptotic, anti-angiogenic, and anti-inflammatory effects (Salehi et al., 2018).

Kaempferol is a phytoestrogen polyphenol flavonol/flavonoid natural antioxidant found in fruits and vegetables with known antioxidant and anti-inflammatory effects. Kaempferol acts through metalloproteinase inhibition, ROS inhibition, and SOD and glutathione modulation (Silva, 2012).

Glutathione is a powerful non-protein/non-enzymatic thiol antioxidant and one of the most important in the body, produced by cells in the cytosol (not dietary). Glutathione is present in millimolar concentrations. Glutathione exists in a reduced (GSH) and oxidised (GSSG) state, with the ratio of GSH:GSSG indicative of cellular stress (higher GSSG=more oxidative stress). Glutathione protects cells by scavenging/reducing ROS and as a cofactor for detoxification enzymes such as glutathione peroxidases (Averill-Bates, 2023). In the retina, GSH is the most powerful and abundant antioxidant, and is reduced in patients with retinal degeneration (Sreekumar et al., 2021).

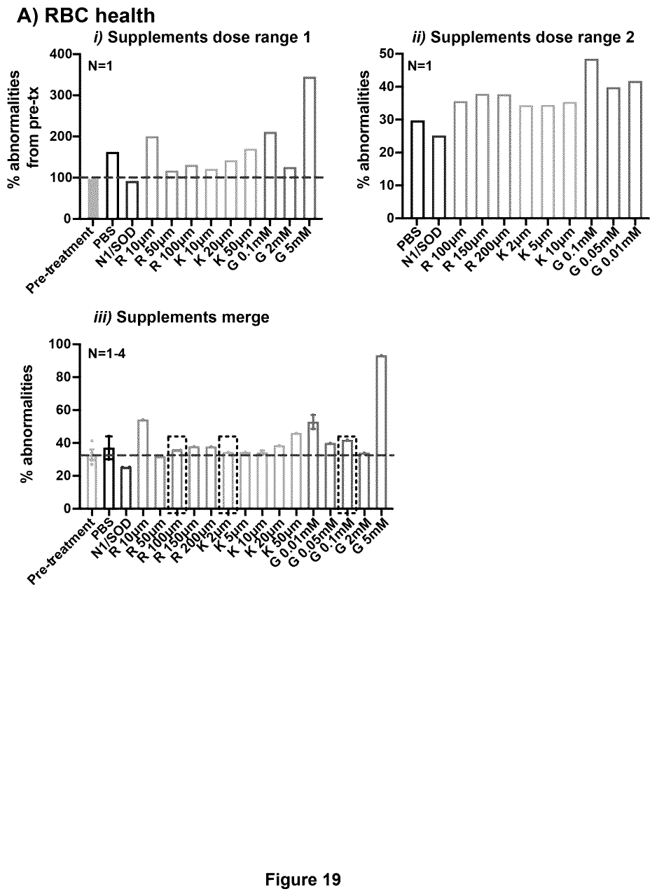

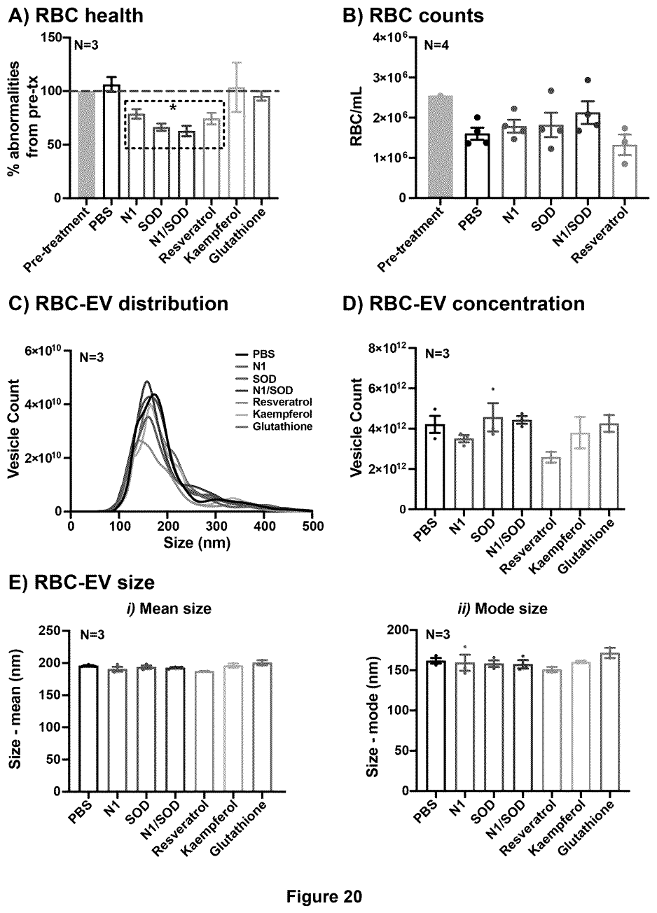

Compared to pre-treatment, RBC treated with N1/SOD had the lowest % membrane abnormalities compared to PBS and all doses of RBC treated with resveratrol, kaempferol and glutathione (FIG. 19A). Out of the dose ranges, resveratrol (R 100 μM), kaempferol (K 2 μM) and glutathione (G 0.1 mM) were found to produce RBC with the lowest % membrane abnormalities compared to other doses tested (FIG. 19A). These doses were chosen for future testing and were compared to RBC (PBS), RBC (N1), RBC (SOD) and RBC (N1/SOD) groups. Results demonstrated that compared to pre-treatment RBC, RBC (N1), RBC (SOD), RBC (N1/SOD) and RBC (R 100 μM) had significantly reduced % membrane abnormalities (FIG. 20A, P<0.05), while RBC (PBS), RBC (K 2 μM) and RBC (G 0.1 mM) had no observed protection (FIG. 20A, P>0.05). Of all supplements tested, RBC (N1/SOD) had the lowest % membrane abnormalities. RBC counts were also performed to assess RBC loss compared to freshly isolated RBC (pre-treatment), with the highest preservation of RBC counts in RBC (N1/SOD) group and lowest in RBC (PBS) and RBC (R 100 μM) groups. (FIG. 20B) RBC-EV size distribution was also compared between supplement groups, with the most uniform distribution observed in RBC-EV (N1), RBC-EV (SOD), and RBC-EV (N1/SOD) groups, while RBC-EV (PBS), RBC-EV (R 100 μM), RBC-EV (K 2 μM) and RBC-EV (G 0.1 mM) distribution profiles were heterogeneous/non-uniform and skewed (FIG. 20C). Results showed a reduction in RBC-EV concentration for RBC-EV (N1) and RBC-EV (R 100 μM) compared to other groups, however this was not significant (FIG. 20D, P>0.05). No differences in mean (FIG. 20E) or mode (FIG. 20F) size was observed between any supplement groups (P>0.05). Overall, RBC-EV (K 2 μM) and RBC-EV (G 0.1 mM) showed the least protective effects on both RBC and RBC-EV quality and quantity.

### Example 19: Effects of Supplementation on Endogenous Properties of RBC and RBC-EV

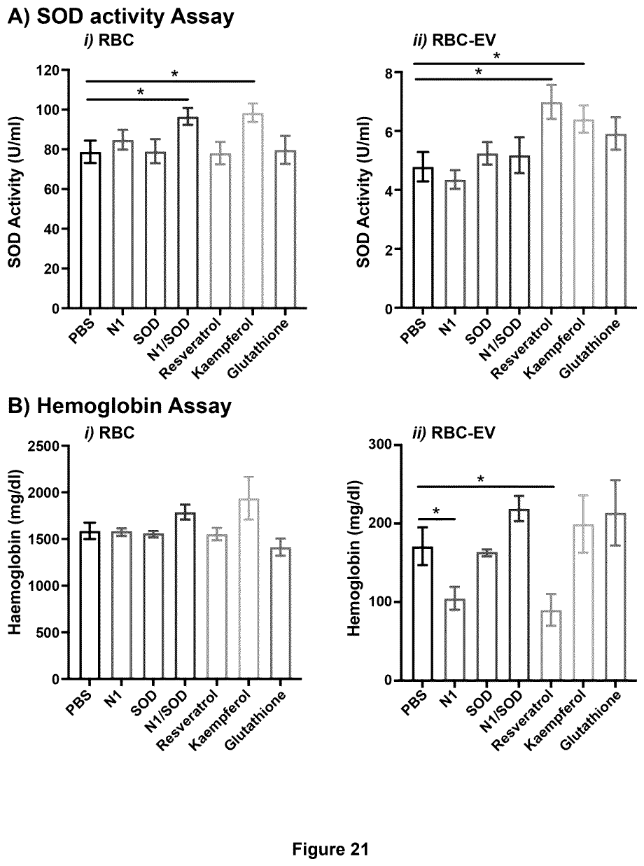

To assess the endogenous levels of SOD and haemoglobin in RBC and RBC-EV, in response to supplementations, SOD activity assay and haemoglobin assays were performed on RBC and RBC-EV following 18 h incubation. Results demonstrated that RBC incubated with (N1/SOD) and (K 2 μM) had significantly higher SOD activity compared to RBC (PBS) controls (FIG. 21A(i), P<0.05), while RBC-EV (R 100 μM) and RBC-EV (K 2 μM) had significantly increased SOD activity than RBC-EV (PBS) controls (FIG. 21A(ii), P<0.05). No significant difference in haemoglobin levels was measured between any RBC groups (FIG. 21B(i), P>0.05), however RBC-EV (N1) and RBC-EV (R 100 μM) had significantly reduced levels of haemoglobin compared to RBC-EV (PBS) controls (FIG. 21B(ii), P<0.05).

### Example 20: Assessing In Vivo Protection from RBC-EV Resveratrol Supplementation

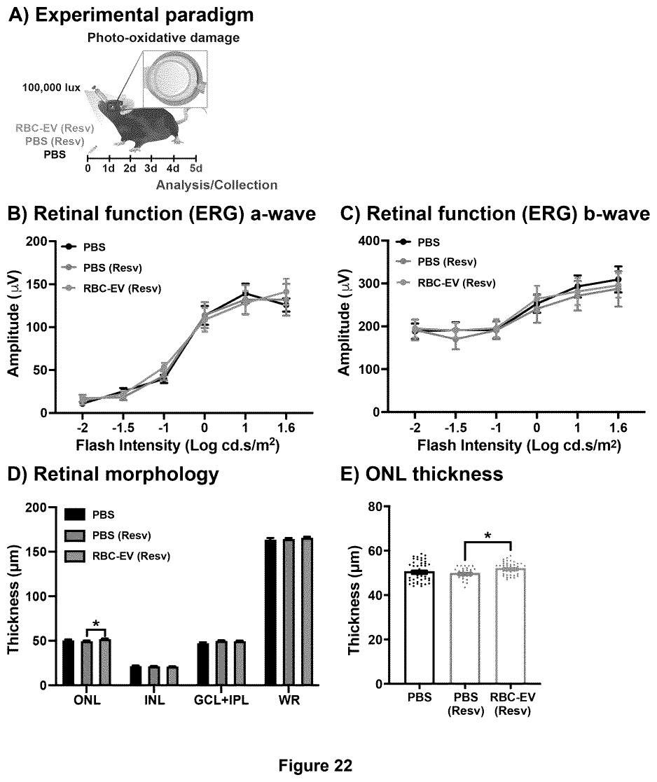

Following in vitro testing and analyses, RBC health was found to be significantly preserved (reduced % membrane abnormalities) compared to pre-treatment controls when incubated with resveratrol (100 μM). Therefore, RBC-EV (Resv) were tested for their therapeutic efficacy in vivo in a photo-oxidative damage model of retinal degeneration (1), and compared to PBS (Resv 100 μM) and PBS controls (FIG. 22A).

Following 5 days of photo-oxidative damage retinal function and morphology were measured. No significant difference was observed in retinal function for a-wave (FIG. 22B, P>0.05), or b-wave (FIG. 22C, P>0.05), measures. Retinal thickness measures were performed on OCT images, with a significant increase in outer nuclear layer (ONL) thickness between PBS (Resv) and RBC-EV (Resv) groups (FIG. 22D-E, P<0.05), however no significant differences were seen in any other groups or retinal layers measured. Overall, RBC-EV (Resv) did not confer significant protection to the retina against photo-oxidative damage-induced retinal degeneration, although it did increase ONL thickness.

### Example 21: Human RBC and RBC-EV Characterisation

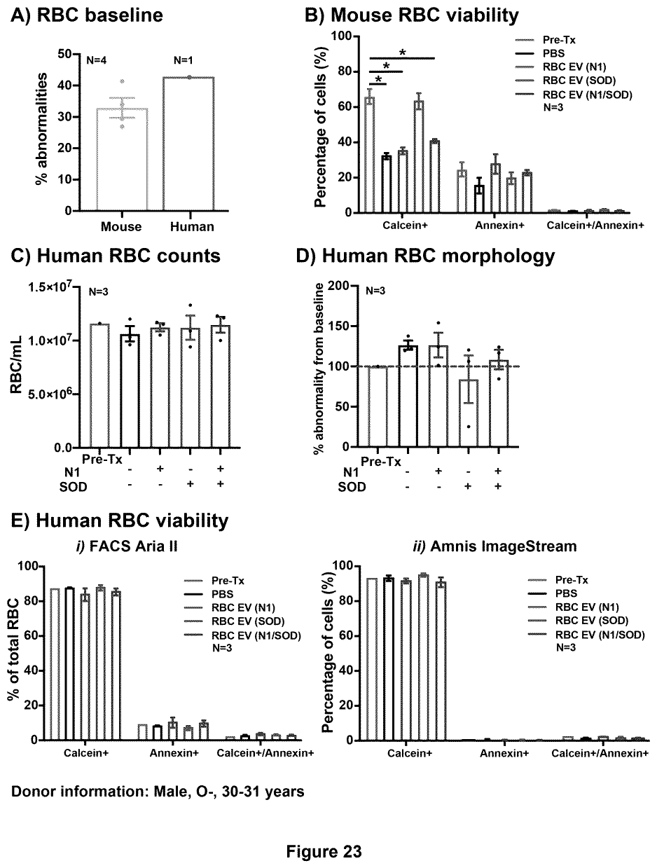

To validate our RBC incubation pipeline in mice, human blood was collected from LifeBlood Australia and incubated in 1:10 PBS with N1, SOD or N1/SOD supplementations as per the above methodology. Baseline abnormality measures were taken prior to incubation and compared to mouse RBC % abnormalities. Mouse RBC showed ~30% abnormalities (N=4) while human RBC had ~40% (N=1) at baseline (FIG. 23A). Mouse RBC viability was analysed by the % of Calcein⁺ (viability marker), Annexin V⁺ (apoptosis marker) and double⁺ cells, using imaging flow cytometry (Amnis, ImageStream) analysis. Results showed a significant decrease in the % of Calcein⁺ RBC in RBC (PBS), RBC (N1) and RBC (N1/SOD) groups compared to pre-treatment controls (FIG. 23B, P<0.05). No differences were observed between groups for % of Annexin⁺ or double⁺ cells (FIG. 23B, P>0.05).

Human RBC numbers were counted following 18 h incubation in supplementations and compared to pre-treatment RBC, with no significant differences in numbers observed between groups (FIG. 23C, P>0.05). Human RBC health was assessed, with the lowest % abnormalities seen in RBC (SOD) and RBC (N1/SOD) groups (FIG. 23D). Human RBC viability was analysed using flow cytometry as was done for the mouse RBC. Results for both FACS Aria II and Amnis ImageStream analyses showed high RBC viability across all RBC groups, with no significant differences between groups (FIG. 23E, P>0.05).

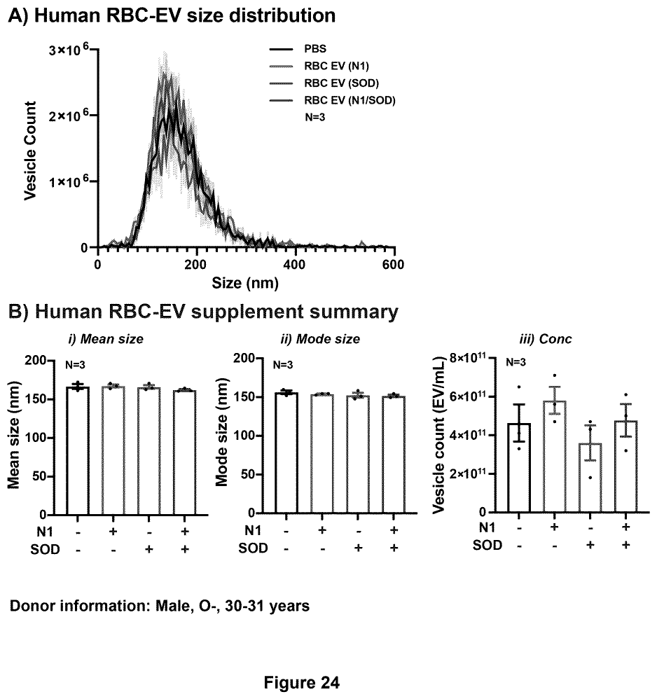

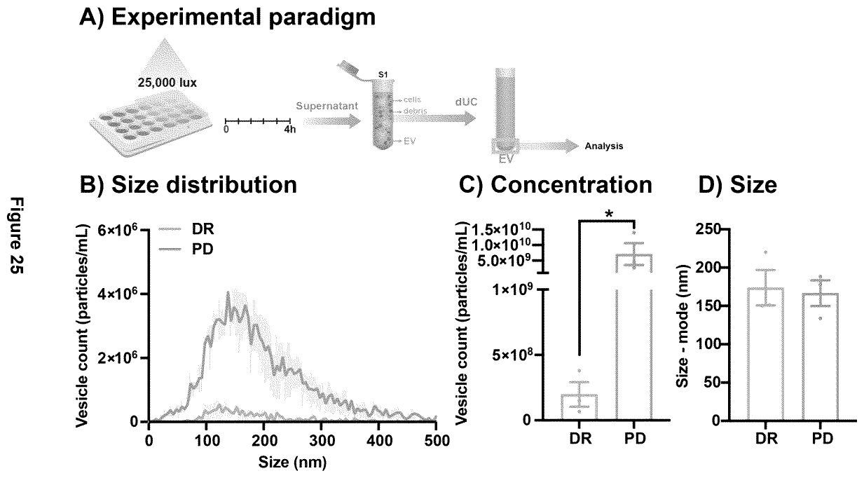

Collectively these results indicate that human RBC were of high quality, with SOD and N1/SOD supplementations showing the strongest preservation of RBC health. RBC-EV characterisation and quality was also performed on human RBC groups, showing no significant difference in size distribution profile or mean or mode size (FIG. 24A-B, P>0.05), and no significant difference in concentration between groups (FIG. 24B(i)-(iii), P>0.05). Human RBC-EV distribution and mean and mode size for supplement groups were highly comparable to mouse blood indicating a robust and transferable pipeline.

### Example 22: In Vitro Photo-Oxidative Damage Induces Photoreceptor-EV Release

To assess the effects of photo-oxidative damage on photoreceptors, 661w-photoreceptor-like cells were subjected to 25,000 lux bright white light for 4 hours and EV were collected from the cell supernatant and compared to dim controls (standard cell culture incubator environment; DR) (FIG. 25A). EV distribution profile and size determined from Nanoparticle tracking analysis shows a significantly increased number of 661w-EV following photo-oxidative exposure compared to control cells (FIG. 25B-C, P<0.05), however no change was seen in their mode size (FIG. 25D, P>0.05). Overall, these results support an increase in photoreceptor-EV release following stress.

### Example 23: Effect of Supplementations on 661w Health and Protection Against Photo-Oxidative Damage

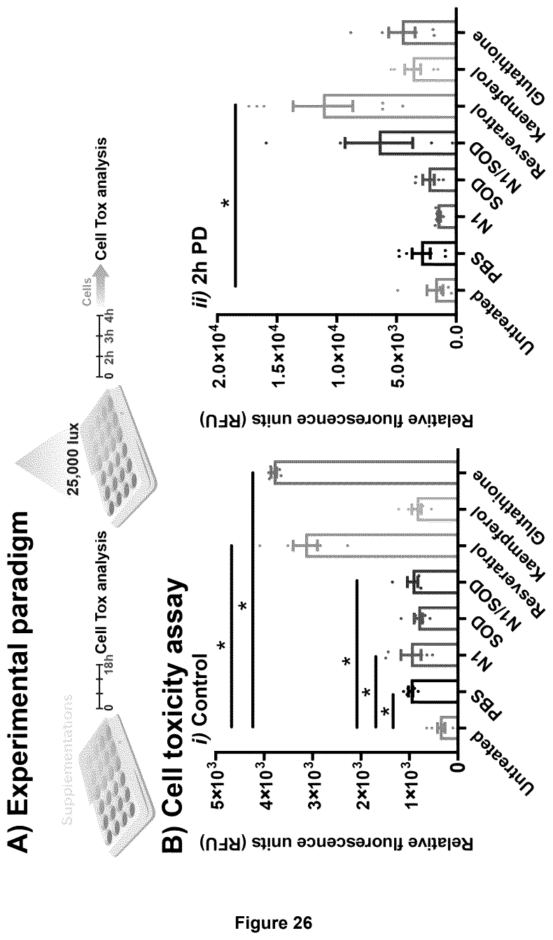

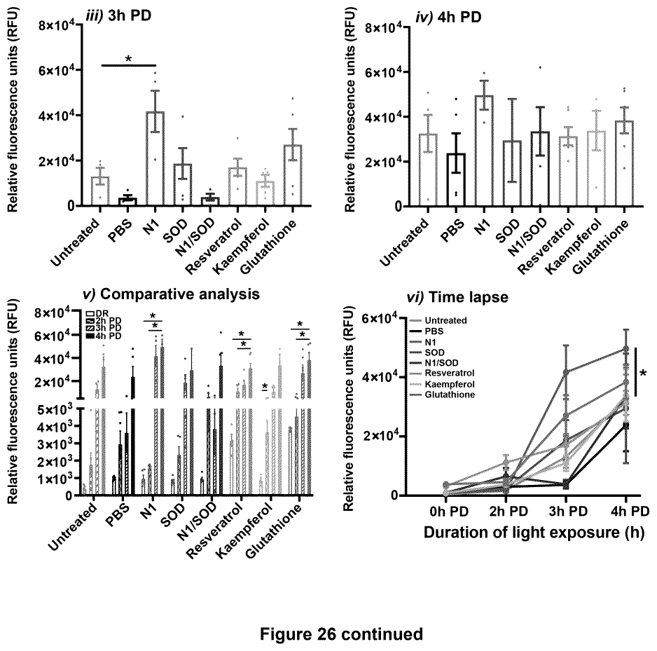

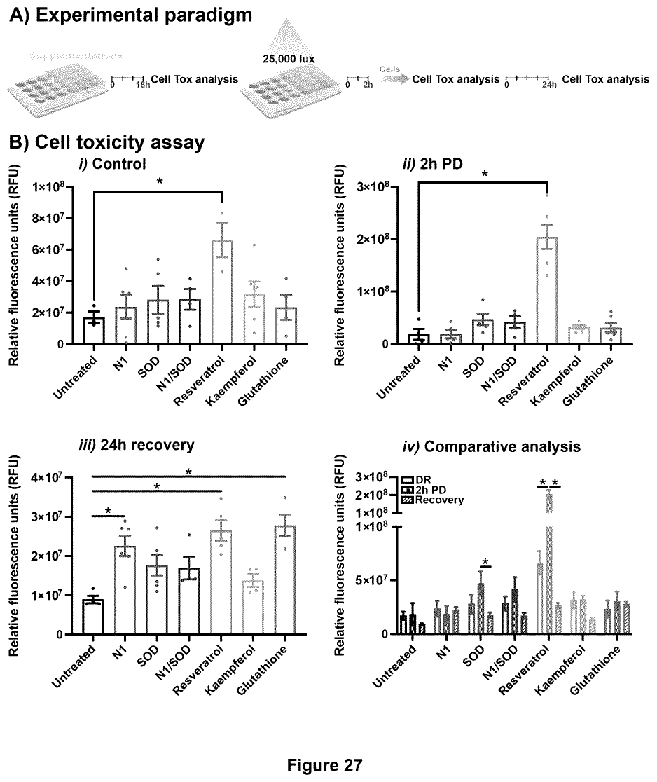

To quantify the protective capabilities of supplementation on 661w cells in response to photo-oxidative damage, N1 and SOD were compared individually, and in combination, compared to Resveratrol, Kaempferol and Glutathione supplementations and PBS and untreated controls. Supplementations were incubated on 661w for 18 hours prior to photo-oxidative damage, which was carried out at 25,000 lux bright white light for 2, 3, and 4 hours, as well as 2 hours followed by 24 hours recovery in standard conditions. Supplementations were used at either full dose (R 100 μM, K 2 μM and G 0.01 mM) (FIG. 26A) or half doses (R 50 μM, K 1 μM and G 0.005 mM) (FIG. 27A). Cell health was assessed using a cell toxicity assay.

Results indicated that in baseline (0 h) groups, compared to untreated cells some toxicity was caused by supplementations, except for SOD and K 2 μM. Following 2 and 3 hours of PD however, R 100 μM and N1 supplementations were found to confer significant toxicity to the 661w cells, respectively. By 4 hours PD all cells had high level toxicity, reflecting high levels of cell death from the photo-oxidative damage exposure at this time point (FIG. 26B(i)-(iv)). Comparative analyses across all time points showed that cells incubated with N1, R 100 uM, K 2 uM or G 0.01 mM had progressive levels of toxicity across photo-oxidative damage, cells incubated with SOD or N1/SOD did not. N1/SOD incubated cells had lower cell toxicity measures than untreated controls, shown most clearly at 3 hours photo-oxidative damage (FIG. 26B(v) and (vi)). Similar trends were seen at half dose measures, with R 100 uM having significant baseline toxicity to 661w cells, which was also seen at 2 hours photo-oxidative damage (FIG. 27B(i)-(ii), P<0.05). Other supplementations did not cause toxicity at these time points. Given the high toxicity seen following 4 hours photo-oxidative damage, following 2 hours photo-oxidative damage cells were then recovered for 24 hours to assess long-term effects of supplementations on cell health. Results showed significant toxicity for cells incubated with N1, R 100 uM and G 0.005 mM compared to untreated controls (FIG. 27B(iii)-(iv), P<0.05), but no difference in toxicity for other supplementation groups. Overall, these results indicate that SOD and N1/SOD supplementations may provide some protection against photo-oxidative damage-induced degeneration when utilizing 661w and 661w-EVs, however the protective effects were not as pronounced as RBCs and RBC-EVs.

### Example 24: In Vivo Therapeutic Efficacy of 661w-EV and 661w-EV (N1/SOD) Shows No Protection Against Retinal Degeneration

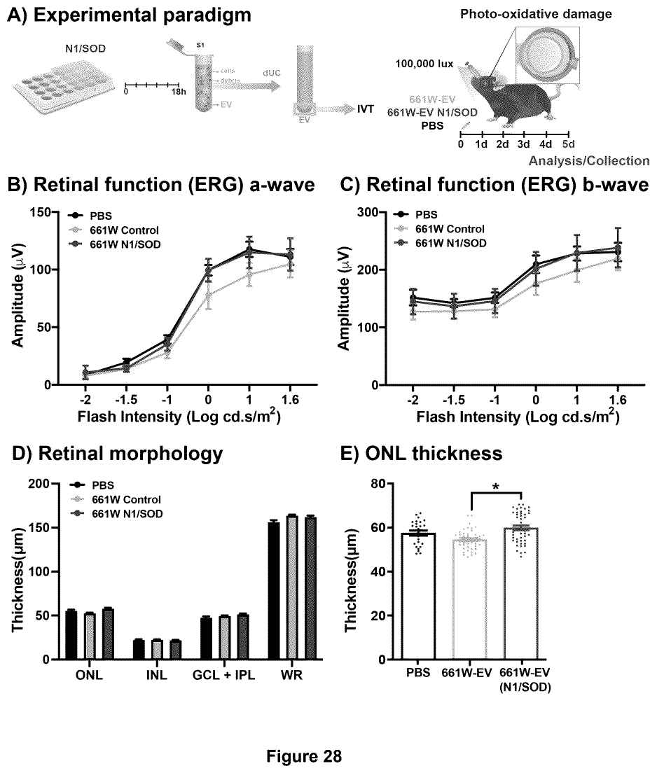

To assess the therapeutic potential of 661w-photoreceptor-like EV on protecting against retinal degeneration, 661w-EV and 661w-EV (N1/SOD) were injected intravitreally at a dose of 5.0×10⁶ EV/mL into mice prior to 5 days of photo-oxidative damage (FIG. 28A). Retinal function as measured by ERG showed no significant protection in any groups compared to PBS-injected controls (FIGS. 28B and C, P<0.05). Retinal thickness measurements showed a significant increase in ONL thickness in 661w-EV (N1/SOD)-treated mice compared to 661w-EV only groups, however no significant improvement was seen compared to PBS-injected controls (FIGS. 28D and E, P<0.05). Overall, 661w-EV with or without supplementation did not confer protection against retinal degeneration however N1/SOD supplementation did confer some protection compared to 661w-EV.

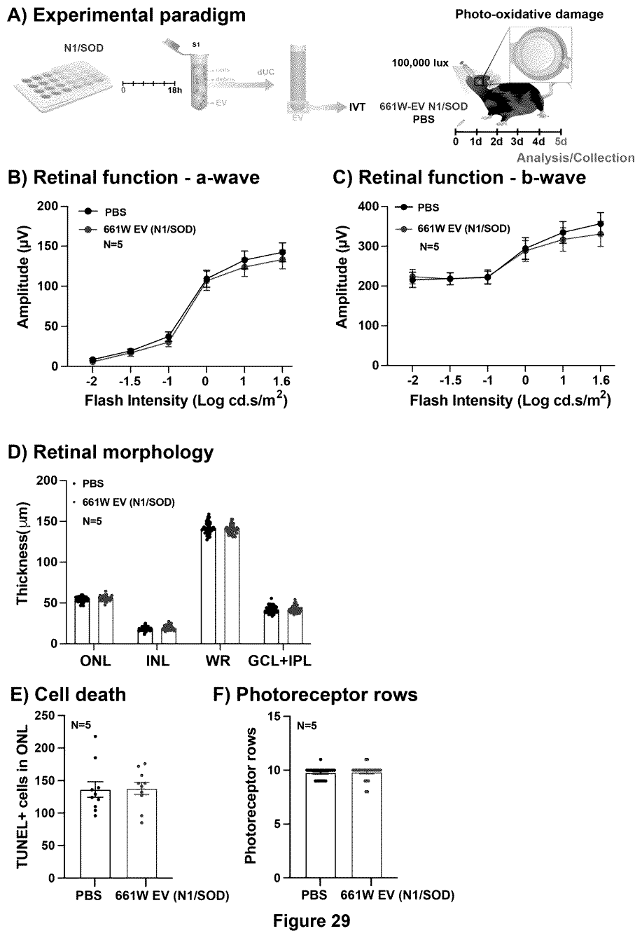

To assess if an increased dose of 661w-EV (N1/SOD) was able to confer protection against photo-oxidative damage-induced degeneration, mice were injected with 2.0×10⁹ 661w-EV (N1/SOD) in 1 μL using intravitreal injection and exposed to 5 days of photo-oxidative damage (FIG. 29A). Following 5 days of photo-oxidative damage, retinal function was measured using ERG, showing no significant difference in a-wave (FIG. 29B) or b-wave responses (FIG. 29C) compared to PBS injected controls (P>0.05). No differences were observed for measures of retinal thickness investigated using optical coherence tomography (FIG. 29D, P>0.05), or cell death (FIGS. 29E-F; P>0.05) as measured using TUNEL assay and photoreceptor row counts. 661w-EV (N1/SOD) overall did not provide therapeutic efficacy.

### Example 25: RBC-EV have a Distinct Proteomic Profile Compared to RBC with N1/SOD Supplementation Conferring Immuno-Modulatory Properties to RBC and RBC-EV

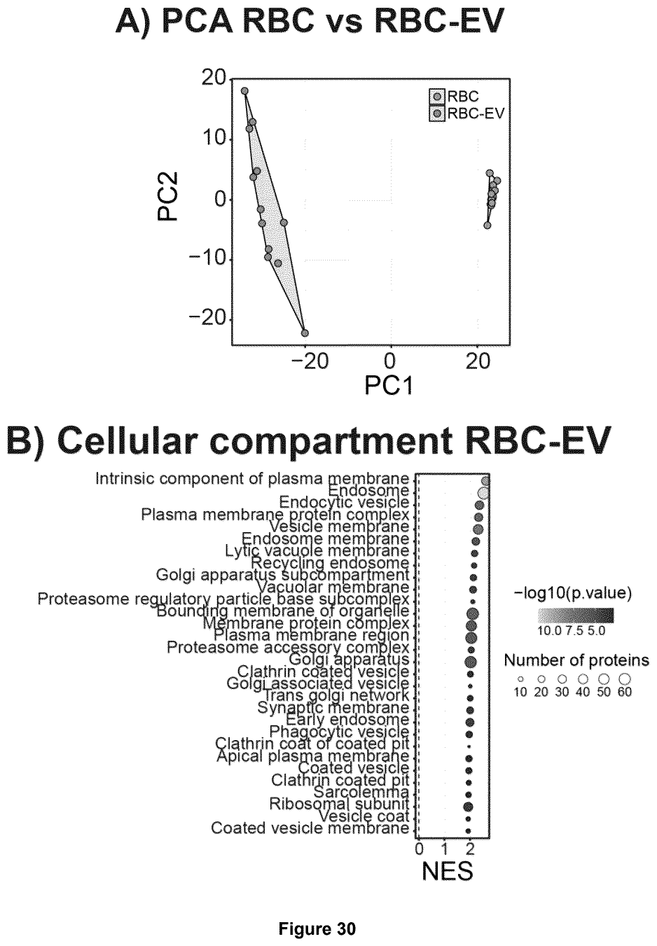

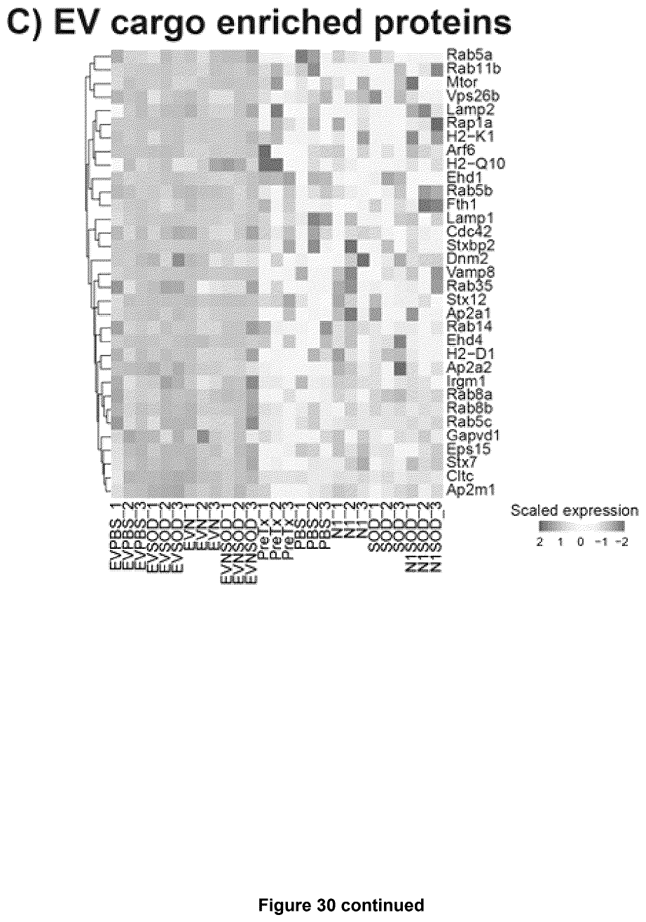

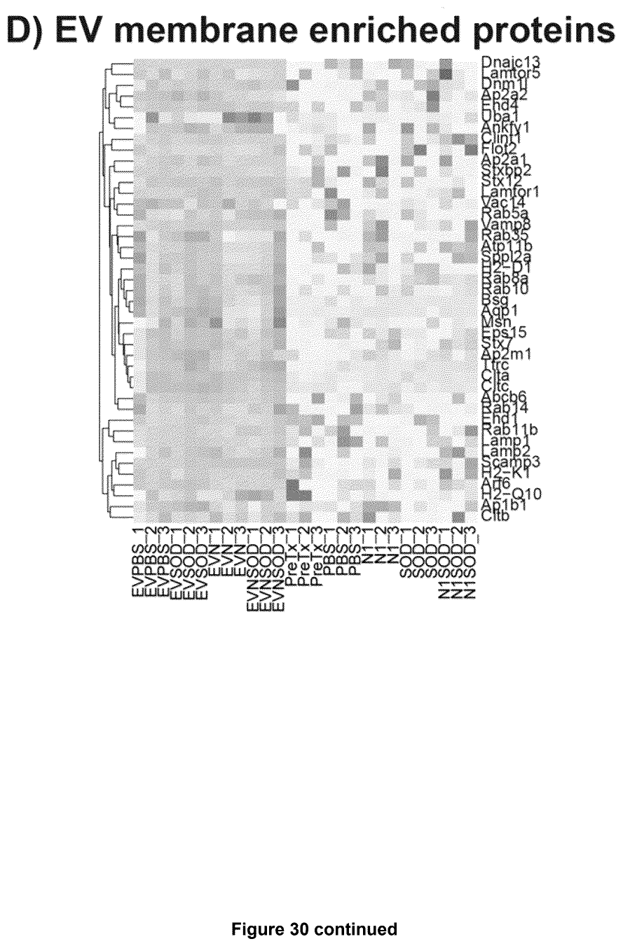

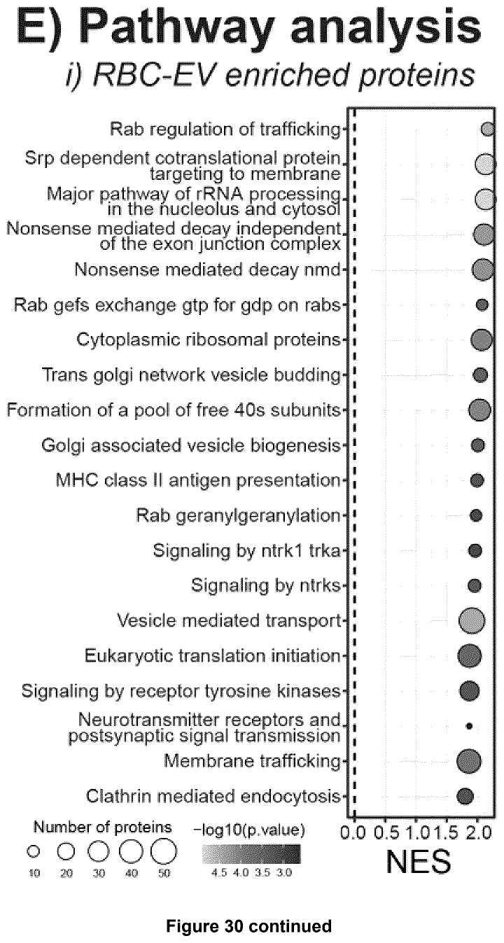

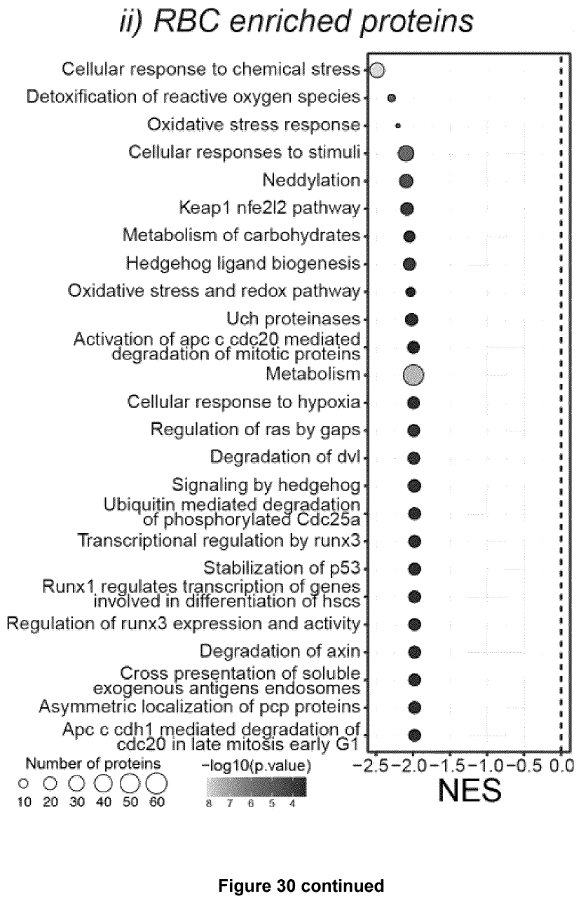

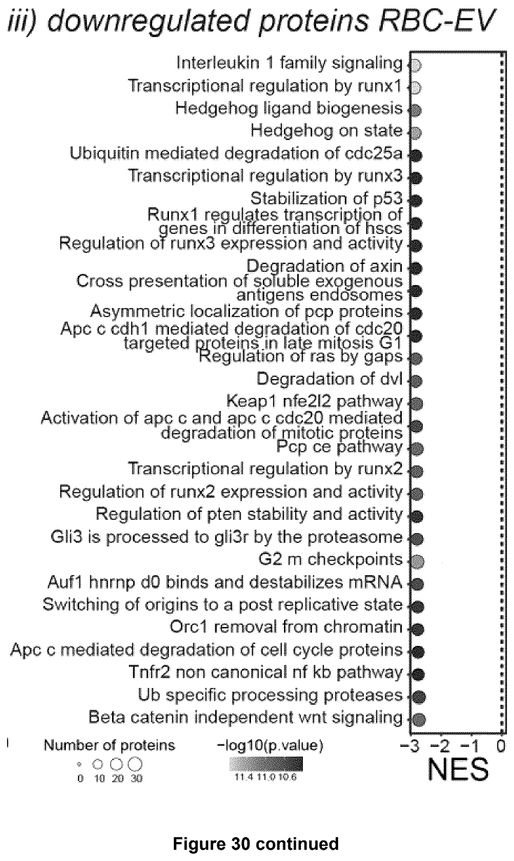

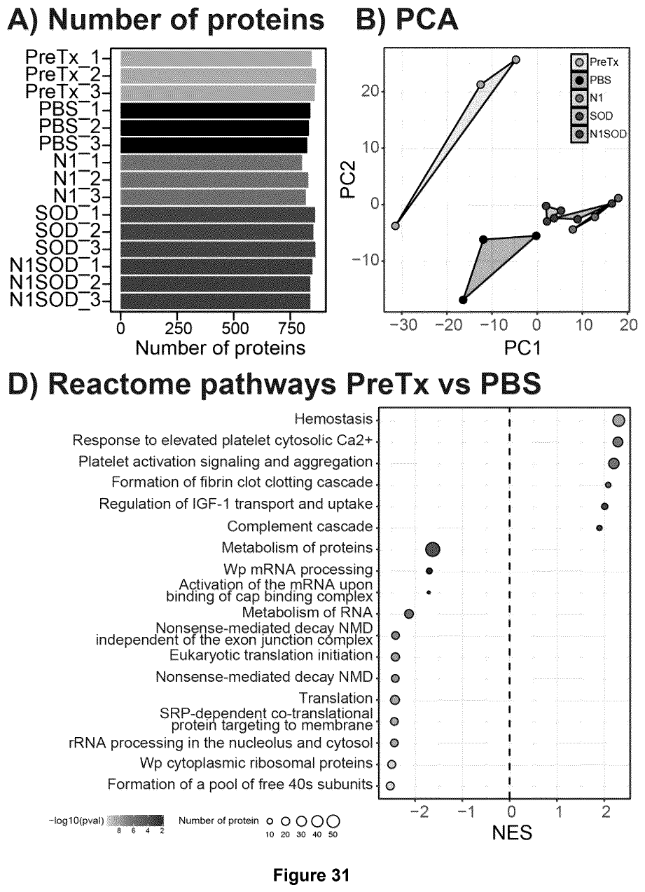

To understand the effect of supplementations on the molecular signature of RBC and RBC-EV, RBC and RBC-EV groups (PBS, N1, SOD and N1/SOD) were sent for LC-MS/MS Tandem Mass Spectrometry. Computational analyses comparing the total protein signature of RBC and RBC-EV revealed a clear proteomic shift between RBC and RBC-EV groups as represented by PCA plot (FIG. 30A). Pathway analysis of RBC-EV proteins was found to be strongly associated with terms such as ‘endosome’, ‘endocytic vesicle’ and ‘vesicle membrane’, supporting the presence of extracellular vesicles in these groups (FIG. 30B). Further, there was a significant enrichment of both known EV cargo proteins (FIG. 30C) and EV membrane proteins (FIG. 30D) in the RBC-EV groups compared to RBC host cells. Pathway analyses of RBC-EV enriched proteins (FIG. 30E (i)), RBC-enriched proteins (FIG. 30E (ii)) further supported known vesicular and red blood cellular functions, respectively. Finally, pathway analysis of downregulated RBC-EV proteins compared to RBC identified decreased enrichment of inflammatory terms such as ‘interleukin 1 family signaling’, and transcriptional pathways (FIG. 30E (iii)), indicating that RBC-EV are likely anti-inflammatory and have reduced transcription regulating proteins compared to their host cells.

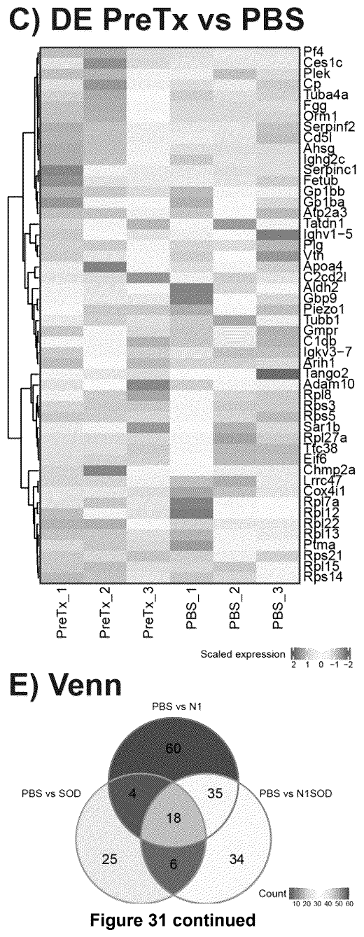

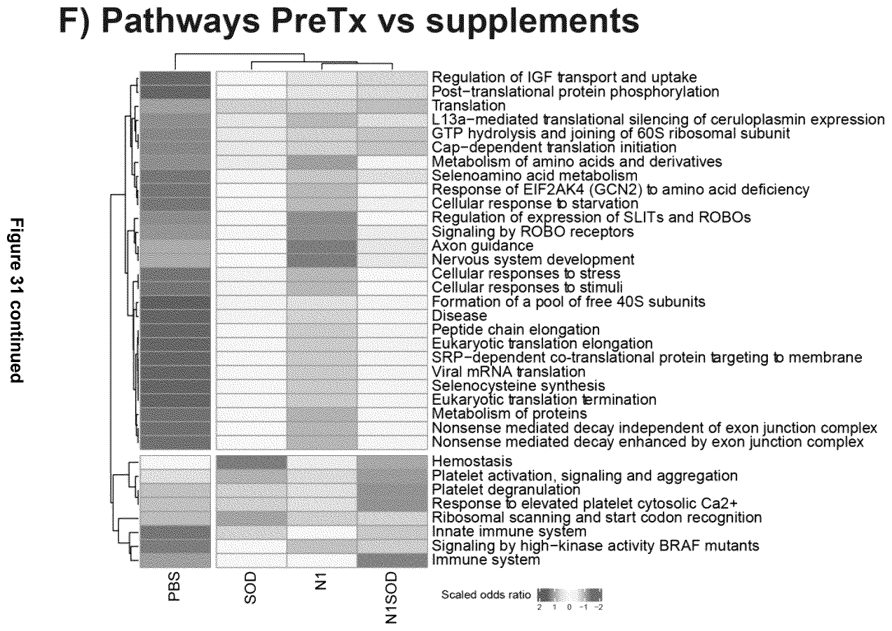

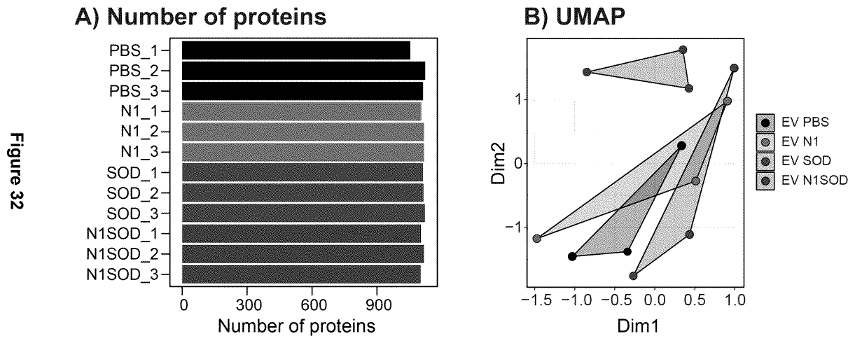

Following, the proteomic signature of RBC was analysed comparing between supplement groups and pre-treatment (Pre-Tx) controls (prior to incubation or supplementation; 0 h). Results demonstrated that there were a similar number of total proteins within each group (FIG. 31A), however between treated groups there was a large amount of variation, in particular compared to Pre-Tx samples, as represented by PCA(FIG. 31B). Analysis of the top differentially expressed proteins between Pre-Tx and RBC (PBS) groups showed that proteins that were enriched in RBC (PBS) samples were associated with pathways controlling hemostasis, clotting, and inflammation, while less enriched proteins were involved in metabolic and translational processes (FIGS. 31C and D). Finally, it was demonstrated that uniquely expressed proteins were present between supplement groups (PBS, N1, SOD and N1/SOD), with RBC (N1) having the most uniquely expressed proteins when compared to RBC (PBS) samples (FIG. 31E). Pathway analysis of proteins in each supplement group compared to Pre-Tx and PBS controls identified that proteins enriched in RBC (N1/SOD) were involved in hemostasis and immune modulation pathways (FIG. 31F).

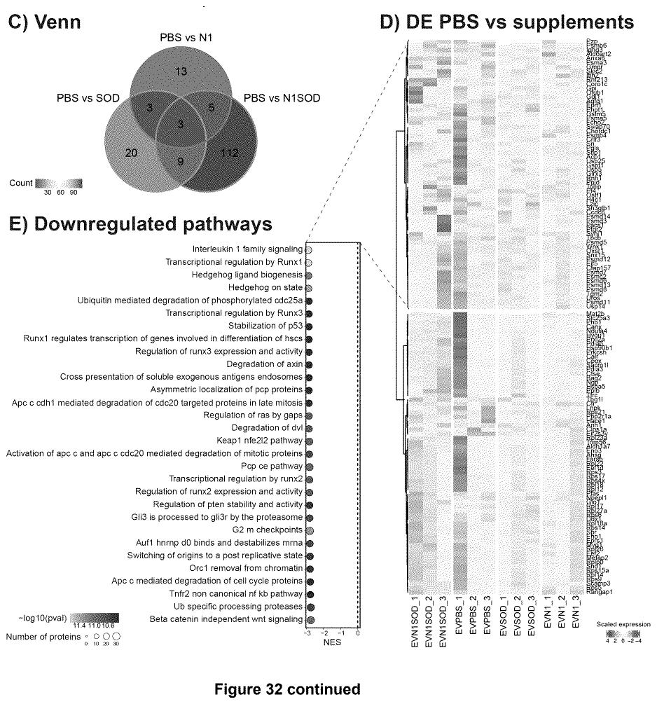

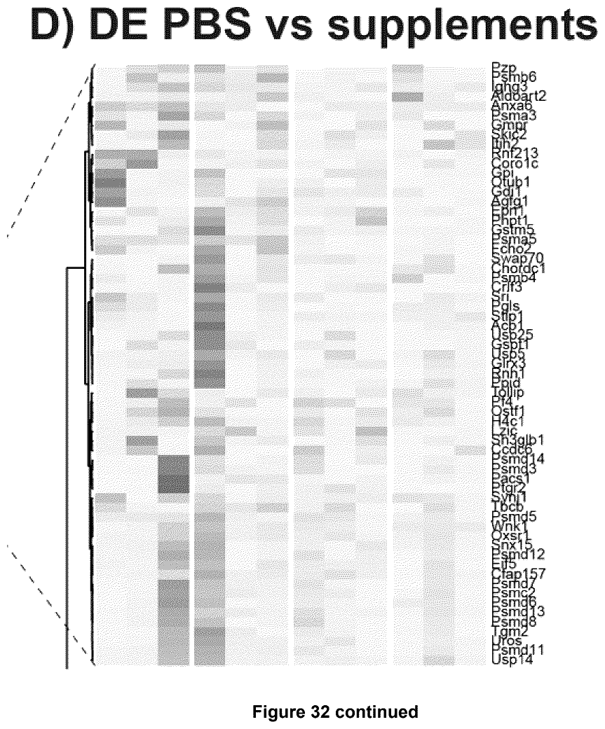

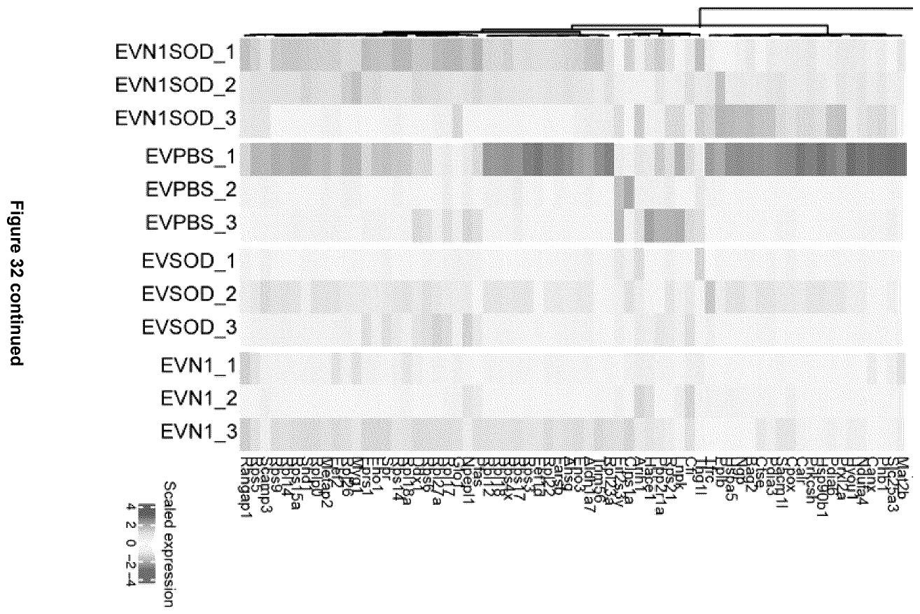

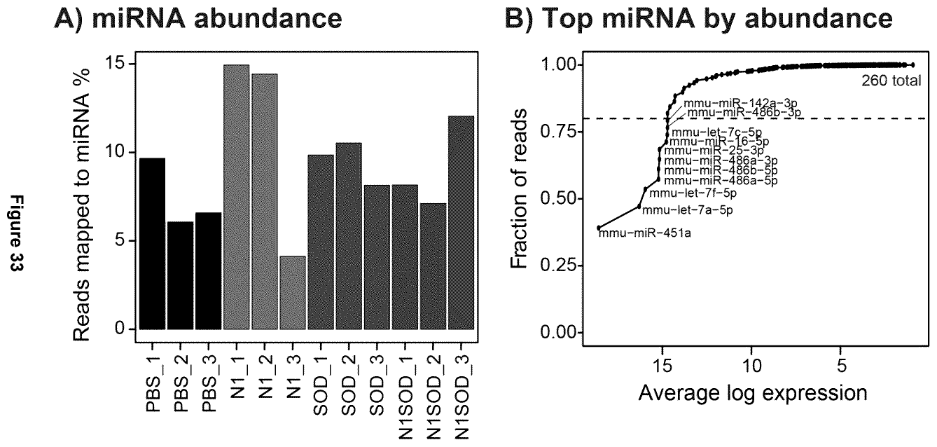

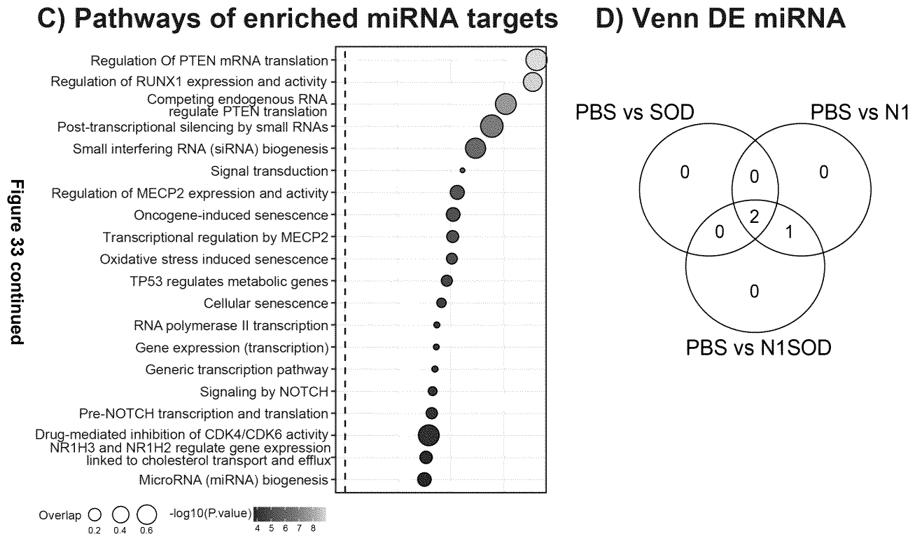

The proteomic composition of RBC-EV samples was also explored, which showed comparable number of proteins within each group (PBS, N1, SOD and N1/SOD) (FIG. 32A). A large variation in protein composition was demonstrated between groups, with RBC-EV (N1/SOD) having the most variable expression compared to RBC-EV (PBS), RBC-EV (N1) and RBC-EV (SOD) groups (FIG. 32B), which was also reflected in the high number of unique proteins found in this group (FIG. 32C). Compared to RBC-EV (PBS), RBC-EV (N1), and RBC-EV (SOD) groups, RBC-EV (N1/SOD) were also shown to have a population of significantly downregulated proteins (FIG. 32D), which were found to be associated with inflammatory pathways (FIG. 32E) including ‘interleukin 1 family signalling’ ‘non-canonical NfkB pathway’ and ‘keap1/nef212 pathways’ as well as transcriptional regulation of inflammatory, apoptotic, mitotic, and hematopoietic pathways. The full list of differentially expressed proteins is described below in Table 1. In Table 1, proteins having a positive Fold Change (log 2) value are those that are upregulated compared to the control, and proteins having a negative Fold Change (log 2) value are those that are downregulated compared to the control. The full list of pathways in which the differentially expressed proteins belong to, interact with, or mediate is described below in Table 2. In Table 2, pathways having a positive Fold Change value are pathways that are upregulated compared to the control, and pathways having a negative Fold Change value are those that are downregulated compared to the control.

TABLE 1
String ID, gene name and full name of proteins differentially expressed
in RBC EV (N1/SOD) in comparison to RBC EV (PBS) control.

| | Fold | P. Value | | |
|---|---|---|---|---|
| | Change | (N1SOD vs | | |
| Name | (log2) | PBS) | STRING or MGI | Full Name |
| Arih1 | 5.201 | 0.032 | 10090.ENSMUSP00000126531 | E3 ubiquitin protein ligase 1. |
| Rpl22 | 3.063 | 0.015 | 10090.ENSMUSP00000118787 | 60S ribosomal protein L22. |
| Cfi | 3.053 | 0.039 | 10090.ENSMUSP00000077074 | Complement factor I heavy chain |
| Lnpk | 2.893 | 0.002 | 10090.ENSMUSP00000066891 | Endoplasmic reticulum junction |
| | | | | formation protein lunapark |
| Rps17 | 2.780 | 0.030 | 10090.ENSMUSP00000079628 | 40S ribosomal protein S17 |
| Rpl23a | 2.614 | 0.016 | 10090.ENSMUSP00000099541 | 60S ribosomal protein L23a |
| Rps21 | 2.454 | 0.037 | 10090.ENSMUSP00000058432 | 40S ribosomal protein S21 |
| Cpox | 2.240 | 0.010 | 10090.ENSMUSP00000055455 | Oxygen-dependent |
| | | | | coproporphyrinogen-III oxidase, |
| | | | | mitochondrial |
| Metap2 | 2.104 | 0.025 | 10090.ENSMUSP00000048285 | Methionine aminopeptidase 2 |
| Farsb | 2.010 | 0.046 | 10090.ENSMUSP00000129828 | Phenylalanine--tRNA ligase beta |
| | | | | subunit. |
| Rpl18 | 1.952 | 0.035 | 10090.ENSMUSP00000147816 | 60S ribosomal protein L18 |
| Myg1 | 1.879 | 0.050 | 10090.ENSMUSP00000109312 | UPF0160 protein MYG1, |
| | | | | mitochondrial. |
| Rps3 | 1.872 | 0.046 | 10090.ENSMUSP00000032998 | 40S ribosomal protein S3 |
| Rps15a | 1.861 | 0.019 | 10090.ENSMUSP00000119975 | 40S ribosomal protein S15a |
| Hsp90b1 | 1.830 | 0.031 | 10090.ENSMUSP00000020238 | Endoplasmin |
| Phb1 | 1.823 | 0.029 | MGI: 97572 | Prohibitin 1 |
| Eef1d | 1.815 | 0.040 | 10090.ENSMUSP00000105602 | Elongation factor 1-delta |
| Ddx1 | 1.799 | 0.021 | 10090.ENSMUSP00000065987 | ATP-dependent RNA helicase DDX1 |
| Rps4x | 1.777 | 0.039 | 10090.ENSMUSP00000033683 | 40S ribosomal protein S4, X isoform |
| Eif2s3y | 1.676 | 0.036 | 10090.ENSMUSP00000088736 | Eukaryotic translation initiation factor |
| | | | | 2 subunit 3, Y-linked |
| Ahsg | 1.652 | 0.036 | 10090.ENSMUSP00000023583 | Alpha-2-HS-glycoprotein |
| Rpl12 | 1.636 | 0.045 | 10090.ENSMUSP00000117461 | 60S ribosomal protein L12 |
| Ndufa4 | 1.553 | 0.024 | 10090.ENSMUSP00000144932 | Cytochrome c oxidase subunit |
| | | | | NDUFA4 |
| Sacm1l | 1.517 | 0.011 | 10090.ENSMUSP00000026270 | Phosphatidylinositide phosphatase |
| | | | | SAC1 |
| Rps5 | 1.511 | 0.013 | 10090.ENSMUSP00000104179 | 40S ribosomal protein S5, N-terminally |
| | | | | processed |
| Rpl18a | 1.451 | 0.038 | 10090.ENSMUSP00000058368 | 60S ribosomal protein L18a. |
| Rpl14 | 1.428 | 0.039 | 10090.ENSMUSP00000131489 | 60S ribosomal protein L14 |
| Calr | 1.410 | 0.029 | 10090.ENSMUSP00000003912 | Calreticulin |
| Rpl26 | 1.408 | 0.043 | 10090.ENSMUSP00000129072 | 60S ribosomal protein L26 |
| Rplp0 | 1.325 | 0.040 | 10090.ENSMUSP00000083705 | 60S acidic ribosomal protein P0 |
| Hyou1 | 1.324 | 0.038 | 10090.ENSMUSP00000123700 | Hypoxia up-regulated protein 1 |
| Eno3 | 1.294 | 0.047 | 10090.ENSMUSP00000104188 | Beta-enolase |
| Ctse | 1.286 | 0.026 | 10090.ENSMUSP00000073072 | Cathepsin E |
| Aldh1a7 | 1.283 | 0.035 | 10090.ENSMUSP00000025656 | Aldehyde dehydrogenase, cytosolic 1 |
| Pdia6 | 1.248 | 0.017 | 10090.ENSMUSP00000052912 | Protein disulfide-isomerase A6 |
| Prkcsh | 1.218 | 0.019 | 10090.ENSMUSP00000110987 | Glucosidase 2 subunit beta |
| Clns1a | 1.215 | 0.016 | 10090.ENSMUSP00000026506 | Methylosome subunit pICln |
| Rps6 | 1.193 | 0.020 | 10090.ENSMUSP00000099878 | 40S ribosomal protein S6 |
| Hace1 | 1.187 | 0.001 | 10090.ENSMUSP00000039206 | E3 ubiquitin-protein ligase HACE1 |
| Rpl17 | 1.187 | 0.035 | 10090.ENSMUSP00000157556 | 60S ribosomal protein L17 |
| Rpl27a | 1.166 | 0.037 | 10090.ENSMUSP00000123410 | 60S ribosomal protein L27a. |
| Eprs1 | 1.147 | 0.047 | 10090.ENSMUSP00000045841 | Bifunctional glutamate/proline--tRNA |
| | | | | ligase |
| Prxl2a | 1.145 | 0.010 | 10090.ENSMUSP00000112377 | Peroxiredoxin-like 2A |
| Trim56 | 1.141 | 0.031 | 10090.ENSMUSP00000058109 | E3 ubiquitin-protein ligase TRIM56 |
| Canx | 1.115 | 0.049 | 10090.ENSMUSP00000137440 | Calnexin |
| Npepl1 | 1.109 | 0.036 | 10090.ENSMUSP00000042808 | Probable aminopeptidase NPEPL1 |
| Pdia3 | 1.093 | 0.017 | 10090.ENSMUSP00000028683 | Protein disulfide-isomerase A3. |
| Ppib | 1.085 | 0.007 | 10090.ENSMUSP00000034947 | Peptidyl-prolyl cis-trans isomerase B |
| Eef2 | 1.020 | 0.037 | 10090.ENSMUSP00000046101 | Elongation factor 2 |
| Spr | 1.012 | 0.038 | 10090.ENSMUSP00000048111 | Sepiapterin reductase |
| Ngp | 1.010 | 0.023 | 10090.ENSMUSP00000035061 | Neutrophilic granule protein |
| Hspa5 | 1.008 | 0.042 | 10090.ENSMUSP00000028222 | Endoplasmic reticulum chaperone BiP |
| Bag2 | 0.978 | 0.019 | 10090.ENSMUSP00000042009 | BAG family molecular chaperone |
| | | | | regulator 2 |
| Snd1 | 0.974 | 0.032 | 10090.ENSMUSP00000001460 | Staphylococcal nuclease domain- |
| | | | | containing protein 1 |
| Rangap1 | 0.901 | 0.032 | 10090.ENSMUSP00000057771 | Ran GTPase-activating protein 1 |
| Eno1 | 0.895 | 0.044 | 10090.ENSMUSP00000079727 | Enolase 1, alpha non-neuron. |
| Rps14 | 0.875 | 0.048 | 10090.ENSMUSP00000157948 | 40S ribosomal protein S14 |
| Tfrc | 0.836 | 0.018 | 10090.ENSMUSP00000023486 | Transferrin receptor protein 1 |
| Thg1l | 0.815 | 0.023 | 10090.ENSMUSP00000011398 | Probable tRNA(His) |
| | | | | guanylyltransferase |
| Pfas | 0.788 | 0.029 | 10090.ENSMUSP00000021282 | Phosphoribosylformylglycinamidine |
| | | | | synthase |
| Ppp2r1a | 0.601 | 0.032 | 10090.ENSMUSP00000007708 | Serine/threonine-protein phosphatase |
| | | | | 2A 65 kDa regulatory subunit A alpha |
| | | | | isoform |
| Glo1 | 0.547 | 0.038 | 10090.ENSMUSP00000158296 | Lactoylglutathione lyase |
| Scamp3 | 0.519 | 0.030 | 10090.ENSMUSP00000112846 | Secretory carrier-associated membrane |
| | | | | protein 3 |
| Fcho2 | −0.411 | 0.048 | 10090.ENSMUSP00000042959 | F-BAR domain only protein 2 |
| Crlf3 | −0.487 | 0.045 | 10090.ENSMUSP00000060028 | Cytokine receptor-like factor 3 |
| Psma5 | −0.516 | 0.011 | 10090.ENSMUSP00000088057 | Proteasome subunit alpha type-5 |
| Gspt1 | −0.530 | 0.044 | 10090.ENSMUSP00000078940 | Eukaryotic peptide chain release factor |
| | | | | GTP-binding subunit ERF3A |
| Psmb4 | −0.567 | 0.030 | 10090.ENSMUSP00000005923 | Proteasome subunit beta type-4 |
| Acp1 | −0.596 | 0.043 | 10090.ENSMUSP00000106509 | Low molecular weight phosphotyrosine |
| | | | | protein phosphatase |
| Ccdc6 | −0.624 | 0.046 | 10090.ENSMUSP00000123374 | Coiled-coil domain-containing protein |
| | | | | 6. |
| Uros | −0.627 | 0.041 | 10090.ENSMUSP00000033276 | Uroporphyrinogen-III synthase |
| Psmd3 | −0.647 | 0.039 | 10090.ENSMUSP00000017365 | 26S proteasome non-ATPase regulatory |
| | | | | subunit 3 |
| Tgm2 | −0.647 | 0.034 | 10090.ENSMUSP00000099411 | Protein-glutamine gamma- |
| | | | | glutamyltransferase 2 |
| Psmc2 | −0.652 | 0.042 | 10090.ENSMUSP00000030769 | 26S proteasome regulatory subunit 7 |
| Psma3 | −0.660 | 0.007 | 10090.ENSMUSP00000125548 | Proteasome subunit alpha type-3 |
| Glrx3 | −0.669 | 0.029 | 10090.ENSMUSP00000066621 | Glutaredoxin-3 |
| Rnh1 | −0.669 | 0.025 | 10090.ENSMUSP00000147928 | Ribonuclease inhibitor |
| Ppid | −0.671 | 0.039 | 10090.ENSMUSP00000029382 | Peptidyl-prolyl cis-trans isomerase D |
| Usp25 | −0.671 | 0.028 | 10090.ENSMUSP00000023580 | Ubiquitin carboxyl-terminal hydrolase |
| | | | | 25 |
| Gmpr | −0.677 | 0.018 | 10090.ENSMUSP00000000260 | GMP reductase 1 |
| Usp5 | −0.691 | 0.017 | 10090.ENSMUSP00000041299 | Ubiquitin carboxyl-terminal hydrolase |
| | | | | 5 |
| Pgls | −0.701 | 0.038 | 10090.ENSMUSP00000034264 | 6-phosphogluconolactonase |
| Ostf1 | −0.738 | 0.016 | 10090.ENSMUSP00000025631 | Osteoclast-stimulating factor 1 |
| Psmb6 | −0.744 | 0.007 | 10090.ENSMUSP00000018430 | Proteasome subunit beta type-6 |
| Eif5 | −0.745 | 0.014 | 10090.ENSMUSP00000126825 | Eukaryotic translation initiation factor |
| | | | | 5 |
| Tbcb | −0.755 | 0.010 | 10090.ENSMUSP00000006254 | Tubulin-folding cofactor B |
| Oxsr1 | −0.756 | 0.013 | 10090.ENSMUSP00000042155 | Serine/threonine-protein kinase OSR1 |
| Stip1 | −0.778 | 0.033 | 10090.ENSMUSP00000025918 | Stress-induced-phosphoprotein 1 |
| Usp14 | −0.779 | 0.021 | 10090.ENSMUSP00000089728 | Ubiquitin carboxyl-terminal hydrolase |
| | | | | 14 |
| Psmd5 | −0.787 | 0.012 | 10090.ENSMUSP00000028225 | 26S proteasome non-ATPase regulatory |
| | | | | subunit 5 |
| Tollip | −0.792 | 0.034 | 10090.ENSMUSP00000001950 | Toll-interacting protein |
| Psmd8 | −0.809 | 0.023 | 10090.ENSMUSP00000051657 | 26S proteasome non-ATPase regulatory |
| | | | | subunit 8 |
| Gpi | −0.846 | 0.047 | 10090.ENSMUSP00000049355 | Glucose-6-phosphate isomerase |
| Otub1 | −0.847 | 0.049 | 10090.ENSMUSP00000025679 | Ubiquitin thioesterase OTUB1 |
| Sri | −0.850 | 0.007 | 10090.ENSMUSP00000118221 | Sorcin |
| Agfg1 | −0.851 | 0.015 | 10090.ENSMUSP00000140170 | Arf-GAP domain and FG repeat- |
| | | | | containing protein 1 |
| Cfap157 | −0.859 | 0.016 | 10090.ENSMUSP00000099877 | Cilia- and flagella-associated protein |
| | | | | 157 |
| Psmd6 | −0.863 | 0.024 | 10090.ENSMUSP00000022256 | 26S proteasome non-ATPase regulatory |
| | | | | subunit 6 |
| Chordc1 | −0.881 | 0.019 | 10090.ENSMUSP00000001825 | Cysteine and histidine-rich domain- |
| | | | | containing protein 1 |
| Phpt1 | −0.883 | 0.045 | 10090.ENSMUSP00000037417 | 14 kDa phosphohistidine phosphatase |
| Psmd7 | −0.898 | 0.019 | 10090.ENSMUSP00000041968 | 26S proteasome non-ATPase regulatory |
| | | | | subunit 7 |
| Psmd11 | −0.900 | 0.011 | 10090.ENSMUSP00000017572 | 26S proteasome non-ATPase regulatory |
| | | | | subunit 11 |
| Psmd12 | −0.916 | 0.014 | 10090.ENSMUSP00000021063 | 26S proteasome non-ATPase regulatory |
| | | | | subunit 12 |
| Psmd13 | −0.944 | 0.014 | 10090.ENSMUSP00000026560 | 26S proteasome non-ATPase regulatory |
| | | | | subunit 13 |
| Gstm5 | −0.980 | 0.027 | 10090.ENSMUSP00000004134 | Glutathione S-transferase Mu 5 |
| Synj1 | −1.021 | 0.020 | 10090.ENSMUSP00000113308 | Synaptojanin-1 |
| Sh3glb1 | −1.030 | 0.020 | 10090.ENSMUSP00000143312 | Endophilin-B1 |
| Swap70 | −1.078 | 0.016 | 10090.ENSMUSP00000033325 | Switch-associated protein 70 |
| H4c1 | −1.088 | 0.036 | 10090.ENSMUSP00000100029 | H4 clustered histone 1. |
| Pzp | −1.102 | 0.005 | 10090.ENSMUSP00000107760 | Alpha-2-macroglobulin 165 kDa |
| | | | | subunit |
| Psmd14 | −1.120 | 0.033 | 10090.ENSMUSP00000028278 | 26S proteasome non-ATPase regulatory |
| | | | | subunit 14 |
| Wnk1 | −1.138 | 0.012 | 10090.ENSMUSP00000086017 | Serine/threonine-protein kinase WNK1 |
| Gdi1 | −1.156 | 0.003 | 10090.ENSMUSP00000015435 | Rab GDP dissociation inhibitor alpha |
| Pf4 | −1.176 | 0.025 | 10090.ENSMUSP00000031320 | Platelet factor 4 |
| Snx15 | −1.311 | 0.004 | 10090.ENSMUSP00000025702 | Sorting nexin-15 |
| Coro1c | −1.359 | 0.013 | 10090.ENSMUSP00000004646 | Coronin-1C |
| Ptgr2 | −1.503 | 0.030 | 10090.ENSMUSP00000115704 | Prostaglandin reductase 2 |
| Aldoart2 | −1.526 | 0.003 | 10090.ENSMUSP00000079022 | Fructose-bisphosphate aldolase |
| Ighg3 | −1.687 | 0.001 | MGI: 2144790 | Immunoglobulin heavy constant |
| | | | | gamma 3 |
| Lzic | −1.714 | 0.041 | 10090.ENSMUSP00000030842 | Protein LZIC. |
| Epn1 | −1.729 | 0.003 | 10090.ENSMUSP00000096445 | Epsin-1 |
| Pacs1 | −1.871 | 0.023 | 10090.ENSMUSP00000025786 | Phosphofurin acidic cluster sorting |
| | | | | protein 1 |
| Skic2 | −3.081 | 0.006 | MGI: 1099835 | SKI2 subunit of superkiller complex |
| Kyat3 | −5.919 | 0.049 | 10090.ENSMUSP00000101825 | Kynurenine--oxoglutarate transaminase |
| | | | | 3 |
| Rnf213 | −6.678 | 0.011 | 10090.ENSMUSP00000091429 | E3 ubiquitin-protein ligase RNF213 |
| Anxa6 | −8.011 | 0.000 | 10090.ENSMUSP00000104511 | Annexin A6 |

TABLE 2
Pathway ID and fold change in RBC EV (N1/SOD) in comparison to RBC EV (PBS) control.

| | | Fold Change |
|---|---|---|
| | | (Normalised |
| | | Enrichment |
| Pathway ID (Reactome) | P Value | Score) |
| REACTOME_MAJOR_PATHWAY_OF_RRNA_PROCESSING_{—} | 1.021E−17 | 3.237 |
| IN_THE_NUCLEOLUS_AND_CYTOSOL | | |
| REACTOME_SRP_DEPENDENT_COTRANSLATIONAL_{—} | 1.021E−17 | 3.237 |
| PROTEIN_TARGETING_TO_MEMBRANE | | |
| REACTOME_NONSENSE_MEDIATED_DECAY_NMD | 6.395E−17 | 3.190 |
| REACTOME_NONSENSE_MEDIATED_DECAY_NMD_{—} | 2.202E−16 | 3.155 |
| INDEPENDENT_OF_THE_EXON_JUNCTION_COMPLEX_EJC | | |
| REACTOME_FORMATION_OF_A_POOL_OF_FREE_40S_{—} | 2.108E−14 | 3.012 |
| SUBUNITS | | |
| REACTOME_TRANSLATION | 2.651E−12 | 2.763 |
| REACTOME_EUKARYOTIC_TRANSLATION_INITIATION | 3.033E−11 | 2.734 |
| REACTOME_ACTIVATION_OF_THE_MRNA_UPON_BINDING_{—} | 1.380E−04 | 2.095 |
| OF_THE_CAP_BINDING_COMPLEX_AND_EIFS_AND_{—} | | |
| SUBSEQUENT_BINDING_TO_43S | | |
| REACTOME_ANTIGEN_PRESENTATION_FOLDING_{—} | 2.362E−03 | 1.970 |
| ASSEMBLY_AND_PEPTIDE_LOADING_OF_CLASS_I_MHC | | |
| REACTOME_PHASE_I_FUNCTIONALIZATION_OF_{—} | 1.200E−02 | 1.783 |
| COMPOUNDS | | |
| REACTOME_SIGNALING_BY_INSULIN_RECEPTOR | 1.120E−02 | 1.750 |
| REACTOME_COPI_DEPENDENT_GOLGI_TO_ER_{—} | 2.882E−02 | 1.674 |
| RETROGRADE_TRAFFIC | | |
| REACTOME_METABOLISM_OF_RNA | 3.834E−03 | 1.590 |
| REACTOME_THE_CITRIC_ACID_TCA_CYCLE_AND_{—} | 2.017E−02 | 1.583 |
| RESPIRATORY_ELECTRON_TRANSPORT | | |
| REACTOME_SENSORY_PERCEPTION | 4.501E−02 | 1.517 |
| REACTOME_VISUAL_PHOTOTRANSDUCTION | 4.501E−02 | 1.517 |
| REACTOME_METABOLISM_OF_PROTEINS | 3.507E−03 | 1.482 |
| REACTOME_METABOLISM | 1.442E−02 | −1.368 |
| REACTOME_ADAPTIVE_IMMUNE_SYSTEM | 1.064E−02 | −1.482 |
| REACTOME_INNATE_IMMUNE_SYSTEM | 2.740E−03 | −1.549 |
| REACTOME_NEUTROPHIL_DEGRANULATION | 6.050E−03 | −1.574 |
| REACTOME_ACTIVATION_OF_BAD_AND_{—} | 4.817E−02 | −1.581 |
| TRANSLOCATION_TO_MITOCHONDRIA | | |
| REACTOME_ACTIVATION_OF_BH3_ONLY_PROTEINS | 4.817E−02 | −1.581 |
| REACTOME_POST_TRANSLATIONAL_PROTEIN_{—} | 8.705E−04 | −1.605 |
| MODIFICATION | | |
| REACTOME_CHK1_CHK2_CDS1_MEDIATED_{—} | 2.865E−02 | −1.615 |
| INACTIVATION_OF_CYCLIN_B_CDK1_COMPLEX | | |
| REACTOME_DNA_REPAIR | 2.605E−02 | −1.666 |
| REACTOME_CLATHRIN_MEDIATED_ENDOCYTOSIS | 8.809E−03 | −1.698 |
| REACTOME_CELLULAR_RESPONSES_TO_STIMULI | 8.511E−04 | −1.714 |
| REACTOME_TRANSPORT_OF_SMALL_MOLECULES | 8.616E−04 | −1.719 |
| REACTOME_G2_M_DNA_DAMAGE_CHECKPOINT | 1.072E−02 | −1.767 |
| REACTOME_CLASS_I_MHC_MEDIATED_ANTIGEN_{—} | 3.025E−04 | −1.872 |
| PROCESSING_PRESENTATION | | |
| REACTOME_CELL_CYCLE | 1.163E−04 | −1.883 |
| REACTOME_CYTOKINE_SIGNALING_IN_IMMUNE_SYSTEM | 5.338E−06 | −2.133 |
| REACTOME_M_PHASE | 3.037E−06 | −2.168 |
| REACTOME_GENE_EXPRESSION_TRANSCRIPTION | 4.786E−07 | −2.179 |
| REACTOME_ANTIGEN_PROCESSING_UBIQUITINATION_{—} | 9.530E−07 | −2.212 |
| PROTEASOME_DEGRADATION | | |
| REACTOME_MITOTIC_G2_G2_M_PHASES | 3.493E−06 | −2.220 |
| REACTOME_GENERIC_TRANSCRIPTION_PATHWAY | 2.008E−07 | −2.255 |
| REACTOME_MAPK_FAMILY_SIGNALING_CASCADES | 1.372E−07 | −2.262 |
| REACTOME_INTRACELLULAR_SIGNALING_BY_SECOND_{—} | 6.210E−07 | −2.310 |
| MESSENGERS | | |
| REACTOME_MITOTIC_METAPHASE_AND_ANAPHASE | 3.000E−07 | −2.361 |
| REACTOME_SEPARATION_OF_SISTER_CHROMATIDS | 6.153E−08 | −2.424 |
| REACTOME_SIGNALING_BY_INTERLEUKINS | 2.689E−08 | −2.427 |
| REACTOME_METABOLISM_OF_AMINO_ACIDS_AND_{—} | 4.396E−08 | −2.452 |
| DERIVATIVES | | |
| REACTOME_CELLULAR_RESPONSE_TO_CHEMICAL_STRESS | 6.997E−09 | −2.455 |
| REACTOME_ANTIGEN_PROCESSING_CROSS_PRESENTATION | 1.125E−07 | −2.473 |
| REACTOME_CELL_CYCLE_CHECKPOINTS | 8.777E−09 | −2.485 |
| REACTOME_FC_EPSILON_RECEPTOR_FCERI_SIGNALING | 8.991E−09 | −2.528 |
| REACTOME_SIGNALING_BY_WNT | 2.185E−09 | −2.546 |
| REACTOME_THE_ROLE_OF_GTSE1_IN_G2_M_{—} | 6.079E−09 | −2.580 |
| PROGRESSION_AFTER_G2_CHECKPOINT | | |
| REACTOME_G1_S_TRANSITION | 7.364E−09 | −2.589 |
| REACTOME_MITOTIC_G1_PHASE_AND_G1_S_TRANSITION | 7.364E−09 | −2.589 |
| REACTOME_HEDGEHOG_OFF_STATE | 1.675E−09 | −2.597 |
| REACTOME_TCF_DEPENDENT_SIGNALING_IN_RESPONSE_{—} | 4.465E−09 | −2.600 |
| TO_WNT | | |
| REACTOME_NEDDYLATION | 5.379E−10 | −2.613 |
| REACTOME_SIGNALING_BY_THE_B_CELL_RECEPTOR_BCR | 3.147E−09 | −2.618 |
| REACTOME_ABC_FAMILY_PROTEINS_MEDIATED_TRANSPORT | 1.826E−09 | −2.619 |
| REACTOME_DEUBIQUITINATION | 1.061E−10 | −2.622 |
| REACTOME_DEGRADATION_OF_BETA_CATENIN_BY_THE_{—} | 2.206E−09 | −2.623 |
| DESTRUCTION_COMPLEX | | |
| REACTOME_TCR_SIGNALING | 1.939E−09 | −2.633 |
| REACTOME_DOWNSTREAM_TCR_SIGNALING | 9.369E−10 | −2.657 |
| REACTOME_SIGNALING_BY_HEDGEHOG | 1.858E−10 | −2.675 |
| REACTOME_REGULATION_OF_MRNA_STABILITY_BY | 5.967E−10 | 2.678 |
| PROTEINS_THAT_BIND_AU_RICH_ELEMENTS | | |
| REACTOME_DNA_REPLICATION | 5.523E−10 | −2.681 |
| REACTOME_PTEN_REGULATION | 4.801E−10 | −2.689 |
| REACTOME_CELLULAR_RESPONSE_TO_HYPOXIA | 3.634E−10 | −2.693 |
| REACTOME_S_PHASE | 2.283E−10 | −2.710 |
| REACTOME_METABOLISM_OF_POLYAMINES | 1.222E−10 | −2.716 |
| REACTOME_UB_SPECIFIC_PROCESSING_PROTEASES | 2.606E−11 | −2.726 |
| REACTOME_MAPK6_MAPK4_SIGNALING | 1.372E−10 | −2.726 |
| REACTOME_FCERI_MEDIATED_NF_KB_ACTIVATION | 1.549E−10 | −2.728 |
| REACTOME_BETA_CATENIN_INDEPENDENT_WNT_{—} | 3.467E−11 | −2.731 |
| SIGNALING | | |
| REACTOME_UCH_PROTEINASES | 7.988E−11 | −2.735 |
| REACTOME_TNFR2_NON_CANONICAL_NF_KB_PATHWAY | 7.720E−11 | −2.737 |
| REACTOME_DNA_REPLICATION_PRE_INITIATION | 7.323E−11 | −2.739 |
| REACTOME_APC_C_MEDIATED_DEGRADATION_OF_CELL_{—} | 7.006E−11 | −2.740 |
| CYCLE_PROTEINS | | |
| REACTOME_CLEC7A_DECTIN_1_SIGNALING | 9.540E−11 | −2.740 |
| REACTOME_C_TYPE_LECTIN_RECEPTORS_CLRS | 9.540E−11 | −2.740 |
| REACTOME_DECTIN_1_MEDIATED_NONCANONICAL_NF_{—} | 1.979E−10 | −2.751 |
| KB_SIGNALING | | |
| REACTOME_ORC1_REMOVAL_FROM_CHROMATIN | 4.953E−11 | −2.752 |
| REACTOME_SWITCHING_OF_ORIGINS_TO_A_POST_{—} | 4.953E−11 | −2.752 |
| REPLICATIVE_STATE | | |
| REACTOME_CYCLIN_A_CDK2_ASSOCIATED_EVENTS_AT_{—} | 1.872E−10 | −2.753 |
| S_PHASE_ENTRY | | |
| REACTOME_SCF_SKP2_MEDIATED_DEGRADATION_OF_P27_{—} | 1.872E−10 | −2.753 |
| P21 | | |
| REACTOME_PCP_CE_PATHWAY | 7.319E−11 | −2.765 |
| REACTOME_ASSEMBLY_OF_THE_PRE_REPLICATIVE_{—} | 3.530E−11 | −2.768 |
| COMPLEX | | |
| REACTOME_GLI3_IS_PROCESSED_TO_GLI3R_BY_THE_{—} | 3.131E−11 | −2.774 |
| PROTEASOME | | |
| REACTOME_CDK_MEDIATED_PHOSPHORYLATION_AND_{—} | 3.086E−11 | −2.776 |
| REMOVAL_OF_CDC6 | | |
| REACTOME_G1_S_DNA_DAMAGE_CHECKPOINTS | 3.086E−11 | −2.776 |
| REACTOME_AUF1_HNRNP_D0_BINDS_AND_DESTABILIZES_{—} | 8.077E−11 | −2.777 |
| MRNA | | |
| REACTOME_ACTIVATION_OF_NF_KAPPAB_IN_B_CELLS | 2.142E−11 | −2.778 |
| REACTOME_DOWNSTREAM_SIGNALING_EVENTS_OF_B_{—} | 2.142E−11 | −2.778 |
| CELL_RECEPTOR_BCR | | |
| REACTOME_G2_M_CHECKPOINTS | 8.262E−12 | −2.787 |
| REACTOME_REGULATION_OF_PTEN_STABILITY_AND_{—} | 1.055E−11 | −2.797 |
| ACTIVITY | | |
| REACTOME_REGULATION_OF_RUNX2_EXPRESSION_AND_{—} | 4.635E−11 | −2.797 |
| ACTIVITY | | |
| REACTOME_TRANSCRIPTIONAL_REGULATION_BY_RUNX2 | 4.635E−11 | −2.797 |
| REACTOME_APC_C_CDH1_MEDIATED_DEGRADATION_OF_{—} | 4.873E−11 | −2.809 |
| CDC20_AND_OTHER_APC_C_CDH1_TARGETED_PROTEINS_{—} | | |
| IN_LATE_MITOSIS_EARLY_G1 | | |
| REACTOME_ASYMMETRIC_LOCALIZATION_OF_PCP_{—} | 4.873E−11 | −2.809 |
| PROTEINS | | |
| REACTOME_CROSS_PRESENTATION_OF_SOLUBLE_{—} | 4.873E−11 | −2.809 |
| EXOGENOUS_ANTIGENS_ENDOSOMES | | |
| REACTOME_DEGRADATION_OF_AXIN | 4.873E−11 | −2.809 |
| REACTOME_REGULATION_OF_RUNX3_EXPRESSION_AND_{—} | 4.873E−11 | −2.809 |
| ACTIVITY | | |
| REACTOME_RUNX1_REGULATES_TRANSCRIPTION_OF_{—} | 4.873E−11 | −2.809 |
| GENES_INVOLVED_IN_DIFFERENTIATION_OF_HSCS | | |
| REACTOME_STABILIZATION_OF_P53 | 4.873E−11 | −2.809 |
| REACTOME_TRANSCRIPTIONAL_REGULATION_BY_RUNX3 | 4.873E−11 | −2.809 |
| REACTOME_UBIQUITIN_MEDIATED_DEGRADATION_OF_{—} | 4.873E−11 | −2.809 |
| PHOSPHORYLATED_CDC25A | | |
| REACTOME_KEAP1_NFE2L2_PATHWAY | 8.065E−12 | −2.809 |
| REACTOME_ACTIVATION_OF_APC_C_AND_APC_C_CDC20_{—} | 1.293E−11 | −2.809 |
| MEDIATED_DEGRADATION_OF_MITOTIC_PROTEINS | | |
| REACTOME_DEGRADATION_OF_DVL | 4.542E−12 | −2.823 |
| REACTOME_REGULATION_OF_RAS_BY_GAPS | 4.542E−12 | −2.823 |
| REACTOME_HEDGEHOG_ON_STATE | 4.075E−12 | −2.843 |
| REACTOME_HEDGEHOG_LIGAND_BIOGENESIS | 1.773E−12 | −2.852 |
| REACTOME_INTERLEUKIN_1_FAMILY_SIGNALING | 1.779E−12 | −2.873 |
| REACTOME_TRANSCRIPTIONAL_REGULATION_BY_RUNX1 | 5.166E−12 | −2.878 |

Small RNA sequencing was performed on RBC-EV treatment (PBS, N1, SOD and N1/SOD) groups to identify if there were any molecular changes in the microRNA signature as a response to supplementation. As a percentage of total reads, microRNA content was found to be similar between samples and groups, making up around 5-15% of total mapped reads (FIG. 33A). Further, out of the 260 total microRNA detected, it was identified that in all samples the top 11 microRNA were highly abundant and made up a significant fraction of the total reads, with red blood cell-enriched microRNA miR-451 found to be the most abundant (FIG. 33B). Pathway analysis of known targets of the top 11 microRNA was associated with processes including regulation, transcription, biogenesis, and senescence (FIG. 33C). Venn diagram showed that there were no unique proteins between groups, suggesting that supplementation did not cause any changes to the microRNA signature of RBC-EV (FIG. 33D).

Overall, these results support a unique signature of proteins in RBC and RBC-EV groups, with N1/SOD combination supplementation producing the most proteomic changes in the resultant EV populations compared to other supplementation groups or controls. Further, it was found that RBC incubated in N1/SOD supplements had an anti-inflammatory profile, which was reflected in RBC-EV (N1/SOD).

### Example 26: RBC-EV have Anti-Inflammatory Properties and can Reduce Inflammatory Cytokine Release from Peripheral Bone Mononuclear Cells (PBMC)

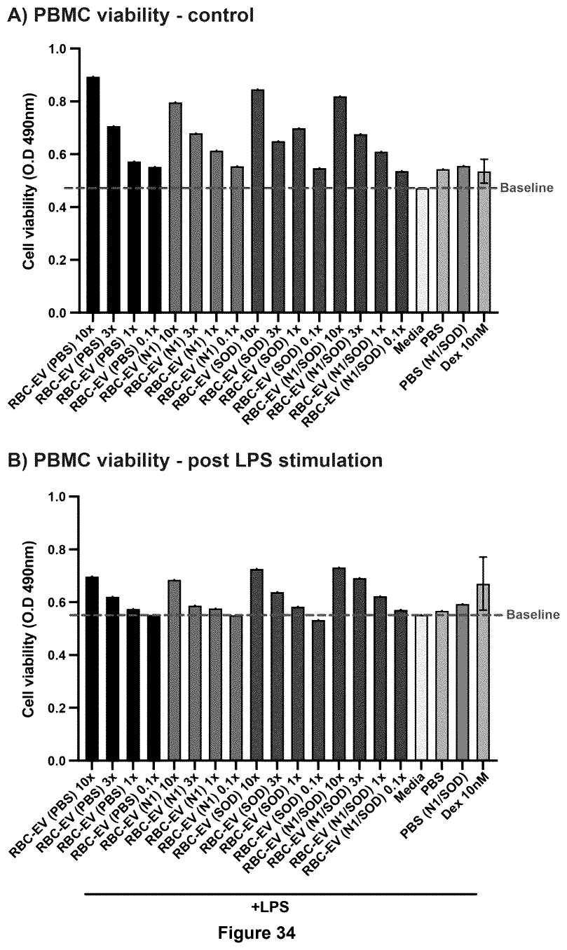

In order to verify the immuno-modulatory properties of RBC-EV identified from multiomic analyses, RBC-EV with and without supplements were sent for independent validation for measures of viability, safety, and anti-inflammatory properties. RBC-EV were incubated on peripheral bone mononuclear cells (PBMC) with and without inflammatory stimulation (LPS). Further, dose responses were also evaluated. Cytokine outputs and viability measures were compared to control groups (media, PBS, PBS (N1/SOD), and dexamethasone 10 nm). Results showed a dose-dependent increase in PBMC viability in response to increased RBC-EV concentration, regardless of supplementation group in both control (FIG. 34A) and LPS-stimulated (FIG. 34B) cells. While no between group difference was noted in the control condition, RBC-EV (N1/SOD) overall promoted higher PBMC viability at each dose compared to RBC-EV (PBS). No change in viability was seen in control groups in either condition, supporting a role for RBC-EV in promoting cell proliferation/viability.

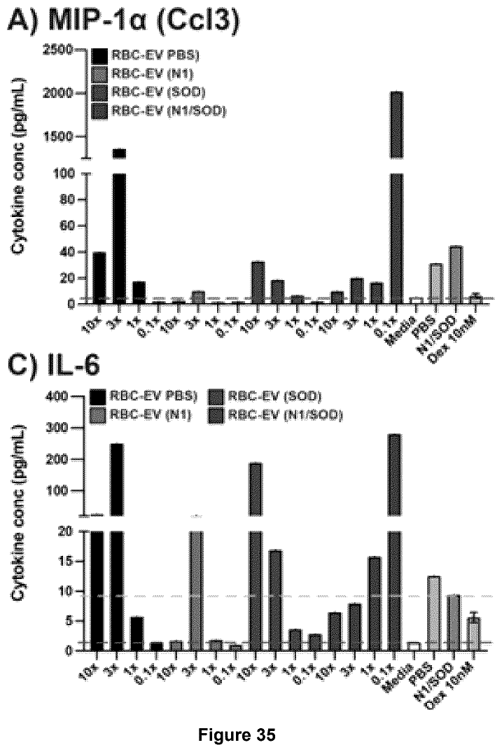

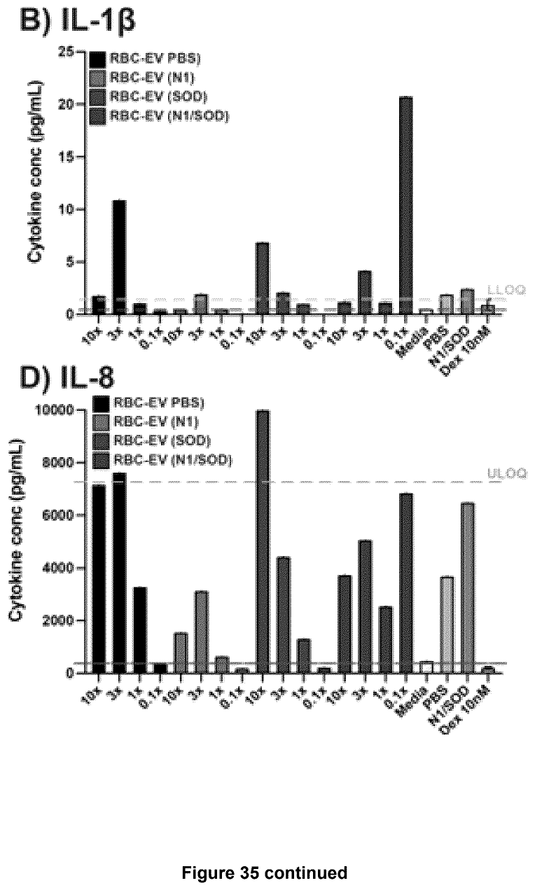

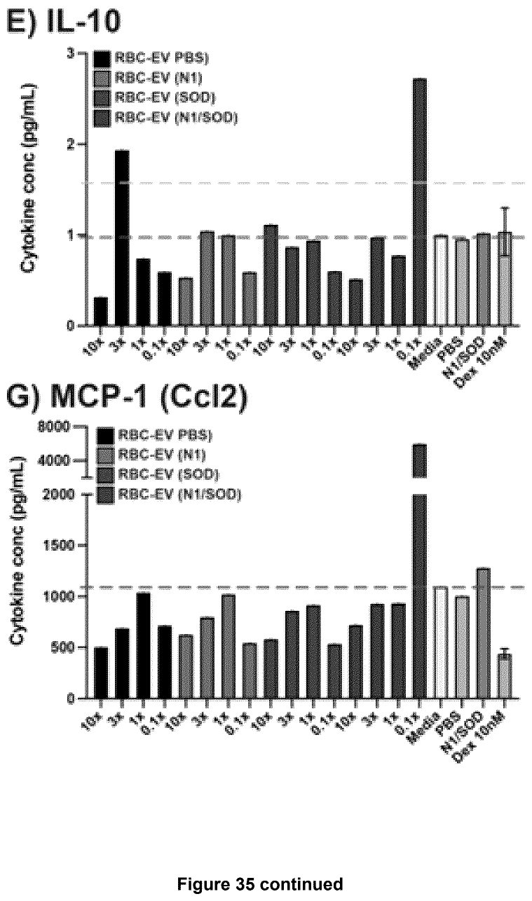

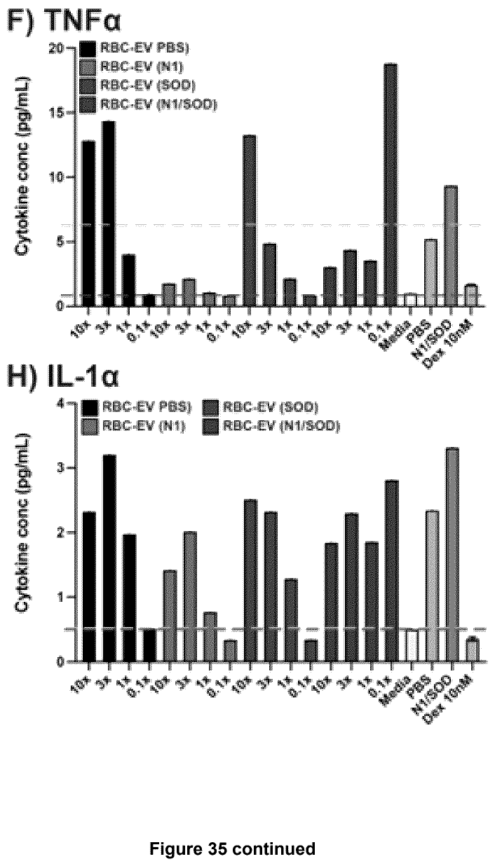

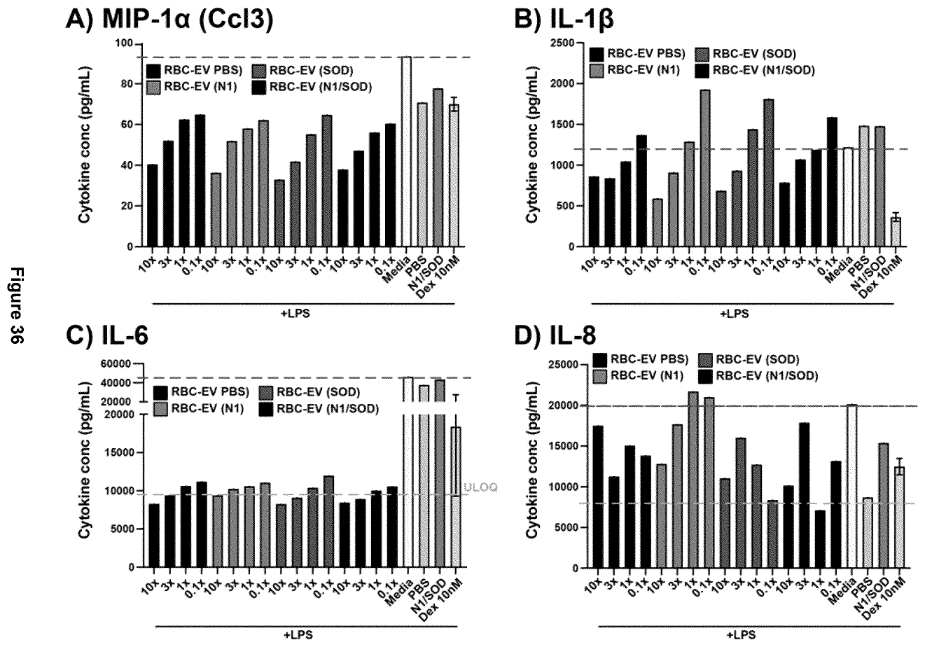

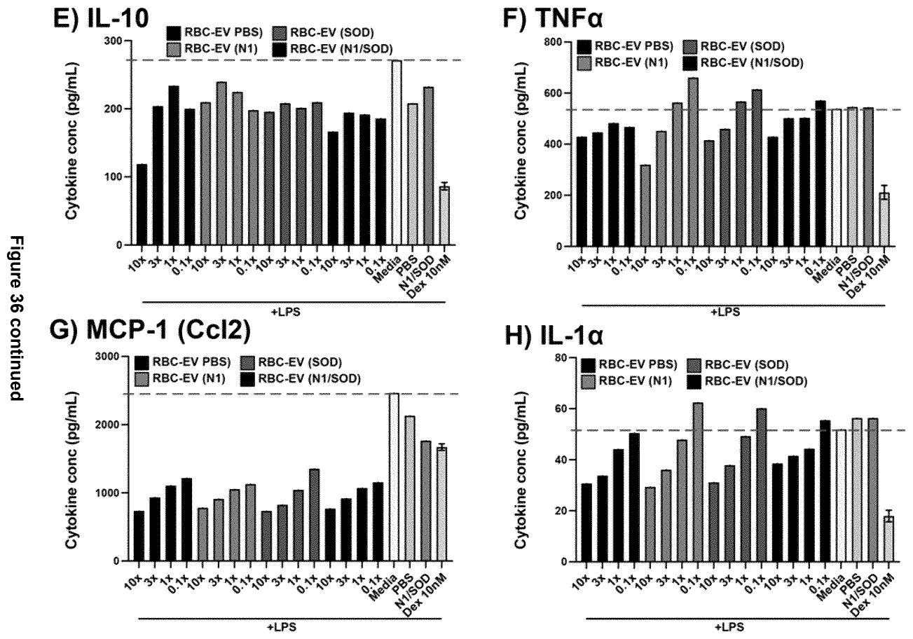

RBC-EV were assessed for their safety profile on PBMCs, with cytokine output (MIP-1α (Ccl3), IL-1β, IL-6, IL-8 IL-10, TnFα, MCP-1 (Ccl2) and IL-1α) measured following 48 hours incubation. These cytokines play known pathogenic roles in both retinal and neurodegenerative diseases (Kauppinen et al., 2016; Wooff et al., 2019; Nagatsu et al., 2005). Overall trends demonstrated a dose-dependent increase in cytokine production for RBC-EV (PBS), RBC-EV (N1), and RBC-EV (SOD), albeit at largely low levels (FIG. 35). Conversely, a dose-dependent decrease in cytokine production was shown in PBMCs treated with RBC-EV (N1/SOD). RBC-EV (N1/SOD) did not induce any robust cytokine release above baseline levels (comparative to media control, and above LLOQ) with the exception of IL-8 and IL-1α. Overall these results support the safety of RBC-EV (N1/SOD) and suggest a strong dose-dependent response to reduce cytokine production.

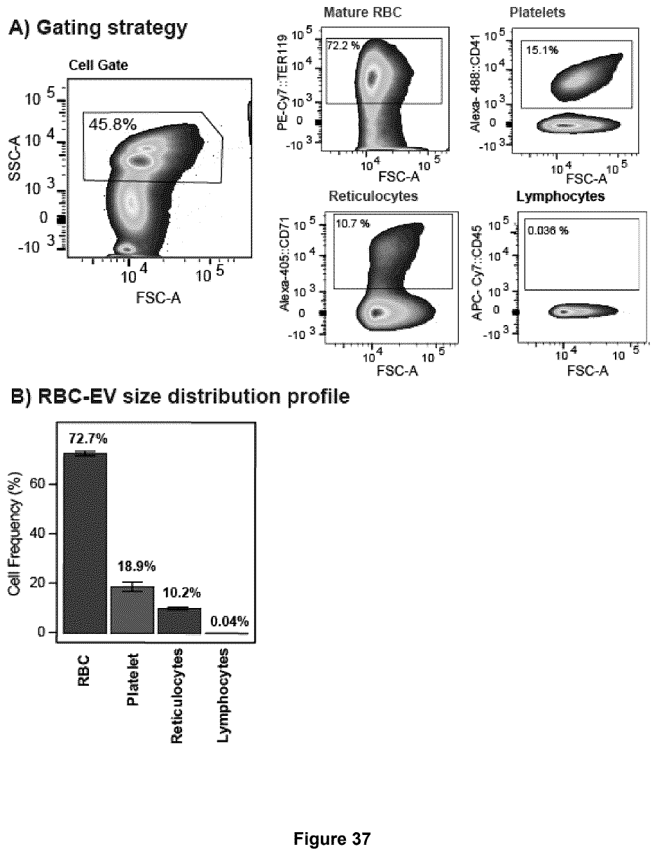

Finally, RBC-EV were assessed for their anti-inflammatory properties using the same cytokine outputs and following 48 hours LPS stimulation and EV incubation. Results showed a clear anti-inflammatory response with a dose-dependent decrease in all cytokine production in all RBC-EV groups (FIG. 36). Importantly, RBC-EV (N1/SOD) was able to reduce cytokine output below levels of anti-inflammatory steroid dexamethasone (10 nM) for MIP-1α (Ccl3), IL-6, IL-8 and MCP-1 (Ccl2) (FIGS. 36A, C, D and G). Taken together, these results support a strong anti-inflammatory mechanism of action for RBC-EV and the ability to reduce key cytokines known to be involved in both retinal and neurodegenerative diseases.

### Example 27: Measuring RBC, Platelets, Leukocytes, and Reticulocytes in RBC-Enriched Fraction of Whole Mouse Blood

Flow cytometry was used to determine the proportion of red blood cells (RBCs) after plasma removal and leukocyte depletion. Cells were labelled using fluorescently conjugated antibodies specific to various cell lineages: TER119 for erythroid cells, CD41 for megakaryocytes, CD71 for reticulocytes, and CD45 for lymphoid cells. A gating strategy was applied to isolate populations of TER119+, CD41+, CD71+, and CD45+ cells, as shown in FIG. 37A. This analysis demonstrated that mature RBCs and reticulocytes together comprised approximately 83% of the total cell population. Additionally, CD41+ platelets represented 15-19% and leukocytes were less than 0.04% of the cells, as illustrated in FIG. 37B. This suggests that EV population derived post-incubation would consist of both RBC and platelet-derived vesicles.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

This application claims priority from Australian Provisional Application No. 2023901479 entitled “Methods of producing extracellular vesicles and uses thereof” filed on 15 May 2023, and Australian Provisional Application No. 2023901480 entitled “Extracellular vesicles and uses thereof” filed on 15 May 2023, the entire contents of which are hereby incorporated by reference.

All publications discussed and/or referenced herein are incorporated herein in their entirety.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

### REFERENCES

Al-Ubaidi et al (1992) Proceedings of the National Academy of Sciences of the United States of America 89(4):1194-1198
Al-Ubaidi et al (2013) Investigative Ophthalmology & Visual Science 54(13):8119-24
Amin et al (2020) Translation Psychiatry 10:267.
Araújo et al (2022) Cells 11(18):2908.
Averill-Bates (2023) Vitam Horm 121:109-41.
Benatar et al (2022) Brain 145(1):27-44.
Bjelobaba et al (2017) Current Pharmaceutical Design 23(5):693-730.
Blasi et al (1990) Journal of Neuroimmunology 27(2-3):229-37.
Cherry et al (2022) Journal of Neuroinflammation 19:278.
Chiangjong et al (2021) Frontiers in Medicine doi: 10.3389/fmed.2021.761362
Deacon (2013) J Vis Exp (75):e2609.
Dellar et al (2022) Journal of Extracellular Biology 1:e40.
Dunn et al (1996) Experimental Eye Rsearch 62(2):155-169.
Fernando et al (2018) Investigative Ophthalmology & Visual Science 59(11):4362-74.
Gangadaran et al (2018) Frontiers in Pharmacology doi: 10.3389/fphar.2018.00817
Jacob et al (1991). Cytometry, 12(6), 550-558. doi: 10.1002/cyto.990120612
Jayaraj et al (2019) Journal of Neuroinflammation 16:142.
Josefsberg et al. (2012) Biotech and Bioengineering. 97(1):73-85.
Kauppinen et al (2016) Cellular and molecular life sciences 73(9):1765-86.
Koster et al (2022) International Journal of Molecular Sciences 23(6).
Lamichhane and Jay (2018) Methods Molecular Biology 1831:37-47.
Laurent et al (2018) Biomedical Journal 41(1):21-33.
Lee et al (2021) doi: 10.1007/978-1-0716-1712-0_9
Leng et al (2021) Nature Reviews Neurology 17:157-172.
Lieto et al (2022) International Journal of Molecular Science 23(16):9158.
Limb et al (2002) Investigative Ophthalmology & Visual Science 43(3):864-869.
Lu et al (2017) Experimental Eye Research 165:78-89.
Masini et al (2021) Biomedicines 9(6):598.
Mead et al (2023) Nature Reviews Drug Discovery 22:185-212.
Naegele et al (2014) Handbook Clinical Neurology 122:59-87
Nagatsu and Sawada (2005) Curr Pharm Des 11(8):999-1016.
Natoli et al (2010) Molecular Vision 16:1801-22.
Natoli et al (2016) Experimental Eye Research 147:114-127.
Nederveen et al (2021) Frontiers in Physiology 11:2020.
Palpagama et al (2019) Frontiers in Molecular Neuroscience 12:258.
Rojas et al (2020) doi: 10.3389/fnins.2020.566858
Rutar et al (2015) Journal of Neuroinflammation 12(1):8.
Salehi et al (2018) Biomedicine 6(3).
Sánchez-Sarasúa et al (2020) International Journal of Molecular Science 21(22):8751.
Silva (2012) Genes Chromosom Cancer 51.
Sreekumar et al (2021) Antioxidants 10(5).
Jacob et al (1991) Cytometry, 12(6), 550-558.
Usman et al (2018) Nature Communications 9(1):2359.
Wolf et al (2011) Expert Rev Vaccine. 10(10):1451-1475.
Wooff et al (2019) Frontiers in Immunology 10:1618.
Wooff et al (2020) Frontiers in Neuroscience 14.160.

1. A method of producing extracellular vesicles (EVs) comprising incubating or culturing EV producing cells in media comprising an antioxidant and/or an N1 media component or an equivalent thereof.

2. The method of claim 1, wherein the antioxidant comprises one or both of an enzymatic antioxidant and a non-enzymatic antioxidant.

3. The method of claim 2, wherein the enzymatic antioxidant is selected from one or more of: superoxide dismutase (SOD), manganese superoxide dismutase (SOD2), catalase, peroxiredoxin, glutathione peroxidase, and glutathione reductase.

4. The method of any one of claims 1 to 3, wherein the enzymatic antioxidant is superoxide dismutase (SOD).

5. The method of any one of claims 1 to 4, wherein the non-enzymatic antioxidant is selected from one or more of: haemoglobulin, kaempferol, glutathione, vitamin E, vitamin A, vitamin C, tocopherol, carotenoid, glutathione and curcumin.

6. The method of any one of claims 1 to 5, wherein the N1 media component is selected from one or more of: transferrin, insulin, sodium selenite, putrescine and progesterone or an equivalent thereof.

7. The method of claim 6, wherein the N1 media component is transferrin or an equivalent thereof.

8. The method of any one of claims 1 to 7, wherein the media comprises N1 media.

9. The method of any one of claims 1 to 8, wherein the media comprises N1 media and SOD.

10. The method of any one of claims 1 to 9, wherein the EV producing cells are human cells.

11. The method of any one of claims 1 to 10, wherein the EV producing cells are selected from: red blood cells, reticulocytes, mesenchymal stem cells, epithelial cells, endothelial progenitor cells, umbilical cord cells, an ocular cell line, a neuronal cell line, dental pulp cells, dendritic cells, white blood cells, cancer cells, microglial cells, glial cells, astrocytes, photoreceptor cells and embryonic fibroblasts.

12. The method of claim 11, wherein the cells are red blood cells.

13. The method of claim 11 or 12, wherein the cells are red blood cells and reticulocytes.

14. The method of claim any one of claims 1 to 13, wherein the EV producing cells are present with EV producing cell fragments.

15. The method of claim 14, wherein the EV producing cell fragments are platelets.

16. The method of any one of claims 12 to 15, wherein the red blood cells are diluted about 1:10 before addition to the media.

17. The method of claim 11, wherein the ocular cell line is a retinal cell line selected from: aRPE19, BV2, MIO-M1, D407 and iMG.

18. The method of any one of claims 1 to 17, wherein the EVs are isolated after incubation/culturing via centrifugation.

19. The method of any one of claims 1 to 18, wherein when the method comprises incubating, the media comprises phosphate buffered saline (PBS).

20. The method of any one of claims 1 to 19, wherein when the method comprises incubating or culturing for about 10 hours to about 24 hours, or about 12 to about 20 hours, or about 12 hours to about 18 hours, or about 18 hours.

21. The method of claim 20, wherein the method comprises incubating for about 16 hours.

22. The method of any one of claims 1 to 21, wherein the method increases the quality and/or quantity and/or efficacy of the EVs produced.

23. The method of claim 22, wherein increasing quality comprises increasing the uniformity of EV vesicle size.

24. The method of claim 23, wherein increasing quality comprises increasing the level of one or both of an endogenous and an exogenous antioxidant in the EVs.

25. The method of any one of claims 1 to 24, wherein the method increases the level of one or both of an endogenous and an exogenous antioxidant in the EV producing cells.

26. The method of any one of claims 1 to 25, wherein the method reduces the percent of EV producing cells with abnormal morphology compared to EV producing cells incubated or cultured in media not comprising the antioxidant and/or an N1 media component or an equivalent thereof.

27. The method of claim 26, wherein less than about 10% of the EV producing cells have abnormal morphology.

28. The method of claim 24 or claim 25, wherein the endogenous antioxidant is selected from one or more of: haemoglobin, SOD, glutathione, vitamin C, vitamin E, catalase and glutathione peroxidase.

29. The method of claim 24 or claim 25, wherein the exogenous antioxidant is selected from one or more of: SOD, and kaempferol.

30. A population of extracellular vesicles (EVs) derived from cells comprising an increased level one or both of an endogenous and an exogenous antioxidant.

31. A population of extracellular vesicles (EVs) produced by the method of any one of claims 1 to 29.

32. The population of EVs of claim 30 or 31, wherein the EVs comprise increased expression of one or more proteins compared to a control, wherein the one or more proteins are selected from: ARIH1, RPL22, CFI, LNPK, RPS17, RPL23A, RPS21, CPOX, METAP2, FARSB, RPL18, MYG1, RPS3, RPS15A, HSP90B1, PHB1, EEF1D, DDX1, RPS4X, EIF2S3Y, AHSG, RPL12, NDUFA4, SACM1L, RPS5, RPL18A, RPL14, CALR, RPL26, RPLP0, HYOU1, ENO3, CTSE, ALDH1A7, PDIA6, PRKCSH, CLNS1A, RPS6, HACE1, RPL17, RPL27A, EPRS1, PRXL2A, TRIM56, CANX, NPEPL1, PDIA3, PPIB, EEF2, SPR, NGP, HSPA5, BAG2, SND1, RANGAP1, ENO1, RPS14, TFRC, THG1L, PFAS, PPP2R1A, GLO1 and SCAMP3.

33. The population of EVs of any one of claims 30 to 32, wherein the EVs comprise decreased expression of one or more proteins compared to a control, wherein the one or more proteins are selected from: FCHO2, CRLF3, PSMA5, GSPT1, PSMB4, ACP1, CCDC6, UROS, PSMD3, TGM2, PSMC2, PSMA3, GLRX3, RNH1, PPID, USP25, GMPR, USP5, PGLS, OSTF1, PSMB6, EIF5, TBCB, OXSR1, STIP1, USP14, PSMD5, TOLLIP, PSMD8, GPI, OTUB1, SRI, AGFG1, CFAP157, PSMD6, CHORDC1, PHPT1, PSMD7, PSMD11, PSMD12, PSMD13, GSTM5, SYNJ1, SH3GLB1, SWAP70, H4C1, PZP, PSMD14, WNK1, GDI1, PF4, SNX15, CORO1C, PTGR2, ALDOART2, IGHG3, LZIC, EPN1, PACS1, SKIC2, KYAT3, RNF213A and ANXA6.

34. The population of EVs of any one of claims 30 to 33, wherein the EVs comprise increased expression of one or more proteins compared to a control, wherein the one or more proteins mediate one or more pathway/s selected from: Rab regulation of trafficking, Srp dependent cotranslational protein targeting to membrane, major pathways of rRNA processing in the nucleolus and cytosol, nonsense mediated decay independent of the exon junction complex, nonsense mediated decay nmd, raf gefs exchange gtp for gdp on rabs, cytoplasmic ribosomal proteins, trans golgi network vesicle budding, formation of a pool of free 40s subunits, Golgi associated vesicle biogenesis, MHC class II antigen presentation, Rab geranylgeranylation, signaling by ntrk1 trka, signaling by ntrks, vesicle mediated transport, eukaryotic translation initiation, signaling by receptor tyroskine kinases, neurotransmitter receptors and postsynaptic signal transmission, membrane trafficking and clathrin mediated endocytosis

35. The population of EVs of any one of claims 30 to 34, wherein the EVs comprise decreased expression of one or more proteins compared to a control, wherein the one or more proteins mediate one or more pathways selected from: Interleukin 1 family signaling, transcriptional regulation by runx1, Hedgehog ligand biogenesis, Hedgehog on state, ubiquitin mediated degradation of cdc25a, transcriptional regulation by runx3, stabilization of p53, runx1 regulates transcription of genes in differentiation of hscs, regulation of runx3 expression and activity, degradation of axin, cross presentation of soluble exogenous antigens endosomes, asymmetric localization of pcp proteins, Apc c cdh1 mediated degradation of cdc20 targeted proteins in late mitosis G1, regulation of ras by gaps, degradation of dv1, keap1 nef212 pathway, activation of apc c and apc c cdc20 mediated degradation of mitotic proteins, pcp ce pathway, transcriptional regulation by runx2, regulation of runx2 expression and activity, regulation of pten stability and activity, gli3 is processed to gli3r by the proteasome, G2 m checkpoints, auf1 hnrnp do binds and destabilizes mRNA, switching of origins to a post replicative state, Orc1 removal from chromatin, apc c mediated degradation of cell cycle proteins, Tnfr2 non canonical nf kb pathway, Ub specific processing proteases and beta catenin independent wnt signaling.

36. The population of EVs of any one of claims 30 to 35, wherein the endogenous antioxidant is selected from one or more of: haemoglobin, SOD, glutathione, vitamin C, vitamin E, catalase and glutathione peroxidase.

37. The population of EVs of any one of claims 30 to 36, wherein the exogenous antioxidant is selected from one or more of: SOD and kaempferol.

38. The population of EVs of any one of claims 30 to 37, wherein the EVs have a mean diameter of less than 200 nm.

39. The population of EVs of any one of claims 30 to 38, wherein when administered systemically or locally a portion of the EVs localise to the eye.

40. The population of EVs of any one of claims 30 to 39, wherein when administered systemically or locally a portion of the EVs localise to the retina.

41. The population of EVs of any one of claims 30 to 40, wherein when administered systemically or locally a portion of the EVs localise to the brain.

42. The population of EVs of any one of claims 30 to 41, wherein when administered systemically or locally a portion of the EVs localise to one or more of: microglial cells, glial cells, and neurons.

43. The population of EVs of any one of claims 30 to 42, wherein the EVs further comprise an exogenous cargo.

44. The population of EVs of claim 43, wherein the exogenous cargo is selected from one or more of: a drug, antioxidant, chemotherapy, protein, lipid, nucleic acid (such as DNA, mRNA, miRNAs, siRNAs, circular RNA, long non-coding RNA and snoRNAs), CRISPR/Cas9, nanoparticle and an exogenous targeting molecule.

45. The population of EVs of any one of claims 30 to 44, wherein the EVs are autologous.

46. The population of EVs of any one of claims 30 to 45, wherein the EVs comprise an endogenous cargo, wherein the endogenous cargo is an miRNA selected from one or more of: mmu-miR-142a-3p, mmu-miR-486b-3p, mmu-let-7c-5p, mmu-miR-16-5p, mmu-miR-25-3p, mmu-miR-486a-3p, mmu-miR-486b-5p, mmu-miR-486a-5p, mmu-let-7f-5p, mmu-let-7a-5p and mmu-miR-451a.

47. A composition comprising EVs produced by the method of any one of claims 1 to 29, or a population of EVs of any one of claims 30 to 46 for use in treating and/or preventing a disease or condition in a subject.

48. A composition comprising EVs produced by the method of any one of claims 1 to 29, or a population of EVs of any one of claims 30 to 46 for use in treating and/or preventing neurodegeneration in a subject.

49. A method of treating and/or preventing neurodegeneration in a subject, the method comprising administering EVs produced by the method of any one of claims 1 to 29, or a population of EVs of any one of claims 30 to 46 to a subject.

50. Use of EVs produced by the method of any one of claims 1 to 29, or a population of EVs of any one of claims 30 to 46 for treating and/or preventing neurodegeneration in a subject.

51. Use of EVs produced by the method of any one of claims 1 to 29, or a population of EVs of any one of claims 30 to 46 in the manufacture of a medicament for treating and/or preventing neurodegeneration in a subject.

52. A composition comprising EVs produced by the method of any one of claims 1 to 29, or a population of EVs of any one of claims 30 to 46 for use in reducing oxidative stress and/or inflammation in the eye of a subject.

53. A method of reducing oxidative stress and/or inflammation in the eye of a subject, the method comprising administering EVs produced by the method of any one of claims 1 to 29, or a population of EVs of any one of claims 30 to 46 to a subject.

54. Use of EVs produced by the method of any one of claims 1 to 29, or a population of EVs of any one of claims 30 to 46 for reducing oxidative stress and/or inflammation in the eye of a subject.

55. Use of EVs produced by the method of any one of claims 1 to 29, or a population of EVs of any one of claims 30 to 46 in the manufacture of a medicament for reducing oxidative stress and/or inflammation in the eye of a subject.

56. A composition comprising EVs produced by the method of any one of claims 1 to 29, or a population of EVs of any one of claims 30 to 46 for use in treating and/or preventing retinal degeneration in a subject.

57. A method of treating and/or preventing retinal degeneration in a subject, the method comprising administering EVs produced by the method of any one of claims 1 to 29, or a population of EVs of any one of claims 30 to 46 to a subject.

58. Use of EVs produced by the method of any one of claims 1 to 29, or a population of EVs of any one of claims 30 to 46 for treating and/or preventing retinal degeneration in a subject.

59. Use of EVs produced by the method of any one of claims 1 to 29, or a population of EVs of any one of claims 30 to 46 in the manufacture of a medicament for treating and/or preventing retinal degeneration in a subject.

60. The composition, use or method of any one of claims 47 to 59, wherein the EVs are or will be administered systemically.

61. The composition, use or method of claim 60, wherein systemic administration comprises intravenous administration or infusion.

62. The composition, use or method of any one of claims 47 to 59, wherein the EVs are or will be administered locally.

63. The composition, use or method of claim 62, wherein local administration is selected from: topical administration to the eye, intraocular administration, sub retinal administration, intravitreal administration, intracranial injection, intrathecal injection, intracerebral infusion and intracerebral implantation.

64. The composition, use or method of any one of claims 47 to 63, wherein the neurodegeneration comprises neuroinflammation.

65. The composition, use or method of any one of claims 47 to 64, wherein the neurodegeneration is selected from: Parkinson's disease, Amyotrophic Lateral Sclerosis, Alzheimer's disease, a tauopathy, Multiple Sclerosis, Lewy Body Dementia, stroke, transient ischemic attack, and Huntington's disease.

66. The composition, use or method of any one of claims 47 to 65, wherein the neurodegeneration is Parkinson's disease.

67. The composition, use or method of any one of claims 47 to 66, wherein the neurodegeneration is Alzheimer's disease.

68. The composition, use or method of any one of claims 48 to 51, wherein the retinal degeneration is selected from: macular degeneration, retinitis pigmentosa and diabetic retinopathy.

69. The composition, use or method of claim 68, wherein the macular degeneration is selected from wet macular degeneration and dry macular degeneration.

70. The composition, use or method of any one of claims 52 to 55, wherein the oxidative stress and/or inflammation is caused by and/or is the result of a disease or condition selected from: macular degeneration, retinitis pigmentosa, diabetic retinopathy, stargardt disease, leber congenital amaurosis, cone-rod dystrophy, usher syndrome, choroideremia, bardet-biedl syndrome, macular telangiectasia, macular oedema, retinal detachment, retinal ischemia, uveitis, scleritis, conjunctivitis, keratitis, corneal ulcer, glaucoma trachoma, choroidal melanoma, ocular melanoma, glaucoma retinal dystrophy, strabismus and cataracts.

71. A method of producing extracellular vesicles (EVs) comprising incubating or culturing EV producing cells and fragments of EV producing cells in media comprising an antioxidant and/or an N1 media component or an equivalent thereof.

72. The method of claim 71, wherein the fragments of EV producing cells are platelets.
